(19) United States
(12) Patent Application Publication
(10) Pub. No.: US 2024/0197641 A1
(43) Pub. Date: Jun. 20, 2024

(54) COMPOSITIONS AND METHODS FOR TARGETED SYSTEMIC DELIVERY TO CELLS

(71) Applicants: ReCode Therapeutics, Inc., Menlo Park, CA (US); The Board of Regents of the University of Texas System, Austin, TX (US)

(72) Inventors: David J. Lockhart, Menlo Park, CA (US); Vladimir Kharitonov, San Diego, CA (US); Brandon Wustman, San Diego, CA (US); Daniel Siegwart, Austin, TX (US); Xueliang Yu, Menlo Park, CA (US); Jackson Eby, Menlo Park, CA (US)

(21) Appl. No.: 18/283,615

(22) PCT Filed: Mar. 23, 2022
(86) PCT No.: PCT/US2022/021553

§ 371 Date: Sep. 22, 2023

Related U.S. Application Data
(60) Provisional application No. 63/164,534, filed on Mar. 23, 2021.
(60) Provisional application No. 63/305,652, filed on Feb. 1, 2022.

(51) Int. Cl. A61K 9/51; A61K 9/00; A61K 31/7105

(52) U.S. Cl. CPC A61K 9/5123; A61K 9/0019; A61K 31/7105

(57) ABSTRACT

Described herein are compositions, kits, and methods for potent systemic delivery to a cell of a subject. Also described herein are pharmaceutical compositions comprising a therapeutic or prophylactic agent assembled to a lipid composition. The lipid composition can comprise an ionizable cationic lipid, a phospholipid, and a selective organ targeting lipid. Further described herein are high-potency dosage forms of a therapeutic or prophylactic agent formulated with a lipid composition.

### CROSS-REFERENCE

This application claims the benefits of U.S. Provisional Application No. 63/164,534 filed on Mar. 23, 2021, and U.S. Provisional Application No. 63/305,652 filed on Feb. 1, 2022, the entirety of which are incorporated herein by reference for all purposes.

### BACKGROUND

Therapeutic approaches, such as the CRISPR/Cas (clustered regularly interspaced short palindromic repeat/CRISPR-associated protein (Cas)) technology, often require precise and potent delivery of therapeutic agent(s) to target organ(s) or cell(s), sometimes in a sequence dependent manner. To date, there remains a clear need to accomplish therapeutically safe and effective lipid-based carriers for achieving clinical outcomes in the context of genetic diseases and many other applications.

### SUMMARY

In certain aspects, the present application provides compositions for potent delivery of a therapeutic agent to a cell of a subject. In some embodiments, the composition can be formulated for systemic (e.g., intravenous) administration, the composition comprising a therapeutic agent assembled with a lipid composition that comprises:
(i) an ionizable cationic lipid;
(ii) a polymer-conjugated lipid; and
iii) a selective organ targeting (SORT) lipid has the structure of Formula (IA), or a pharmaceutically acceptable salt, stereoisomer, tautomer thereof:

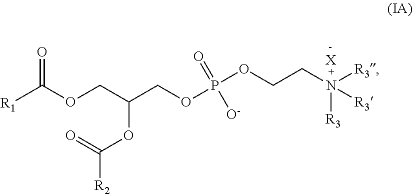

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In some embodiments, the composition can be formulated for systemic (e.g., intravenous) administration, the composition comprising a therapeutic agent assembled with a lipid composition that comprises:
(i) an ionizable cationic lipid;
(ii) a polymer-conjugated lipid; and
iii) a selective organ targeting (SORT) lipid has the structure of Formula (IA), or a pharmaceutically acceptable salt, stereoisomer, tautomer thereof:

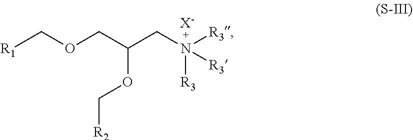

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In certain aspects, the present application provides methods for potent delivery of a therapeutic agent to a cell of a subject. In some embodiments, the methods described herein can be for targeted delivery of a therapeutic agent to a spleen cell, the method comprising (e.g., systemically) administering a composition described herein, thereby providing an effective amount or activity of said therapeutic agent in said spleen cell of said subject that is at least 1.1-fold greater than a corresponding amount or activity of said therapeutic agent achieved in a lung cell of said subject.

In some embodiments, the methods described herein can be for targeted delivery of a therapeutic agent to a lung cell, the method comprising (e.g., systemically) administering a composition described herein, thereby providing an effective amount or activity of said therapeutic agent in said lung cell of said subject that is at least 1.1-fold greater than a corresponding amount or activity of said therapeutic agent achieved in a spleen cell of said subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. The patent or application file contains at least one drawing executed in color. Copies of this patent or patent application publication with color drawing(s) will be provided by the Office upon request and payment of the necessary fee. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows examples of structures of SORT lipids.

FIG. 2 shows example structures of dendrimers or dendrons of the ionizable cationic lipids.

FIG. 3 shows the stability and general characteristics of various LNP compositions.

FIG. 4A shows IVIS organ imaging of spleen, liver and lung of a female dog after the administration of a Lung-SORT.

FIG. 4B shows IVIS organ imaging of spleen, liver and lung of a male dog after the administration of a Lung-SORT.

FIG. 5A shows IVIS organ imaging of spleen, liver and lung of a Cynomolgus NHP after the administration of a Lung-SORT.

FIG. 5B shows IVIS organ imaging of spleen, liver and lung of a Cynomolgus NHP after the administration of a Lung-SORT.

FIG. 6A shows IVIS organ imaging of spleen, liver, kidneys and lung of mice after the administration of luciferase mRNA formulated with 14:0 TAP SORT after 5 hrs.

FIG. 6B quantitatively displays the signal obtained at the lungs, spleen, and liver of the mice in FIG. 6A.

FIG. 7A shows the TR intensity expressed in the treated hBEs of the two different SORT LNPs.

FIG. 7B shows the % LDH released from the treated hBEs of the two different SORT LNPs.

FIG. 8A shows IVIS organ imaging of spleen, liver, and lung of two beagles after the IV bolus administration of luciferase mRNA formulated in 14:0 TAP SORT.

FIG. 8B shows IVIS organ imaging of spleen, liver, and lung of two beagles after the IV infusion of luciferase mRNA formulated in 14:0 TAP SORT with pre-meds.

FIG. 9 shows a compiled panel of IVIS organ imaging of spleen, liver, and lung of dogs and NHPs.

FIG. 10A shows ex vivo imaging of bioluminescence of spleen, lungs, liver and kidneys after IV delivery of a Luc mRNA/LNP using multiple compositions of LNP.

FIG. 10B shows quantititavively graphs the biolumcence data of FIG. 10A.

FIG. 11A shows ex vivo imaging of bioluminescence of spleen, lungs, liver and kidneys after IV delivery of a Luc mRNA/LNP using multiple compositions of LNP.

FIG. 11B shows quantititavively graphs the biolumcence data of FIG. 11A.

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

In the context of the present application, the following terms have the meanings ascribed to them unless specified otherwise:

As used throughout the specification and claims, the terms “a”, “an” and “the” are generally used in the sense that they mean “at least one”, “at least a first”, “one or more” or “a plurality” of the referenced components or steps, except in instances wherein an upper limit is thereafter specifically stated. For example, a “cleavage sequence”, as used herein, means “at least a first cleavage sequence” but includes a plurality of cleavage sequences. The operable limits and parameters of combinations, as with the amounts of any single agent, will be known to those of ordinary skill in the art in light of the present application.

The terms “polypeptide”, “peptide”, and “protein” are used interchangeably herein to generally refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein in the context of the structure of a polypeptide, “N-terminus” (or “amino terminus”) and “C-terminus” (or “carboxyl terminus”) generally refer to the extreme amino and carboxyl ends of the polypeptide, respectively.

The term “N-terminal end sequence,” as used herein with respect to a polypeptide or polynucleotide sequence of interest, generally means that no other amino acid or nucleotide residues precede the N-terminal end sequence in the polypeptide or polynucleotide sequence of interest at the N-terminal end. The term “C-terminal end sequence,” as used herein with respect to a polypeptide or polynucleotide sequence of interest, generally means that no other amino acid or nucleotide residues follows the C-terminal end sequence in the polypeptide or polynucleotide sequence of interest at the C-terminal end.

The terms “non-naturally occurring” and “non-natural” are used interchangeably herein. The term “non-naturally occurring” or “non-natural,” as used herein with respect to a therapeutic agent or prophylactic agent, generally means that the agent is not biologically derived in mammals (including but not limited to human). The term “non-naturally occurring” or “non-natural,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

As used herein, the terms “treatment” or “treating,” or “palliating” or “ameliorating” are used interchangeably herein. These terms generally refer to an approach for obtaining beneficial or desired results including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

A “therapeutic effect” or “therapeutic benefit,” as used herein, generally refers to a physiologic effect, including but not limited to the mitigation, amelioration, or prevention of disease or an improvement in one or more clinical parameters associated with the underlying disorder in humans or other animals, or to otherwise enhance physical or mental wellbeing of humans or animals, resulting from administration of a polypeptide of the disclosure other than the ability to induce the production of an antibody against an antigenic epitope possessed by the biologically active protein. For prophylactic benefit, the compositions may be administered to a subject at risk of developing a particular disease, a recurrence of a former disease, condition or symptom of the disease, or to a subject reporting one or more of the physiological symptoms of a disease, even though a diagnosis of this disease may not have been made.

The terms “therapeutically effective amount” and “therapeutically effective dose”, as used herein, generally refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “equivalent molar dose” generally means that the amounts of materials administered to a subject have an equivalent amount of moles, based on the molecular weight of the material used in the dose.

The term “therapeutically effective and non-toxic dose,” as used herein, generally refers to a tolerable dose of the compositions as defined herein that is high enough to cause depletion of tumor or cancer cells, tumor elimination, tumor shrinkage or stabilization of disease without or essentially without major toxic effects in the subject. Such therapeutically effective and non-toxic doses may be determined by dose escalation studies described in the art and should be below the dose inducing severe adverse side effects.

The terms “cancer” and “cancerous” refer to or describe the physiological condition in mammals that is typically characterized by unregulated cell growth/proliferation.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof, “mercapto” means —SH; and “thio” means ═S; “sulfonyl” means —S(O)₂—; “hydroxysulfonyl” means —S(O)₂OH; “sulfonamide” means —S(O)₂NH₂; and “sulfinyl” means —S(O)—.

In the context of chemical formulas, the symbol “ [symbol] ” means a single bond, “ [symbol] ” means a double bond, and “≡” means triple bond. The symbol “ [symbol] ” represents an optional bond, which if present is either single or double. The symbol “ [symbol] ” represents a single bond or a double bond. Thus, for example, the formula

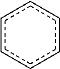

includes

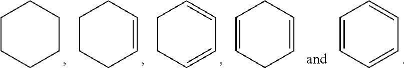

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

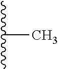

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

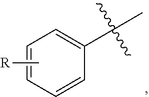

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

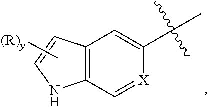

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group atom means the compound or chemical group contains a planar unsaturated ring of atoms that is stabilized by an interaction of the bonds forming the ring.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ⁱBu), and —CH₂C(CH₃)₃(neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. An “alkane” refers to the class of compounds having the formula H—R, wherein R is alkyl as this term is defined above.

When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, the carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

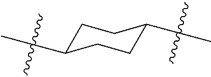 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to an monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, the carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, the carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

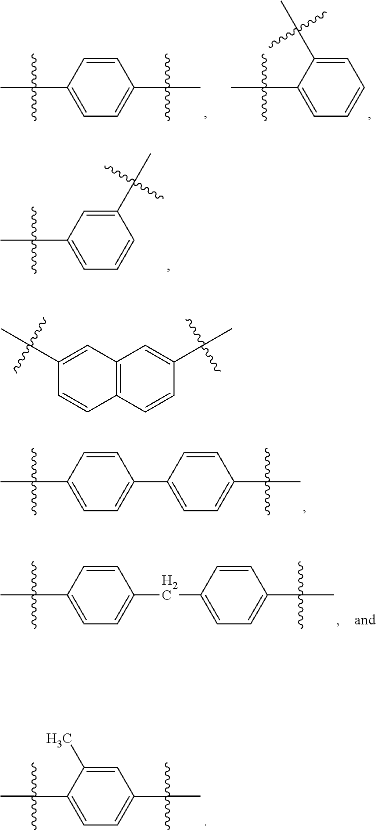

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, the carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. Heteroaryl rings may contain 1, 2, 3, or 4 ring atoms selected from are nitrogen, oxygen, and sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. The term “heteroarenediyl” when used without the “substituted” modifier refers to an divalent aromatic group, with two aromatic carbon atoms, two aromatic nitrogen atoms, or one aromatic carbon atom and one aromatic nitrogen atom as the two points of attachment, the atoms forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl, aryl, and/or aralkyl groups (carbon number limitation permitting) attached to the aromatic ring or aromatic ring system. Non-limiting examples of heteroarenediyl groups include:

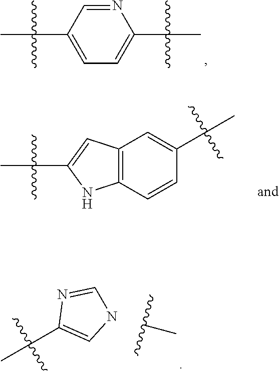

A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “heterocycloalkyl” when used without the “substituted” modifier refers to a monovalent non-aromatic group with a carbon atom or nitrogen atom as the point of attachment, the carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. Heterocycloalkyl rings may contain 1, 2, 3, or 4 ring atoms selected from nitrogen, oxygen, or sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” when used without the “substituted” modifier refers to a divalent cyclic group, with two carbon atoms, two nitrogen atoms, or one carbon atom and one nitrogen atom as the two points of attachment, the atoms forming part of one or more ring structure(s) wherein at least one of the ring atoms is nitrogen, oxygen or sulfur, and wherein the divalent group consists of no atoms other than carbon, hydrogen, nitrogen, oxygen and sulfur. If more than one ring is present, the rings may be fused or unfused. Unfused rings may be connected via one or more of the following: a covalent bond, alkanediyl, or alkenediyl groups (carbon number limitation permitting). As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkanediyl groups include:

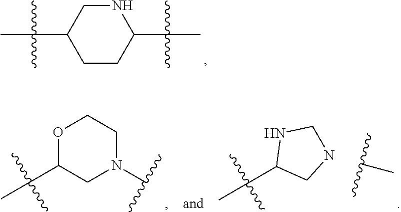

When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), —OC(CH₃)₃(tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkoxydiyl” refers to the divalent group —O-alkanediyl-, —O-alkanediyl-O—, or -alkanediyl-O-alkanediyl-. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃.

The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “alkylaminodiyl” refers to the divalent group —NH-alkanediyl-, —NH-alkanediyl-NH—, or -alkanediyl-NH-alkanediyl-. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±3%, in another example ±1%, and in yet another example ±0.1% from the specified amount, as such variations are appropriate to perform the disclosed method.

As used in this application, the term “average molecular weight” refers to the relationship between the number of moles of each polymer species and the molar mass of that species. In particular, each polymer molecule may have different levels of polymerization and thus a different molar mass. The average molecular weight can be used to represent the molecular weight of a plurality of polymer molecules. Average molecular weight is typically synonymous with average molar mass. In particular, there are three major types of average molecular weight: number average molar mass, weight (mass) average molar mass, and Z-average molar mass. In the context of this application, unless otherwise specified, the average molecular weight represents either the number average molar mass or weight average molar mass of the formula. In some embodiments, the average molecular weight is the number average molar mass. In some embodiments, the average molecular weight may be used to describe a PEG component present in a lipid.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate (e.g., non-human primate). In certain embodiments, the patient or subject is a human. Non-limiting examples of human subjects are adults, juveniles, infants and fetuses.

The term “assemble” or “assembled,” as used herein, in context of delivery of a payload to target cell(s) generally refers to covalent or non-covalent interaction(s) or association(s), for example, such that a therapeutic or prophylactic agent be complexed with or encapsulated in a lipid composition.

As used herein, the term “lipid composition” generally refers to a composition comprising lipid compound(s), including but not limited to, a lipoplex, a liposome, a lipid particle. Examples of lipid compositions include suspensions, emulsions, and vesicular compositions.

As used herein, the term “detectable” refers to an occurrence of, or a change in, a signal that is directly or indirectly detectable either by observation or by instrumentation. Typically, a detectable response is an occurrence of a signal wherein the fluorophore is inherently fluorescent and does not produce a change in signal upon binding to a metal ion or biological compound. Alternatively, the detectable response is an optical response resulting in a change in the wavelength distribution patterns or intensity of absorbance or fluorescence or a change in light scatter, fluorescence lifetime, fluorescence polarization, or a combination of the above parameters. Other detectable responses include, for example, chemiluminescence, phosphorescence, radiation from radioisotopes, magnetic attraction, and electron density

The term “potent” or “potency,” as used herein in connection with delivery of therapeutic agent(s), generally refers to a greater ability of a delivery system (e.g., a lipid composition) to achieve or bring about a desired amount, activity, or effect of a therapeutic agent or prophylactic agent (such as a desired level of translation, transcription, production, expression, or activity of a protein or gene) in cells (e.g., targeted cells) to any measurable extent, e.g., relative to a reference delivery system. For example, a lipid composition with a higher potency may achieve a desired therapeutic effect in a greater population of relevant cells, within a shorter response time, or that last a longer period of time.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present application which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂-]ₙ—, the repeat unit is —CH₂CH₂—. The subscript “n” denotes the degree of polymerization, that is, the number of repeat units linked together. When the value for “n” is left undefined or where “n” is absent, it simply designates repetition of the formula within the brackets as well as the polymeric nature of the material. The concept of a repeat unit applies equally to where the connectivity between the repeat units extends three dimensionally, such as in metal organic frameworks, modified polymers, thermosetting polymers, etc. Within the context of the dendrimer or dendron, the repeating unit may also be described as the branching unit, interior layers, or generations. Similarly, the terminating group may also be described as the surface group.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2″, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present application.

### COMPOSITIONS

### Lipid Compositions

In one aspect, provided herein is a lipid composition comprising: (i) an ionizable cationic lipid; and (iii) a selective organ targeting (SORT) lipid separate from the ionizable cationic lipid. The lipid composition may further comprise a phospholipid.

### Ionizable Cationic Lipids

In some embodiments of the lipid composition of the present application, the lipid composition comprises an ionizable cationic lipid. In some embodiments, the cationic ionizable lipids contain one or more groups which is protonated at physiological pH but may deprotonated and has no charge at a pH above 8, 9, 10, 11, or 12. The ionizable cationic group may contain one or more protonatable amines which are able to form a cationic group at physiological pH. The cationic ionizable lipid compound may also further comprise one or more lipid components such as two or more fatty acids with C₆-C₂₄ alkyl or alkenyl carbon groups. These lipid groups may be attached through an ester linkage or may be further added through a Michael addition to a sulfur atom. In some embodiments, these compounds may be a dendrimer, a dendron, a polymer, or a combination thereof.

In some embodiments of the lipid composition of the present application, the ionizable cationic lipids refer to lipid and lipid-like molecules with nitrogen atoms that can acquire charge (pKa). These lipids may be known in the literature as cationic lipids. These molecules with amino groups typically have between 2 and 6 hydrophobic chains, often alkyl or alkenyl such as C₆-C₂₄ alkyl or alkenyl groups, but may have at least 1 or more that 6 tails. In some embodiments, these cationic ionizable lipids are dendrimers, which are a polymer exhibiting regular dendritic branching, formed by the sequential or generational addition of branched layers to or from a core and are characterized by a core, at least one interior branched layer, and a surface branched layer. (See Petar R. Dvornic and Donald A. Tomalia in Chem. in Britain, 641-645, August 1994.) In other embodiments, the term “dendrimer” as used herein is intended to include, but is not limited to, a molecular architecture with an interior core, interior layers (or “generations”) of repeating units regularly attached to this initiator core, and an exterior surface of terminal groups attached to the outermost generation. A “dendron” is a species of dendrimer having branches emanating from a focal point which is or can be joined to a core, either directly or through a linking moiety to form a larger dendrimer. In some embodiments, the dendrimer structures have radiating repeating groups from a central core which doubles with each repeating unit for each branch. In some embodiments, the dendrimers described herein may be described as a small molecule, medium-sized molecules, lipids, or lipid-like material. These terms may be used to described compounds described herein which have a dendron like appearance (e.g., molecules which radiate from a single focal point).

While dendrimers are polymers, dendrimers may be preferable to traditional polymers because they have a controllable structure, a single molecular weight, numerous and controllable surface functionalities, and traditionally adopt a globular conformation after reaching a specific generation. Dendrimers can be prepared by sequentially reactions of each repeating unit to produce monodisperse, tree-like and/or generational structure polymeric structures. Individual dendrimers consist of a central core molecule, with a dendritic wedge attached to one or more functional sites on that central core. The dendrimeric surface layer can have a variety of functional groups disposed thereon including anionic, cationic, hydrophilic, or lipophilic groups, according to the assembly monomers used during the preparation.

Modifying the functional groups and/or the chemical properties of the core, repeating units, and the surface or terminating groups, their physical properties can be modulated. Some properties which can be varied include, but are not limited to, solubility, toxicity, immunogenicity and bioattachment capability. Dendrimers are often described by their generation or number of repeating units in the branches. A dendrimer consisting of only the core molecule is referred to as Generation 0, while each consecutive repeating unit along all branches is Generation 1, Generation 2, and so on until the terminating or surface group. In some embodiments, half generations are possible resulting from only the first condensation reaction with the amine and not the second condensation reaction with the thiol.

Preparation of dendrimers requires a level of synthetic control achieved through series of stepwise reactions comprising building the dendrimer by each consecutive group. Dendrimer synthesis can be of the convergent or divergent type. During divergent dendrimer synthesis, the molecule is assembled from the core to the periphery in a stepwise process involving attaching one generation to the previous and then changing functional groups for the next stage of reaction. Functional group transformation is necessary to prevent uncontrolled polymerization. Such polymerization would lead to a highly branched molecule that is not monodisperse and is otherwise known as a hyperbranched polymer. Due to steric effects, continuing to react dendrimer repeat units leads to a sphere shaped or globular molecule, until steric overcrowding prevents complete reaction at a specific generation and destroys the molecule's monodispersity. Thus, in some embodiments, the dendrimers of G1-G10 generation are specifically contemplated. In some embodiments, the dendrimers comprise 1, 2, 3, 4, 5, 6, 7, 8, 9, or 10 repeating units, or any range derivable therein. In some embodiments, the dendrimers used herein are G0, G1, G2, or G3. However, the number of possible generations (such as 11, 12, 13, 14, 15, 20, or 25) may be increased by reducing the spacing units in the branching polymer.

Additionally, dendrimers have two major chemical environments: the environment created by the specific surface groups on the termination generation and the interior of the dendritic structure which due to the higher order structure can be shielded from the bulk media and the surface groups. Because of these different chemical environments, dendrimers have found numerous different potential uses including in therapeutic applications.

In some embodiments of the lipid composition of the present application, the dendrimers or dendrons are assembled using the differential reactivity of the acrylate and methacrylate groups with amines and thiols. The dendrimers or dendrons may include secondary or tertiary amines and thioethers formed by the reaction of an acrylate group with a primary or secondary amine and a methacrylate with a mercapto group. Additionally, the repeating units of the dendrimers or dendrons may contain groups which are degradable under physiological conditions. In some embodiments, these repeating units may contain one or more germinal diethers, esters, amides, or disulfides groups. In some embodiments, the core molecule is a monoamine which allows dendritic polymerization in only one direction. In other embodiments, the core molecule is a polyamine with multiple different dendritic branches which each may comprise one or more repeating units. The dendrimer or dendron may be formed by removing one or more hydrogen atoms from this core. In some embodiments, these hydrogen atoms are on a heteroatom such as a nitrogen atom. In some embodiments, the terminating group is a lipophilic group such as a long chain alkyl or alkenyl group. In other embodiments, the terminating group is a long chain haloalkyl or haloalkenyl group. In other embodiments, the terminating group is an aliphatic or aromatic group containing an ionizable group such as an amine (—NH₂) or a carboxylic acid (—CO₂H). In still other embodiments, the terminating group is an aliphatic or aromatic group containing one or more hydrogen bond donors such as a hydroxide group, an amide group, or an ester.

The cationic ionizable lipids of the present application may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Cationic ionizable lipids may occur as racemates and racemic mixtures, single enantiomers, diastereomeric mixtures and individual diastereomers. In some embodiments, a single diastereomer is obtained. The chiral centers of the cationic ionizable lipids of the present application can have the S or the R configuration. Furthermore, it is contemplated that one or more of the cationic ionizable lipids may be present as constitutional isomers. In some embodiments, the compounds have the same formula but different connectivity to the nitrogen atoms of the core. Without wishing to be bound by any theory, it is believed that such cationic ionizable lipids exist because the starting monomers react first with the primary amines and then statistically with any secondary amines present. Thus, the constitutional isomers may present the fully reacted primary amines and then a mixture of reacted secondary amines.

Chemical formulas used to represent cationic ionizable lipids of the present application will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given formula, and regardless of which one is most prevalent, all tautomers of a given chemical formula are intended.

The cationic ionizable lipids of the present application may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the cationic ionizable lipids of the present application are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

It should be recognized that the particular anion or cation forming a part of any salt form of a cationic ionizable lipids provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

In some embodiments of the lipid composition of the present application, the ionizable cationic lipid is a dendrimer or dendron. In some embodiments, the ionizable cationic lipid comprises an ammonium group which is positively charged at physiological pH and contains at least two hydrophobic groups. In some embodiments, the ammonium group is positively charged at a pH from about 6 to about 8. In some embodiments, the ionizable cationic lipid is a dendrimer or dendron. In some embodiments, the ionizable cationic lipid comprises at least two C₆-C₂₄ alkyl or alkenyl groups.

### Dendrimers or Dendrons of Formula (I)

In some embodiments of the lipid composition, the ionizable cationic lipid comprises at least two C₈-C₂₄ alkyl groups. In some embodiments, the ionizable cationic lipid is a dendrimer or dendron further defined by the formula:

### Core-Repeating Unit-Terminating Group (D-I)

wherein the core is linked to the repeating unit by removing one or more hydrogen atoms from the core and replacing the atom with the repeating unit and wherein:
   the core has the formula:

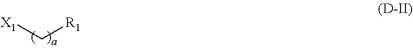

wherein:
   X₁ is amino or alkylamino_{(C≤12)}, dialkylamino_{(C≤12)}, heterocycloalkyl_{(C≤12)}, heteroaryl_{(C≤12)}, or a substituted version thereof,
   R₁ is amino, hydroxy, or mercapto, or alkylamino_{(C≤12)}, dialkylamino_{(C≤12)}, or a substituted version of either of these groups; and
   a is 1, 2, 3, 4, 5, or 6; or
the core has the formula:

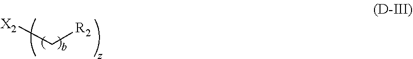

wherein:
   X₂ is N(R₅)_{y};
   R₅ is hydrogen, alkyl_{(C≤18)}, or substituted alkyl_{(C≤18)}; and
   y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3; or
the core has the formula:

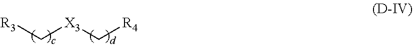

wherein:
   X₃ is —NR₆—, wherein R₆ is hydrogen, alkyl_{(C≤8)}, or substituted alkyl_{(C≤8)}, —O—, or alkylaminodiyl_{(C≤8)}, alkoxydiyl_{(C≤8)}, arenediyl_{(C≤18)}, heteroarenediyl_{(C≤18)}, heterocycloalkanediyl_{(C≤8)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

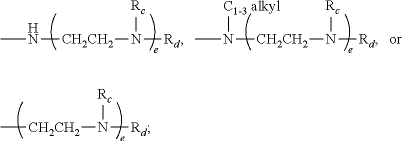

wherein:
e and f are each independently 1, 2, or 3; provided that the sum of e and f is 3;
R_{c}, R_{d}, and R_{f} are each independently hydrogen, alkyl_{(C≤6)}, or substituted alkyl_{(C≤6)};
c and d are each independently 1, 2, 3, 4, 5, or 6; or
the core is alkylamine_{(C≤18)}, dialkylamine_{(C≤36)}, heterocycloalkane_{(C≤12)}, or a substituted version of any of these groups;
wherein the repeating unit comprises a degradable diacyl and a linker;
   the degradable diacyl group has the formula:

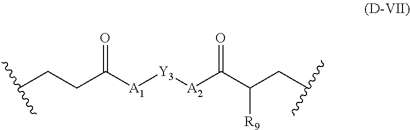

wherein:
   A₁ and A₂ are each independently —O—, —S—, or —NRₐ—, wherein:
   Rₐ is hydrogen, alkyl_{(C≤6)}, or substituted alkyl_{(C≤6)};
   Y₃ is alkanediyl_{(C≤12)}, alkenediyl_{(C≤12)}, arenediyl_{(C≤12)}, or a substituted version of any of these groups; or a group of the formula:

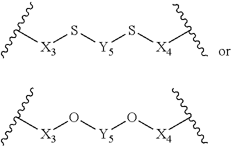

wherein:
X₃ and X₄ are alkanediyl_{(C≤12)}, alkenediyl_{(C≤12)}, arenediyl_{(C≤12)}, or a substituted version of any of these groups;
Y₅ is a covalent bond, alkanediyl_{(C≤12)}, alkenediyl_{(C≤12)}, arenediyl_{(C≤12)}, or a substituted version of any of these groups; and
R₉ is alkyl_{(C≤18)} or substituted alkyl_{(C≤18)};
the linker group has the formula:

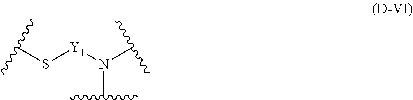

wherein:
   Y₁ is alkanediyl_{(C≤12)}, alkenediyl_{(C≤12)}, arenediyl_{(C≤12)}, or a substituted version of any of these groups; and
wherein when the repeating unit comprises a linker group, then the linker group comprises an independent degradable diacyl group attached to both the nitrogen and the sulfur atoms of the linker group if n is greater than 1, wherein the first group in the repeating unit is a degradable diacyl group, wherein for each linker group, the next repeating unit comprises two degradable diacyl groups attached to the nitrogen atom of the linker group; and wherein n is the number of linker groups present in the repeating unit; and
the terminating group has the formula:

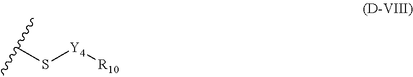

wherein:
   Y₄ is alkanediyl_{(C≤18)} or an alkanediyl_{(C≤18)} wherein one or more of the hydrogen atoms on the alkanediyl_{(C≤18)} has been replaced with —OH, —F, —Cl, —Br, —I, —SH, —OCH₃, —OCH₂CH₃, —SCH₃, or —OC(O)CH₃;
   R₁₀ is hydrogen, carboxy, hydroxy, or
   aryl_{(C≤12)}, alkylamino_{(C≤12)}, dialkylamino_{(C≤12)}, N-heterocycloalkyl_{(C≤12)}, —C(O)N(R₁₁)-alkanediyl_{(C≤6)}-heterocycloalkyl_{(C≤12)}, —C(O)-alkylamino_{(C≤12)}, —C(O)-dialkylamino_{(C≤12)}, —C(O)—N-heterocycloalkyl_{(C≤12)}, wherein:
   R₁₁ is hydrogen, alkyl_{(C≤6)}, or substituted alkyl_{(C≤6)};
   wherein the final degradable diacyl in the chain is attached to a terminating group;
   n is 0, 1, 2, 3, 4, 5, or 6;
or a pharmaceutically acceptable salt thereof. In some embodiments, the terminating group is further defined by the formula:

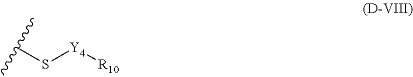

wherein:
   Y₄ is alkanediyl_{(C≤18)}; and
   R₁₀ is hydrogen. In some embodiments, A₁ and A₂ are each independently —O— or —NRₐ—.

In some embodiments of the dendrimer or dendron of formula (D-I), the core is further defined by the formula:

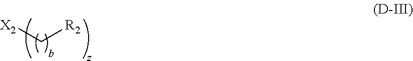

wherein:
   X₂ is N(R₅)_{y};
      R₅ is hydrogen or alkyl_{(C≤18)}, or substituted alkyl_{(C≤18)}; and
      y is 0, 1, or 2, provided that the sum of y and z is 3;
   R₂ is amino, hydroxy, or mercapto, or alkylamino_{(C≤12)}, dialkylamino_{(C≤12)}, or a substituted version of either of these groups;
   b is 1, 2, 3, 4, 5, or 6; and
   z is 1, 2, 3; provided that the sum of z and y is 3.

In some embodiments of the dendrimer or dendron of formula (D-I), the core is further defined by the formula:

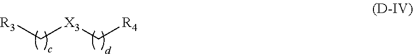

wherein:
   X₃ is —NR₆—, wherein R₆ is hydrogen, alkyl_{(C≤18)}, or substituted alkyl_{(C≤18)}, —O—, or alkylaminodiyl_{(C≤18)}, alkoxydiyl_{(C≤18)}, arenediyl_{(C≤18)}, heteroarenediyl_{(C≤18)}, heterocycloalkanediyl_{(C≤18)}, or a substituted version of any of these groups;
   R₃ and R₄ are each independently amino, hydroxy, or mercapto, or alkylamino_{(C≤12)}, dialkylamino_{(C≤12)}, or a substituted version of either of these groups; or a group of the formula: —N(R_{f})_{f}(CH₂CH₂N(R_{c}))ₑR_{d},

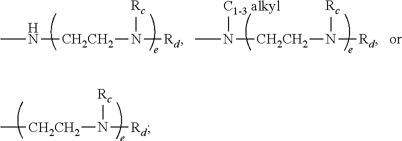

wherein:
   e and f are each independently 1, 2, or 3; provided that the sum of e and f is 3;
   Rₑ, R_{d}, and R_{f} are each independently hydrogen, alkyl_{(C≤6)}, or substituted alkyl_{(C≤6)};
c and d are each independently 1, 2, 3, 4, 5, or 6.

In some embodiments of the dendrimer or dendron of formula (I), the terminating group is represented by the formula:

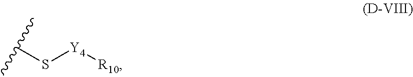

wherein:
Y₄ is alkanediyl_{(C≤18)}; and
R₁₀ is hydrogen.

In some embodiments of the dendrimer or dendron of formula (D-I), the core is further defined as:

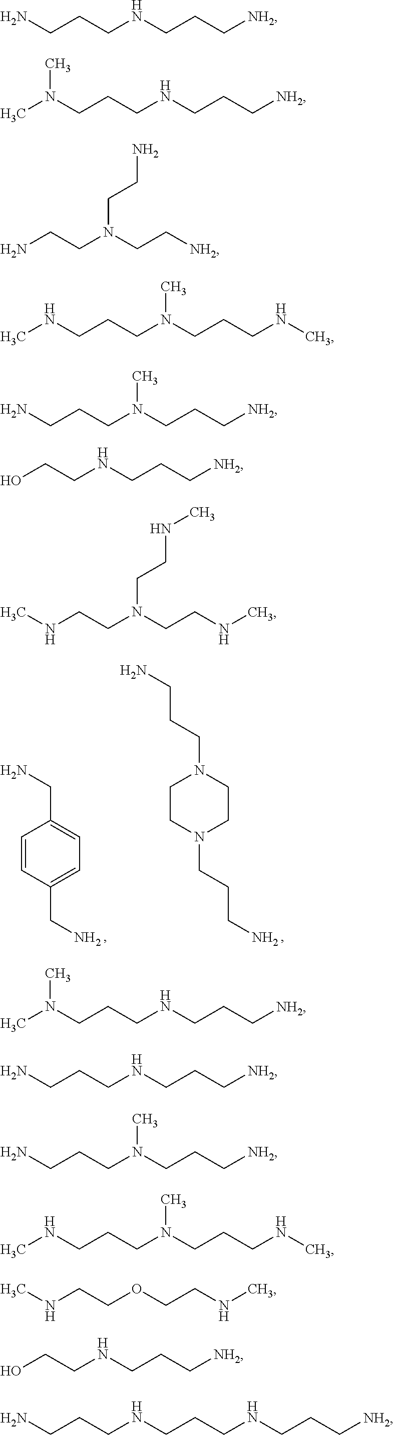

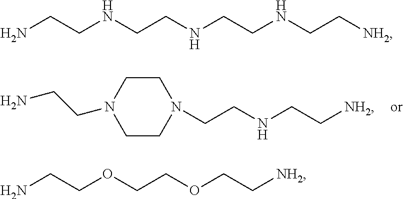

In some embodiments of the dendrimer or dendron of formula (D-I), the degradable diacyl is further defined as:

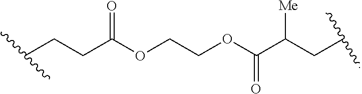

In some embodiments of the dendrimer or dendron of formula (D-I), the linker is further defined as

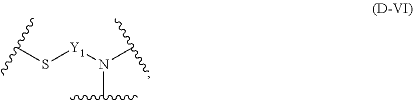

wherein Y₁ is alkanediyl_{(C≤18)} or substituted alkanediyl_{(C≤18)}.

In some embodiments of the dendrimer or dendron of formula (D-I), the dendrimer or dendron is selected from the group consisting of:

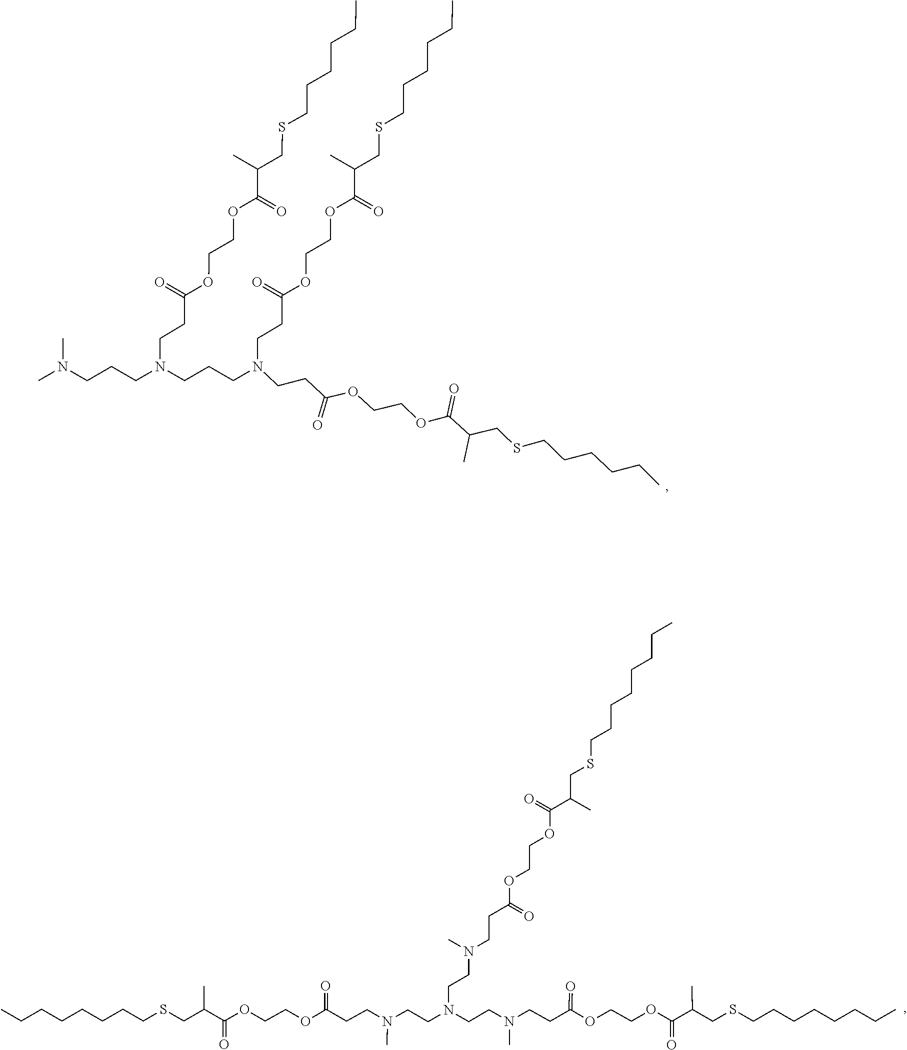

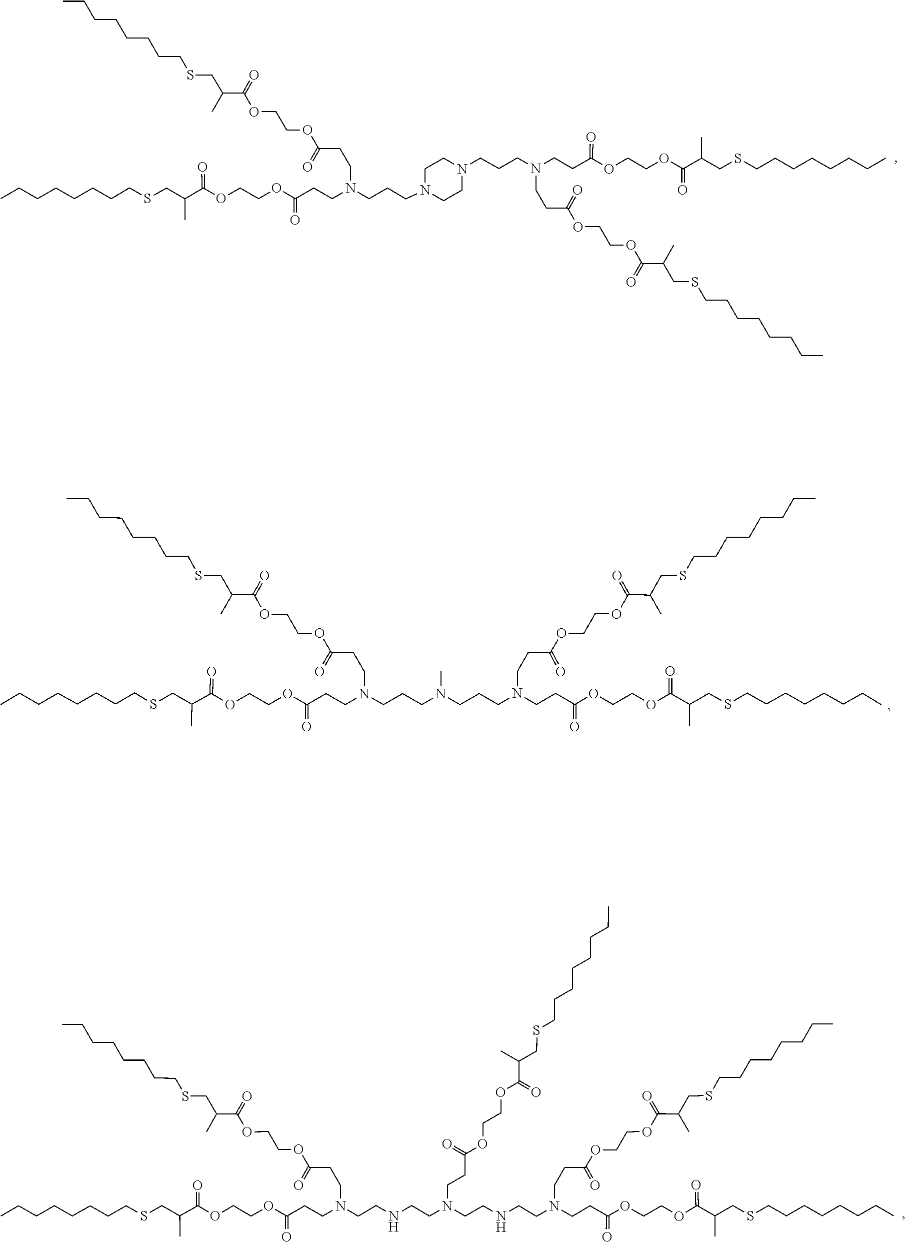

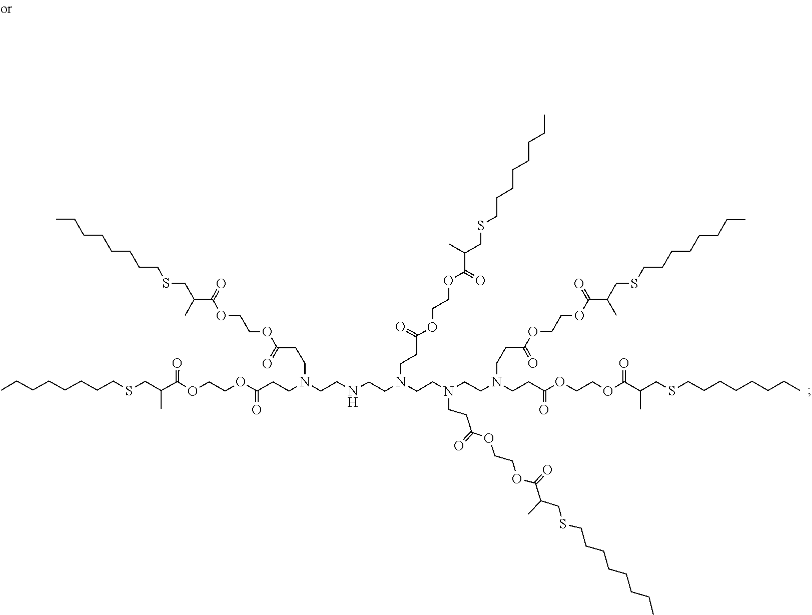 [SMILES: CCCCCCCCSCC(C)C(=O)OCCOC(=O)CCN(CCNCCN(CCC(=O)OCCOC(=O)C(C)CSCCCCCCCC)CCN(CCC(=O)OCCOC(=O)C(C)CSCCCCCCCC)CCN(CCC(=O)OCCOC(=O)C(C)CSCCCCCCCC)CCC(=O)OCCOC(=O)C(C)CSCCCCCCCC)CCC(=O)OCCOC(=O)C(C)CSCCCCCCCC]

and pharmaceutically acceptable salts thereof.

### Dendrimers or Dendrons of Formula (X)

In some embodiments of the lipid composition, the ionizable cationic lipid is a dendrimer or dendron of the formula Core [symbol] Branch)_{N}. In some embodiments, the ionizable cationic lipid is a dendrimer or dendron of the formula

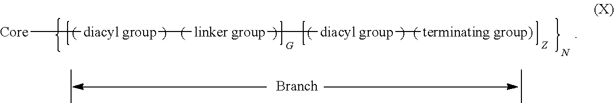

In some embodiments of the lipid composition, the ionizable cationic lipid is a dendrimer or dendron of a generation (g) having a structural formula:

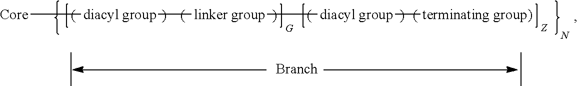

or a pharmaceutically acceptable salt thereof, wherein:
(a) the core comprises a structural formula (X_{Core}):

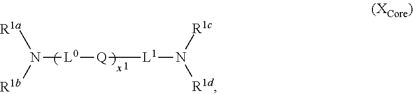

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

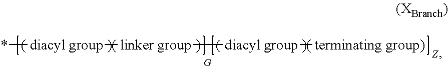

wherein:
   * indicates a point of attachment of the branch to the core;
   g is 1, 2, 3, or 4;
   Z=2^{(g-1)};
   G=0, when g=1; or G=Σᵢ₌₀^{i=g-2}, when g≠1;
(c) each diacyl group independently comprises a structural formula

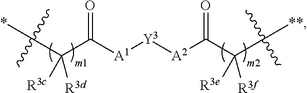

wherein:
   * indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

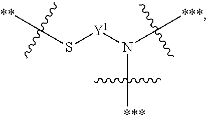

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

In some embodiments of X_{Core}, Q is independently at each occurrence a covalent bond, —O—, —S—, —NR²—, or —CR^{3a}R^{3b}. In some embodiments of X_{Core} Q is independently at each occurrence a covalent bond.

In some embodiments of X_{Core} Q is independently at each occurrence an —O—. In some embodiments of X_{Core} Q is independently at each occurrence a —S—. In some embodiments of X_{Core} Q is independently at each occurrence a —NR² and R² is independently at each occurrence R^{1g} or -L²-NR^{1e}R^{1f}. In some embodiments of X_{Core} Q is independently at each occurrence a —CR^{3a}R^{3b}R^{3a}, and R^{3a} and R^{3b} are each independently at each occurrence hydrogen or an optionally substituted alkyl (e.g., C₁-C₆, such as C₁-C₃).

In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen, or an optionally substituted alkyl. In some embodiments of X_{Core}, R^{1a}, R^{1b}, R^{1c}, R^{1d}, R^{1e}, R^{1f}, and R^{1g} (if present) are each independently at each occurrence a point of connection to a branch, hydrogen. In some embodiments of X_{Core}, R^{1a}, R^{1b}, R^{1c}, R^{1d}, R^{1e}, R^{1f}, and R^{1g} (if present) are each independently at each occurrence a point of connection to a branch an optionally substituted alkyl (e.g., C₁-C₁₂).

In some embodiments of X_{Core}, L⁰, L¹, and L² are each independently at each occurrence selected from a covalent bond, alkylene, heteroalkylene, [alkylene]-[heterocycloalkyl]-[alkylene], [alkylene]-(arylene)-[alkylene], heterocycloalkyl, and arylene; or, alternatively, part of L¹ form a heterocycloalkyl (e.g., C₄-C₆ and containing one or two nitrogen atoms and, optionally, an additional heteroatom selected from oxygen and sulfur) with one of R^{1c} and R^{1d}. In some embodiments of X_{Core}, L⁰, L¹, and L² are each independently at each occurrence can be a covalent bond. In some embodiments of X_{Core}, L⁰, L¹, and L² are each independently at each occurrence can be a hydrogen. In some embodiments of X_{Core}, L⁰, L¹, and L² are each independently at each occurrence can be an alkylene (e.g., C₁-C₁₂, such as C₁-C₆ or C₁-C₃). In some embodiments of X_{Core}, L⁰, L¹, and L² are each independently at each occurrence can be a heteroalkylene (e.g., C₁-C₁₂, such as C₁-C₈ or C₁-C₆). In some embodiments of X_{Core}, L⁰, L¹, and L² are each independently at each occurrence can be a heteroalkylene (e.g., C₂-C₈ alkyleneoxide, such as oligo(ethyleneoxide)). In some embodiments of X_{Core}, L⁰, L¹, and L² are each independently at each occurrence can be a [alkylene]-[heterocycloalkyl]-[alkylene] [(e.g., C₁-C₆) alkylene]-[(e.g., C₄-C₆) heterocycloalkyl]-[(e.g., C₁-C₆) alkylene]. In some embodiments of X_{Core}, L⁰, L¹, and L² are each independently at each occurrence can be a [alkylene]-(arylene)-[alkylene] [(e.g., C₁-C₆) alkylene]-(arylene)-[(e.g., C₁-C₆) alkylene]. In some embodiments of X_{Core}, L⁰, L¹, and L² are each independently at each occurrence can be a [alkylene]-(arylene)-[alkylene] (e.g., [(e.g., C₁-C₆) alkylene]-phenylene-[(e.g., C₁-C₆) alkylene]). In some embodiments of X_{Core}, L⁰, L¹, and L² are each independently at each occurrence can be a heterocycloalkyl (e.g., C₄-C₆heterocycloalkyl). In some embodiments of X_{Core}, L⁰, L¹, and L² are each independently at each occurrence can be an arylene (e.g., phenylene). In some embodiments of X_{Core}, part of L¹ form a heterocycloalkyl with one of R^{1c} and R^{1d}. In some embodiments of X_{Core}, part of L¹ form a heterocycloalkyl (e.g., C₄-C₆ heterocycloalkyl) with one of R^{1c} and R^{1d} and the heterocycloalkyl can contain one or two nitrogen atoms and, optionally, an additional heteroatom selected from oxygen and sulfur.

In some embodiments of X_{Core}, L⁰, L¹, and L² are each independently at each occurrence selected from a covalent bond, C₁-C₆ alkylene (e.g., C₁-C₃ alkylene), C₂-C₁₂ (e.g., C₂-C₅) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene] (e.g.,

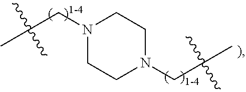

and [(C₁-C₄) alkylene]-phenylene-[(C₁-C₄) alkylene] (e.g.,

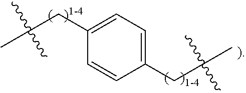

In some embodiments of X_{Core}, L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-. In some embodiments of X_{Core}, L⁰, L¹, and L² are each independently at each occurrence C₁-C₆ alkylene (e.g., C₁-C₃ alkylene). In some embodiments, L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)). In some embodiments of X_{Core}, L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

In some embodiments of X_{Core}, x¹ is 0, 1, 2, 3, 4, 5, or 6. In some embodiments of X_{Core}, x¹ is 0. In some embodiments of X_{Core}, x¹ is 1. In some embodiments of X_{Core}, x¹ is 2. In some embodiments of X_{core}, x¹ is 0, 3. In some embodiments of X_{Core}, x¹ is 4. In some embodiments of X_{Core} x¹ is 5. In some embodiments of X_{Core}, x¹ is 6.

In some embodiments of X_{Core}, the core comprises a structural formula:

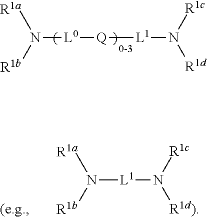

In some embodiments of X_{Core}, the core comprises a structural formula:

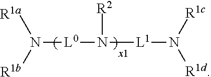

In some embodiments of X_{Core}, the core comprises a structural formula:

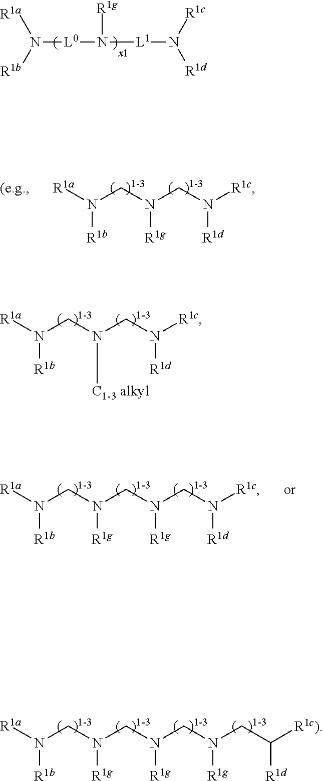

In some embodiments of X_{Core}, the core comprises a structural formula:

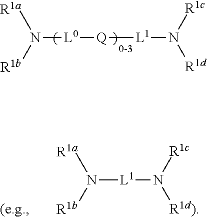

In some embodiments of X_{Core}, the core comprises a structural formula:

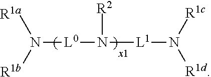

In some embodiments of X_{Core}, the core comprises a structural formula:

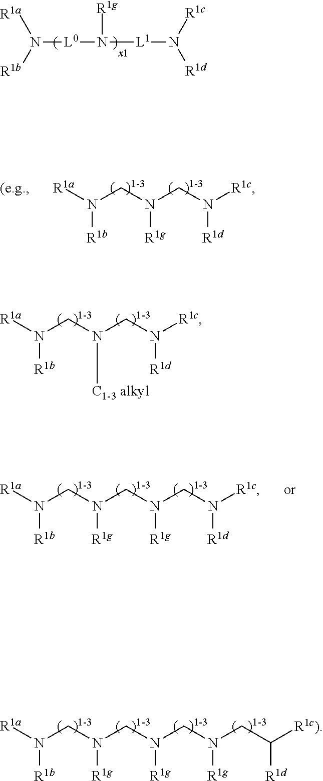

In some embodiments of X_{core}, the core comprises a structural formula:

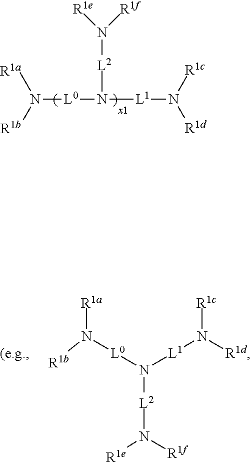

such as

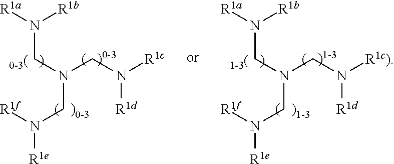

In some embodiments of X_{Core}, the core comprises a structural formula:

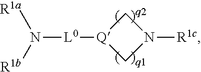

wherein Q′ is —NR²— or —CR^{3a}R^{3b}—; q¹ and q² are each independently 1 or 2. In some embodiments of X_{Core}, the core comprises a structural formula:

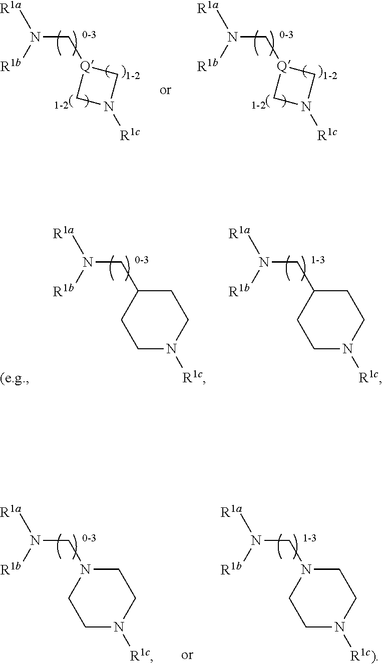

In some embodiments of X_{Core}, the core comprises a structural formula

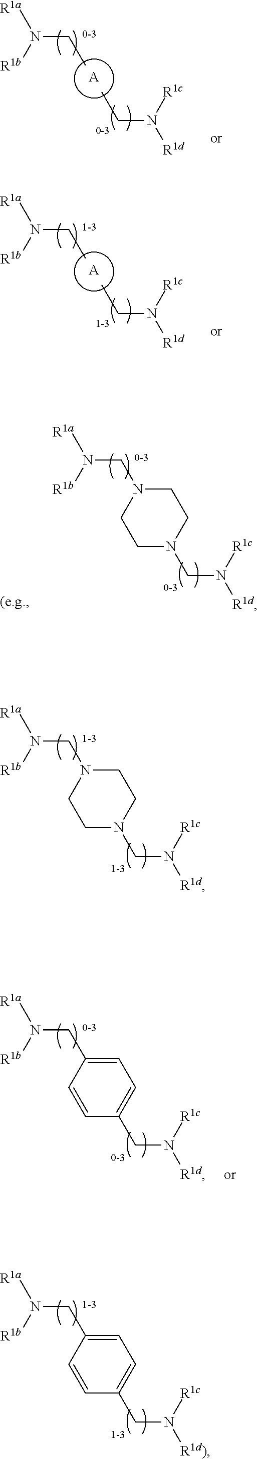

wherein ring A is an optionally substituted aryl or an optionally substituted (e.g., C₃-C₁₂, such as C₃-C₅) heteroaryl. In some embodiments of X_{Core}, the core comprises has a structural formula

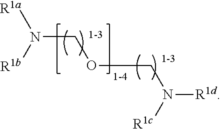

In some embodiments of X_{Core}, the core comprises a structural formula set forth in Table. 1 and pharmaceutically acceptable salts thereof, wherein * indicates a point of attachment of the core to a branch of the plurality of branches. In some embodiments, the example cores of Table. 1 are not limited to the stereoisomers (i.e., enantiomers, diastereomers) listed.

TABLE 1
Example core structures

| ID # | Structure |
|---|---|
| 1A1 | 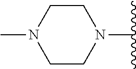 [SMILES: *N1CCN(C)CC1] |
| 1A2-1 | 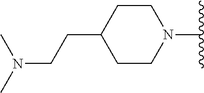 [SMILES: *N1CCC(CCN(C)C)CC1] |
| 1A2-2 | 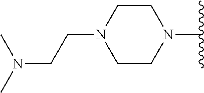 [SMILES: *N1CCN(CCN(C)C)CC1] |
| 1A3-1 | 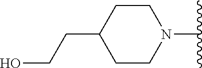 [SMILES: *N1CCC(CCO)CC1] |
| 1A3-2 | 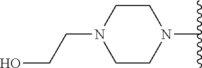 [SMILES: *N1CCN(CCO)CC1] |
| 1A4 | 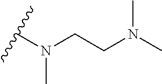 [SMILES: *N(C)CCN(C)C] |
| 1A5-1 | 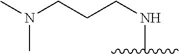 [SMILES: *NCCCN(C)C] |
| 1A-2 | 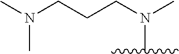 |
| 2A1-1 | 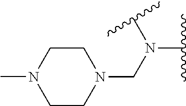 [SMILES: *N(*)CN1CCN(C)CC1] |
| 2A1-2 | 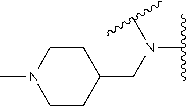 [SMILES: *N(*)CC1CCN(C)CC1] |
| 2A2-1 | 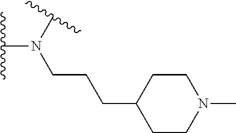 [SMILES: *N(*)CCCC1CCN(C)CC1] |
| 2A2-2 | 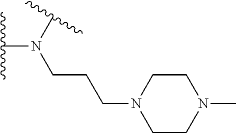 [SMILES: *N(*)CCCN1CCN(C)CC1] |
| 2A3 | 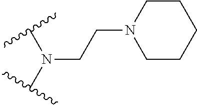 [SMILES: *N(*)CCN1CCCCC1] |
| 2A4 | 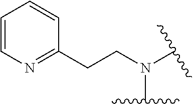 [SMILES: *N(*)CCc1ccccn1] |
| 2A5 | 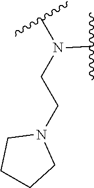 [SMILES: *N(*)CCN1CCCC1] |
| 2A6 | 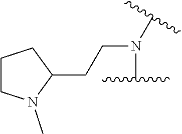 [SMILES: *N(*)CCC1CCCN1C] |
| 2A7-1 | 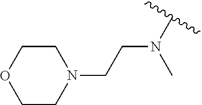 [SMILES: *N(C)CCN1CCOCC1] |
| 2A7-2 | 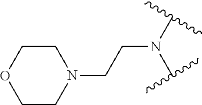 [SMILES: *N(*)CCN1CCOCC1] |
| 2A8 | 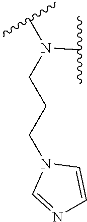 [SMILES: *N(*)CCCn1ccnc1] |
| 2A9 | 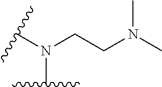 [SMILES: *N(*)CCN(C)C] |
| 2A9V | 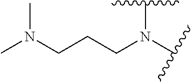 [SMILES: *N(*)CCCN(C)C] |
| 2A10 | 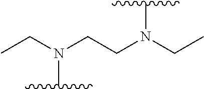 [SMILES: *N(CC)CCN(*)CC] |
| 2A11 | 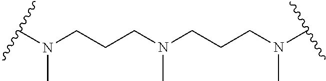 [SMILES: *N(C)CCCN(C)CCCN(*)C] |
| 2A12 | 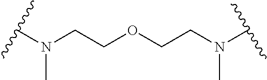 [SMILES: *N(C)CCOCCN(*)C] |
| 3A1 | 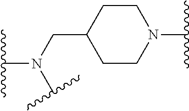 [SMILES: *N(*)CC1CCN(*)CC1] |
| 3A2 | 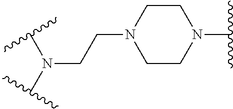 [SMILES: *N(*)CCN1CCN(*)CC1] |
| 3A3 | 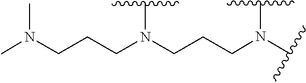 [SMILES: *N(*)CCCN(*)CCCN(C)C] |
| 3A4 | 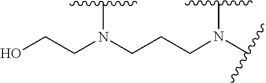 [SMILES: *N(*)CCCN(*)CCO] |
| 3A5 | 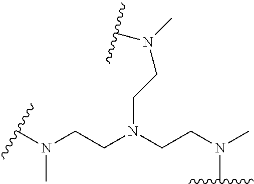 [SMILES: *N(C)CCN(CCN(*)C)CCN(*)C] |
| 3A6 | 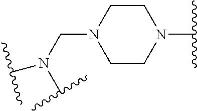 [SMILES: *N(*)CN1CCN(*)CC1] |
| 3A7 | 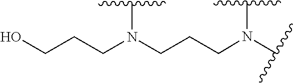 [SMILES: *N(*)CCCN(*)CCCO] |
| 4A1 | 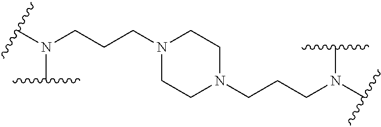 [SMILES: *N(*)CCCN1CCN(CCCN(*)*)CC1] |
| 4A2 | 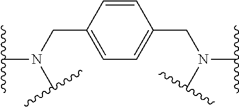 [SMILES: *N(*)Cc1ccc(CN(*)*)cc1] |
| 4A3 | 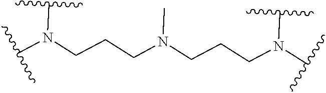 [SMILES: *N(*)CCCN(C)CCCN(*)*] |
| 4A4 | 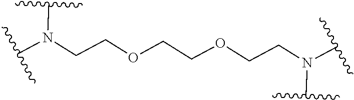 [SMILES: *N(*)CCOCCOCCN(*)*] |
| 5A1 | 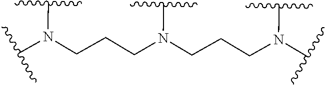 [SMILES: *N(*)CCCN(*)CCCN(*)*] |
| 5A2-1 (5-arm) | 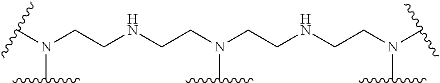 [SMILES: *N(*)CCNCCN(*)CCNCCN(*)*] |
| 5A2-2 (5-arm) | 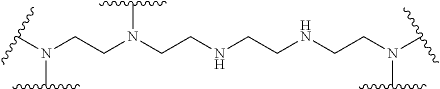 [SMILES: *N(*)CCNCCNCCN(*)CCN(*)*] |
| 5A2-3 (5-arm) | 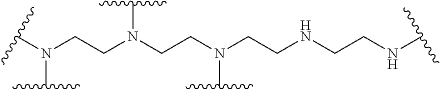 [SMILES: *NCCNCCN(*)CCN(*)CCN(*)*] |
| 5A2-4 (5-arm) | 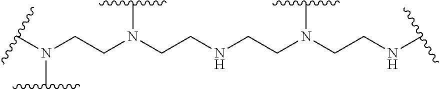 [SMILES: *NCCN(*)CCNCCN(*)CCN(*)*] |
| 5A3-1 (5-arm) | 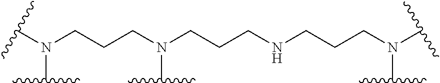 [SMILES: *N(*)CCCNCCCN(*)CCCN(*)*] |
| 5A4-1 (5 arm) | 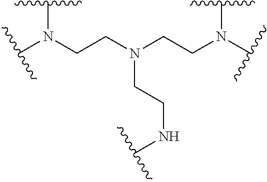 [SMILES: *NCCN(CCN(*)*)CCN(*)*] |
| 5A5 | 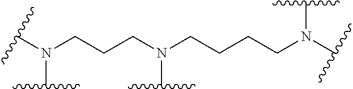 [SMILES: *N(*)CCCCN(*)CCCN(*)*] |
| 5A6 | 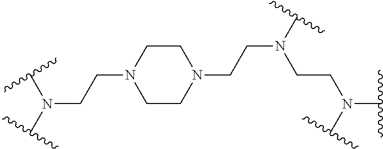 [SMILES: *N(*)CCN(*)CCN1CCN(CCN(*)*)CC1] |
| 5A2-4 (6 arm) | 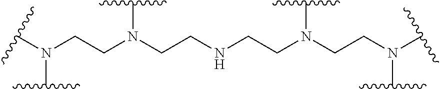 [SMILES: *N(*)CCN(*)CCNCCN(*)CCN(*)*] |
| 5A2-5 (6 arm) | 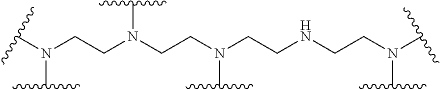 [SMILES: *N(*)CCNCCN(*)CCN(*)CCN(*)*] |
| 5A2-6 (6 arm) | 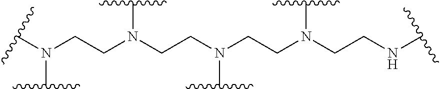 [SMILES: *NCCN(*)CCN(*)CCN(*)CCN(*)*] |
| 5A3-2 (6 arm) | 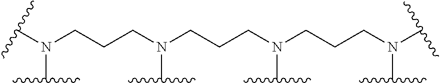 [SMILES: *N(*)CCCN(*)CCCN(*)CCCN(*)*] |
| 5A4-2 (6 arm) | 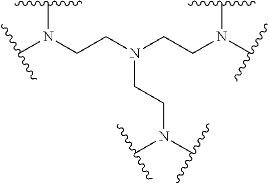 [SMILES: *N(*)CCN(CCN(*)*)CCN(*)*] |
| 6A4 | 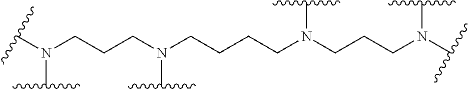 [SMILES: *N(*)CCCN(*)CCCCN(*)CCCN(*)*] |
| 1H1 | 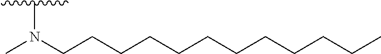 [SMILES: *N(C)CCCCCCCCCCCC] |
| 1H2 | 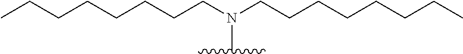 [SMILES: *N(CCCCCCCC)CCCCCCCC] |
| 1H3 | 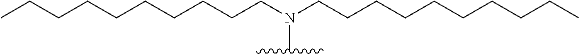 [SMILES: *N(CCCCCCCCCC)CCCCCCCCCC] |
| 2H1 | 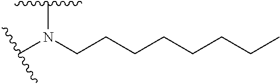 [SMILES: *N(*)CCCCCCCC] |
| 2H2 | 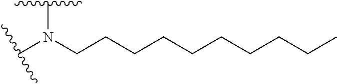 [SMILES: *N(*)CCCCCCCCCC] |
| 2H3 | 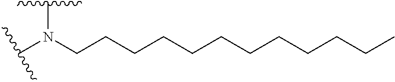 [SMILES: *N(*)CCCCCCCCCCCC] |
| 2H4 | 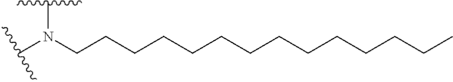 [SMILES: *N(*)CCCCCCCCCCCCCC] |
| 2H5 | 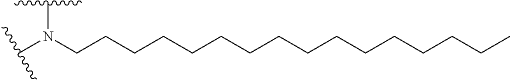 [SMILES: *N(*)CCCCCCCCCCCCCCCC] |
| 2H6 | 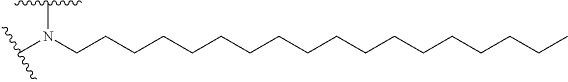 [SMILES: *N(*)CCCCCCCCCCCCCCCCCC] |

In some embodiments of X_{Core}, the core comprises a structural formula selected from the group consisting of:

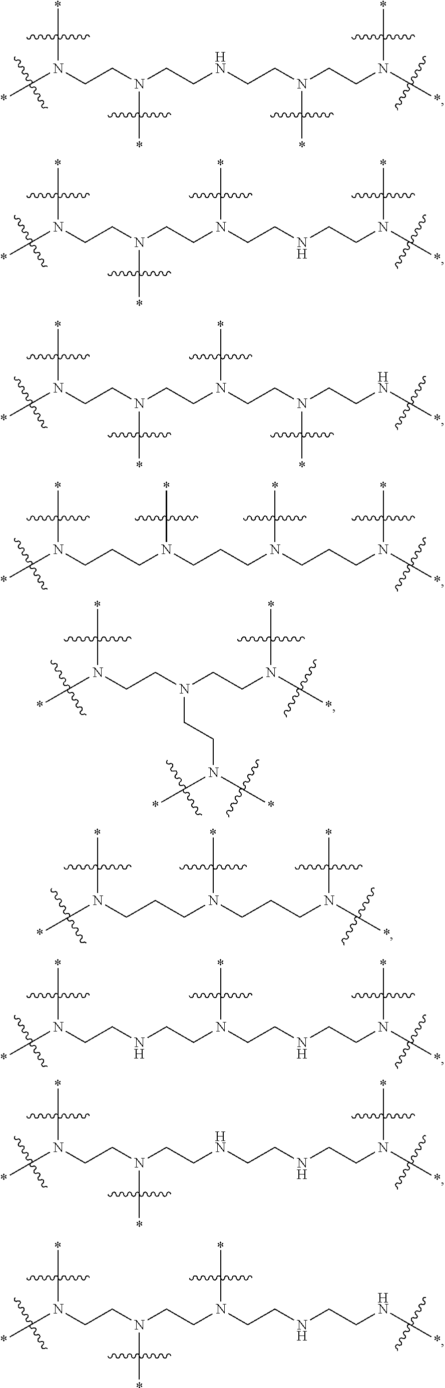

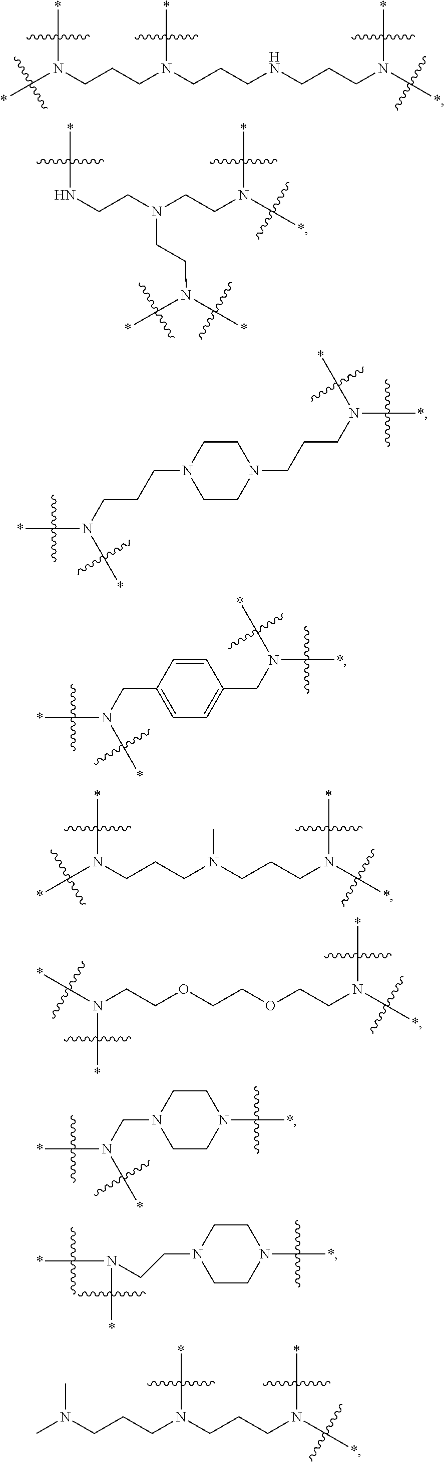

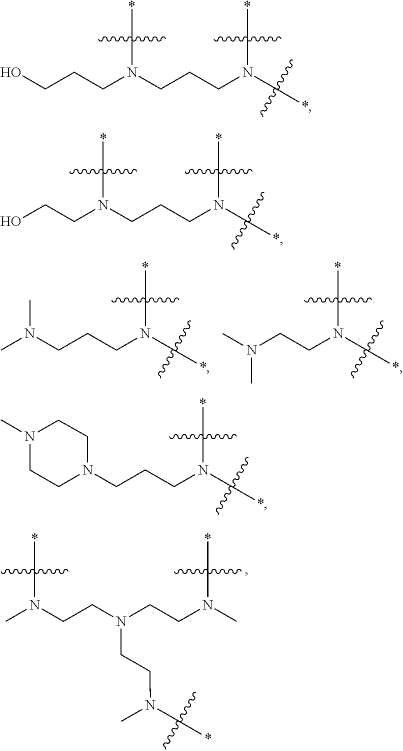

and pharmaceutically acceptable salts thereof, wherein * indicates a point of attachment of the core to a branch of the plurality of branches or H. In some embodiments, wherein * indicates a point of attachment of the core to a branch of the plurality of branches.

In some embodiments of X_{Core}, the core has the structure

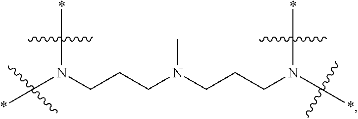

wherein * indicates a point of attachment of the core to a branch of the plurality of branches or H. In some embodiments, at least 2 branches are attached to the core. In some embodiments, at least 3 branches are attached to the core. In some embodiments, at least 4 branches are attached to the core.

In some embodiments of X_{Core}, the core has the structure

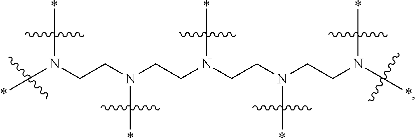

wherein * indicates a point of attachment of the core to a branch of the plurality of branches or H. In some embodiments, at least 4 branches are attached to the core. In some embodiments, at least 5 branches are attached to the core. In some embodiments, at least 6 branches are attached to the core.

In some embodiments, the plurality (N) of branches comprises at least 3 branches, at least 4 branches, at least 5 branches. In some embodiments, the plurality (N) of branches comprises at least 3 branches. In some embodiments, the plurality (N) of branches comprises at least 4 branches. In some embodiments, the plurality (N) of branches comprises at least 5 branches.

In some embodiments of X_{Branch}, g is 1, 2, 3, or 4. In some embodiments of X_{Brach}, g is 1. In some embodiments of X_{Branch}, g is 2. In some embodiments of X_{Branch}, g is 3. In some embodiments of X_{Branch}, g is 4.

In some embodiments of X_{Branch}, Z=2^{(g-1)} and when g=1, G=0. In some embodiments of X_{Branch}, Z=2^{(g-1)} and G=Σᵢ₌₁^{i=g-2}2ⁱ, when g≠1.

In some embodiments of X_{Branch}, g=1, G=0, Z=1, and each branch of the plurality of branches comprises a structural formula each branch of the plurality of branches comprises a structural formula

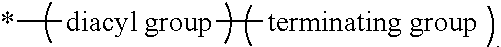

In some embodiments of X_{Branch}, g=2, G=1, Z=2, and each branch of the plurality of branches comprises a structural formula

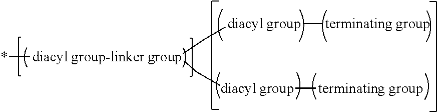

In some embodiments of X_{Branch}, g=3, G=3, Z=4, and each branch of the plurality of branches comprises a structural formula

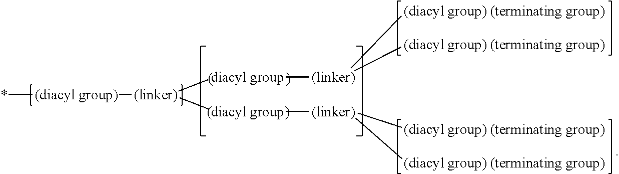

In some embodiments of X_{Branch}, g=4, G=7, Z=8, and each branch of the plurality of branches comprises a structural formula

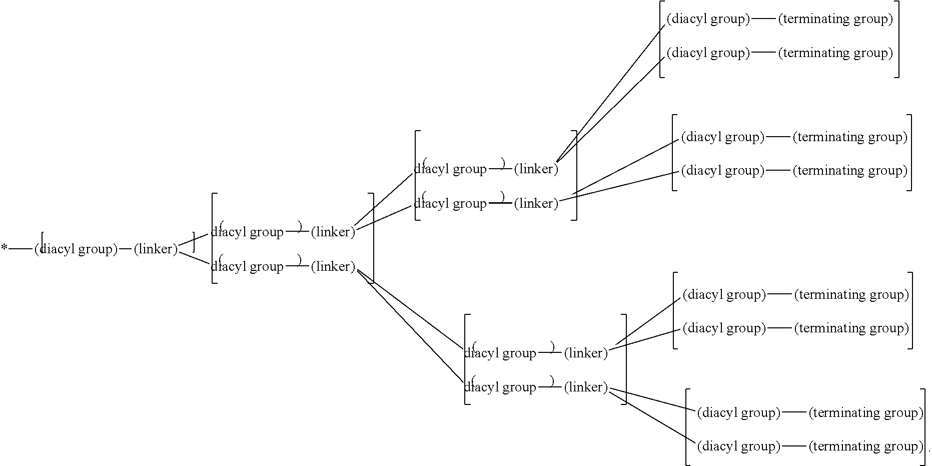

In some embodiments, the dendrimers or dendrons described herein with a generation (g)=1 has the structure:

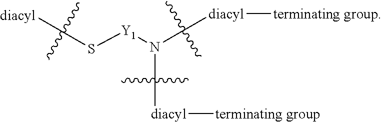

In some embodiments, the dendrimers or or dendrons described herein with a generation (g)=1 has the structure:

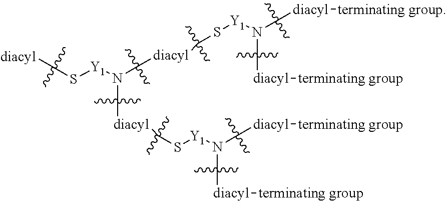

An example formulation of the dendrimers or dendrons described herein for generations 1-4 is shown in Table 2. The number of diacyl groups, linker groups, and terminating groups can be calculated based on g.

TABLE 2
Formulation of Dendrimer or Dendron Groups Based on Generation (g)

| | g = 1 | g = 2 | g = 3 | g = 4 | |
|---|---|---|---|---|---|
| # of diacyl grp | 1 | 1 + 2 = 3 | 1 + 2 + 2² = 7 | 1 + 2 + 2² + 2³ = 15 | 1 + 2 + . . . + 2^{g-1} |
| # of linker grp | 0 | 1 | 1 + 2 | 1 + 2 + 2² | 1 + 2 + . . . + 2^{g-2} |
| # of terminating grp | 1 | 2 | 2² | 2³ | 2^{(g-1)} |

In some embodiments, the diacyl group independently comprises a structural formula

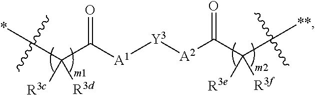

* indicates a point of attachment of the diacyl group at the proximal end thereof, and ** indicates a point of attachment of the diacyl group at the distal end thereof.

In some embodiments of the diacyl group of X_{Branch}, Y³ is independently at each occurrence an optionally substituted; alkylene, an optionally substituted alkenylene, or an optionally substituted arenylene. In some embodiments of the diacyl group of X_{Branch}, Y³ is independently at each occurrence an optionally substituted alkylene (e.g., C₁-C₁₂). In some embodiments of the diacyl group of X_{Branch}, Y³ is independently at each occurrence an optionally substituted alkenylene (e.g., C₁-C₁₂). In some embodiments of the diacyl group of X_{Branch}, Y³ is independently at each occurrence an optionally substituted arenylene (e.g., C₁-C₁₂).

In some embodiments of the diacyl group of X_{Branch}, A¹ and A² are each independently at each occurrence —O—, —S—, or —NR⁴—. In some embodiments of the diacyl group of X_{Branch}, A¹ and A² are each independently at each occurrence —O—. In some embodiments of the diacyl group of X_{Branch}, A¹ and A² are each independently at each occurrence —S—. In some embodiments of the diacyl group of X_{Branch}, A¹ and A² are each independently at each occurrence —NR⁴— and R⁴ is hydrogen or optionally substituted alkyl (e.g., C₁-C₆). In some embodiments of the diacyl group of X_{Branch}, m¹ and m² are each independently at each occurrence 1, 2, or 3. In some embodiments of the diacyl group of X_{Branch}, m¹ and m² are each independently at each occurrence 1. In some embodiments of the diacyl group of X_{Branch}, m¹ and m² are each independently at each occurrence 2. In some embodiments of the diacyl group of X_{Branch}, m¹ and m² are each independently at each occurrence 3. In some embodiments of the diacyl group of X_{Branch}, R^{3c}, R^{3d}, R^{3e}, and R^{3f} are each independently at each occurrence hydrogen or an optionally substituted alkyl. In some embodiments of the diacyl group of X_{Branch}, R^{3c}, R^{3d}, R^{3e}, and R^{3f} are each independently at each occurrence hydrogen. In some embodiments of the diacyl group of X_{Branch}, R^{3c}, R^{3d}, R^{3e}, and R^{3f} are each independently at each occurrence an optionally substituted (e.g., C₁-C₈) alkyl.

In some embodiments of the diacyl group, A¹ is —O— or —NH—. In some embodiments of the diacyl group, A¹ is —O—. In some embodiments of the diacyl group, A² is —O— or —NH—. In some embodiments of the diacyl group, A² is —O—. In some embodiments of the diacyl group, Y³ is C₁-C₁₂ (e.g., C₁-C₆, such as C₁-C₃) alkylene.

In some embodiments of the diacyl group, the diacyl group independently at each occurrence comprises a structural formula

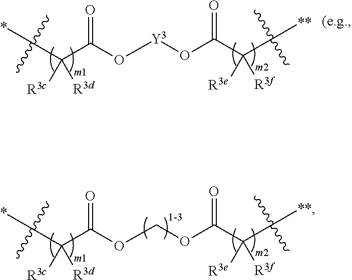

such as

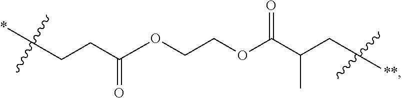

and optionally R^{3c}, R^{3d}, R^{3e}, and R^{3f} are each independently at each occurrence hydrogen or C₁-C₃ alkyl.

In some embodiments, linker group independently comprises a structural formula

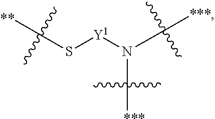

** indicates a point of attachment of the linker to a proximal diacyl group, and *** indicates a point of attachment of the linker to a distal diacyl group.

In some embodiments of the linker group of X_{Branch} if present, Y₁ is independently at each occurrence an optionally substituted alkylene, an optionally substituted alkenylene, or an optionally substituted arenylene. In some embodiments of the linker group of X_{Branch} if present, Y₁ is independently at each occurrence an optionally substituted alkylene (e.g., C₁-C₁₂). In some embodiments of the linker group of X_{Branch} if present, Y₁ is independently at each occurrence an optionally substituted alkenylene (e.g., C₁-C₁₂). In some embodiments of the linker group of X_{Branch} if present, Y₁ is independently at each occurrence an optionally substituted arenylene (e.g., C₁-C₁₂).

In some embodiments of the terminating group of X_{Branch}, each terminating group is independently selected from optionally substituted alkylthiol and optionally substituted alkenylthiol. In some embodiments of the terminating group of X_{Branch}, each terminating group is an optionally substituted alkylthiol (e.g., C₁-C₁₈, such as C₄-C₁₈). In some embodiments of the terminating group of X_{Branch}, each terminating group is optionally substituted alkenylthiol (e.g., C₁-C₁₈, such as C₄-C₁₈).

In some embodiments of the terminating group of X_{Branch}, each terminating group is independently C₁-C₁₈ alkenylthiol or C₁-C₁₈ alkylthiol, and the alkyl or alkenyl moiety is optionally substituted with one or more substituents each independently selected from halogen, C₆-C₁₂ aryl, C₁-C₁₂ alkylamino, C₄-C₆ N-heterocycloalkyl, —OH, —C(O)OH, —C(O)N(C₁-C₃ alkyl)-(C₁-C₆ alkylene)-(C₁-C₁₂ alkylamino), —C(O)N(C₁-C₃ alkyl)-(C₁-C₆ alkylene)-(C₄-C₆ N-heterocycloalkyl), —C(O)—(C₁-C₁₂ alkylamino), and —C(O)—(C₄-C₆ N-heterocycloalkyl), and the C₄-C₆ N-heterocycloalkyl moiety of any of the preceding substituents is optionally substituted with C₁-C₃ alkyl or C₁-C₃ hydroxyalkyl.

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

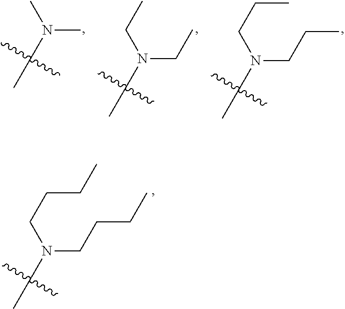

C₄-C₆ N-heterocycloalkyl (e.g., N-pyrrolidinyl

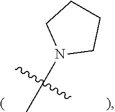 [SMILES: *N1CCCC1]

### N-piperidinyl

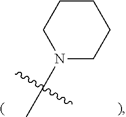 [SMILES: *N1CCCCC1]

### N-azepanyl

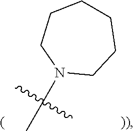 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino)) (e.g.,

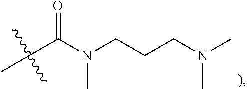 [SMILES: *C(=O)N(C)CCCN(C)C]

—C(O)N(C₁-C₃ alkyl)-(C₁-C₆ alkylene)-(C₄-C₆ N-heterocycloalkyl) (e.g.,

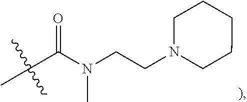 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl) (e.g.,

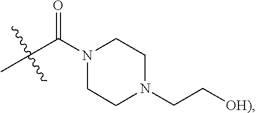

wherein the C₄-C₆ N-heterocycloalkyl moiety of any of the preceding substituents is optionally substituted with C₁-C₃ alkyl or C₁-C₃ hydroxyalkyl. In some embodiments of the terminating group of X_{Branch}, each terminating group is independently C₁-C₁₈ (e.g., C₄-C₁₈) alkylthiol, wherein the alkyl moiety is optionally substituted with one substituent —OH. In some embodiments of the terminating group of X_{Branch}, each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

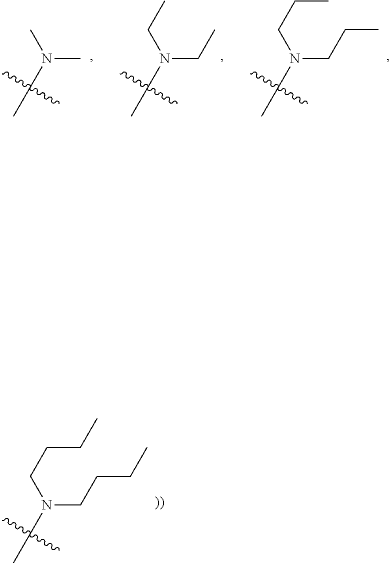

and C₄-C₆ N-heterocycloalkyl (e.g., N-pyrrolidinyl

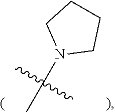 [SMILES: *N1CCCC1]

### N-piperidinyl

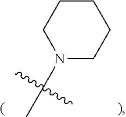 [SMILES: *N1CCCCC1]

### N-azepanyl

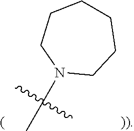 [SMILES: *N1CCCCCC1]

In some embodiments of the terminating group of X_{Branch}, each terminating group is independently C₁-C₁₈ (e.g., C₄-C₁₈) alkenylthiol or C₁-C₁₈ (e.g., C₄-C₁₈) alkylthiol. In some embodiments of the terminating group of X_{Branch}, each terminating group is independently C₁-C₁₈ (e.g., C₄-C₁₈) alkylthiol.

In some embodiments of the terminating group of X_{Branch}, each terminating group is independently a structural set forth in Table 3. In some embodiments, the dendrimers or dendrons described herein can comprise a terminating group or pharmaceutically acceptable salt, or thereof selected in Table 3. In some embodiments, the example terminating group of Table 3 are not limiting of the stereoisomers (i.e., enantiomers, diastereomers) listed.

TABLE 3
Example terminating group/peripheries structures

| ID # | Structure |
|---|---|
| SC1 | 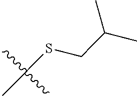 [SMILES: *SCC(C)C] |
| SC2 | 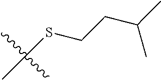 [SMILES: *SCCC(C)C] |
| SC3 | 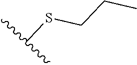 [SMILES: *SCCC] |
| SC4 | 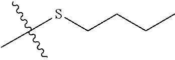 [SMILES: *SCCCC] |
| SC5 | 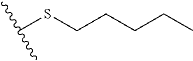 [SMILES: *SCCCCC] |
| SC6 | 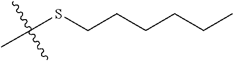 [SMILES: *SCCCCCC] |
| SC7 | 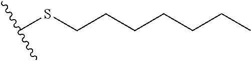 [SMILES: *SCCCCCCC] |
| SC8 | 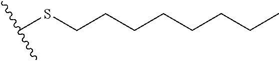 [SMILES: *SCCCCCCCC] |
| SC9 | 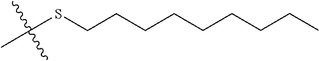 [SMILES: *SCCCCCCCCC] |
| SC10 | 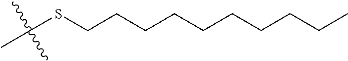 [SMILES: *SCCCCCCCCCC] |
| SC11 | 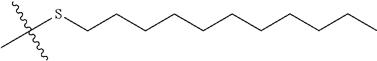 [SMILES: *SCCCCCCCCCCC] |
| SC12 | 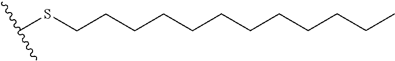 [SMILES: *SCCCCCCCCCCCC] |
| SC14 | 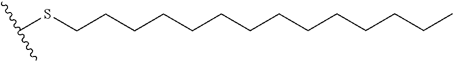 [SMILES: *SCCCCCCCCCCCCCC] |
| SC16 | 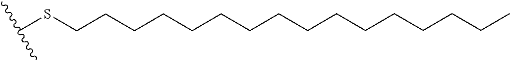 [SMILES: *SCCCCCCCCCCCCCCCC] |
| SC18 | 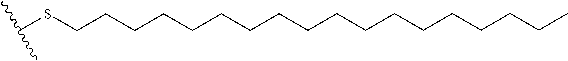 [SMILES: *SCCCCCCCCCCCCCCCCCC] |
| SC19 | 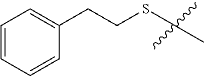 [SMILES: *SCCc1ccccc1] |
| SO1 | 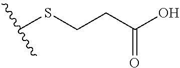 [SMILES: *SCCC(=O)O] |
| SO2 | 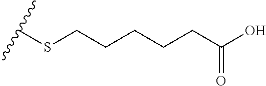 [SMILES: *SCCCCCC(=O)O] |
| SO3 | 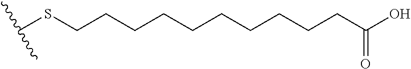 [SMILES: *SCCCCCCCCCCC(=O)O] |
| SO4 | 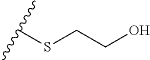 [SMILES: *SCCO] |
| SO5 | 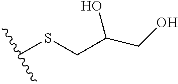 [SMILES: *SCC(O)CO] |
| SO6 | 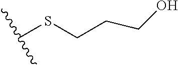 [SMILES: *SCCCO] |
| SO7 | 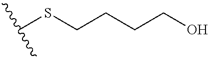 [SMILES: *SCCCCO] |
| SO8 | 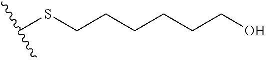 [SMILES: *SCCCCCCO] |
| SO9 | 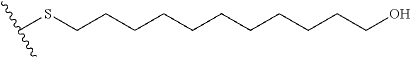 [SMILES: *SCCCCCCCCCCCO] |
| SN1 | 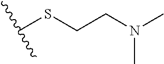 [SMILES: *SCCN(C)C] |
| SN2 | 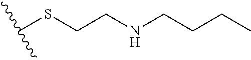 [SMILES: *SCCNCCCC] |
| SN3 | 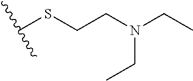 [SMILES: *SCCN(CC)CC] |
| SN4 | 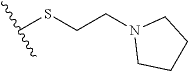 [SMILES: *SCCN1CCCC1] |
| SN5 | 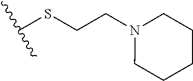 [SMILES: *SCCN1CCCCC1] |
| SN6 | 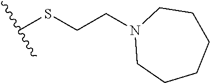 [SMILES: *SCCN1CCCCCC1] |
| SN7 | 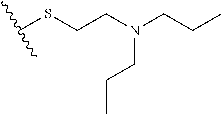 [SMILES: *SCCN(CCC)CCC] |
| SN8 | 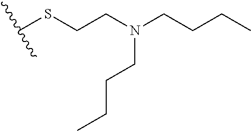 [SMILES: *SCCN(CCCC)CCCC] |
| SN9 | 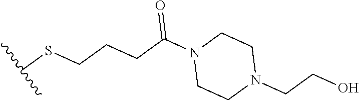 [SMILES: *SCCCC(=O)N1CCN(CCO)CC1] |
| SN10 | 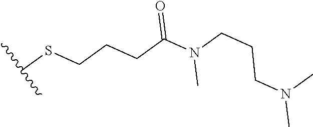 [SMILES: *SCCCC(=O)N(C)CCCN(C)C] |
| SN11 | 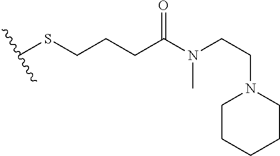 [SMILES: *SCCCC(=O)N(C)CCN1CCCCC1] |

In some embodiments, the dendrimer or dendron of Formula (X) is selected from those set forth in Table 4 and pharmaceutically acceptable salts thereof.

TABLE 4
Example ionizable cationic lipo-dendrimers or lipo-dendrons

| ID # | Structure |
|---|---|
| 2A2-SC14 | 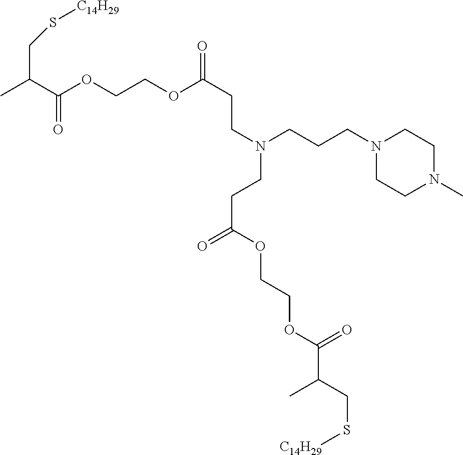 |
| 2A6-SC14 | 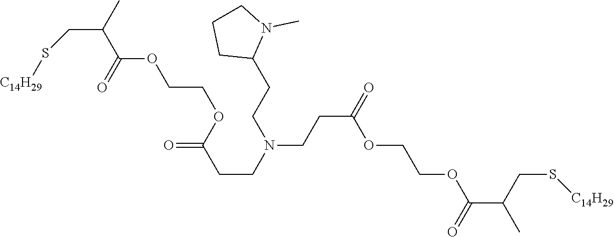 |
| 2A9-SC14 | 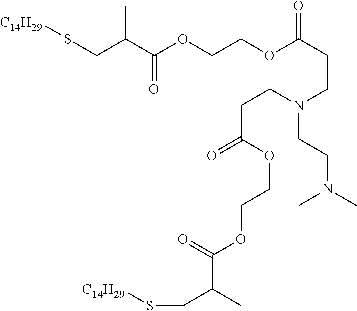 |
| 3A3-SC10 | 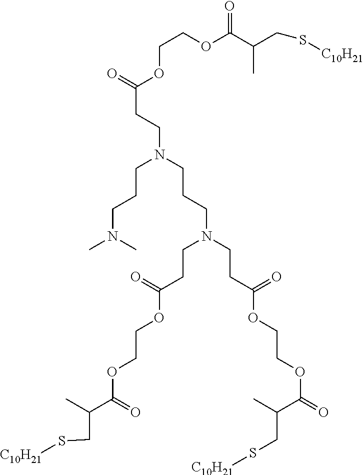 |
| 3A3-SC14 | 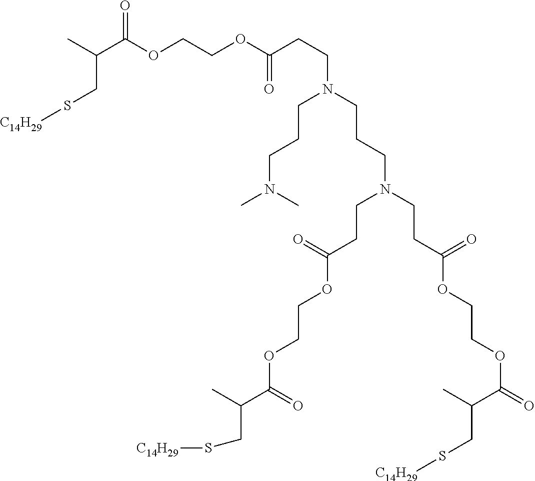 |
| 3A5-SC10 | 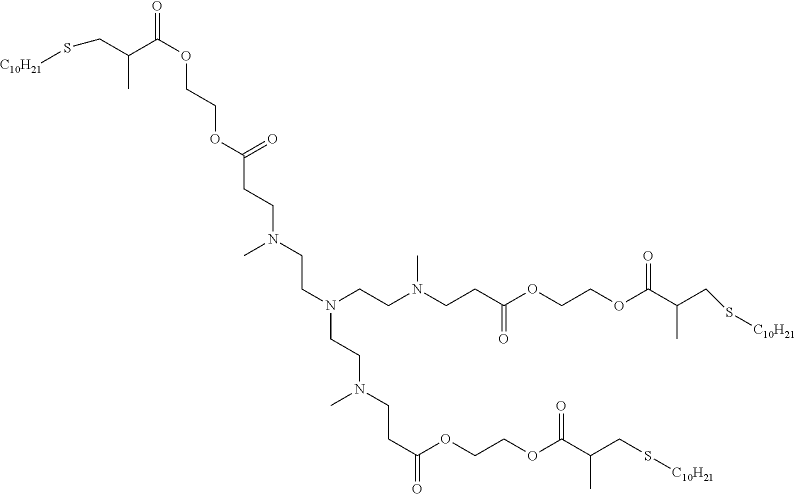 |
| 3A5-SC14 | 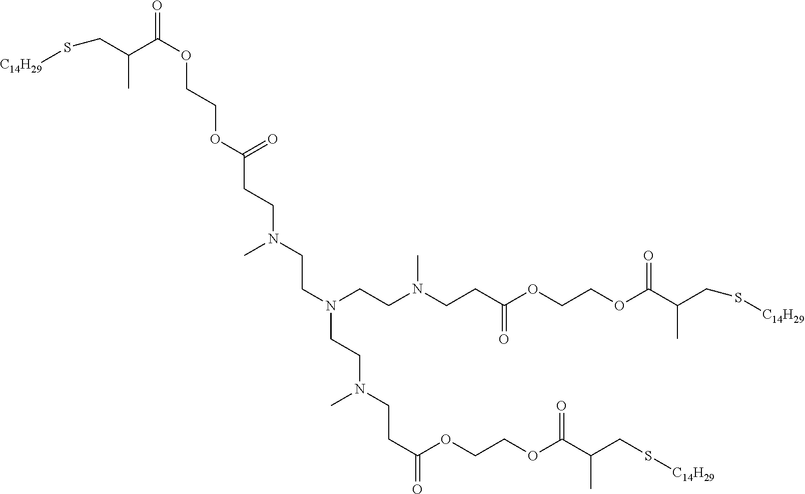 |
| 4A1-SC12 | 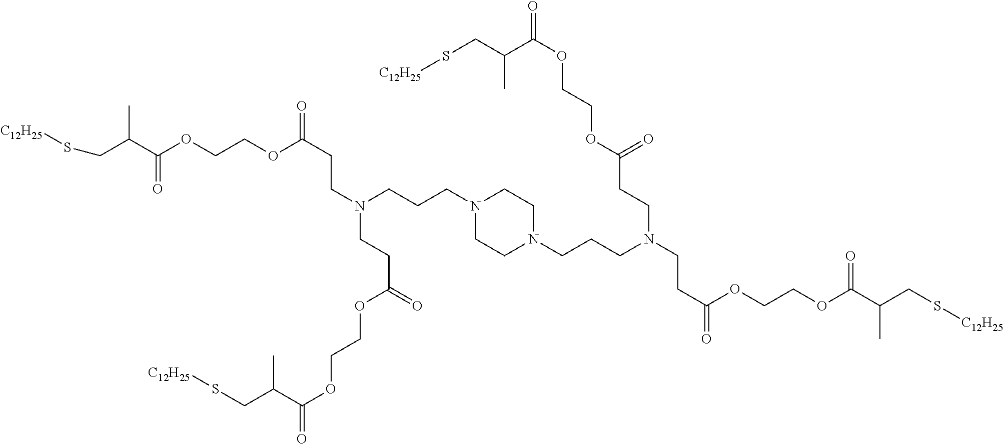 |
| 4A3-SC12 | 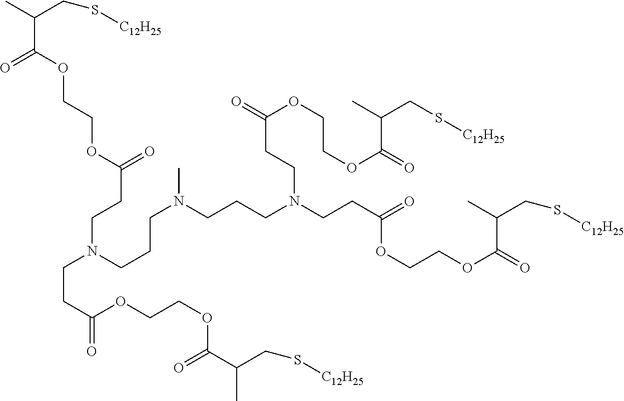 |
| 5A1-SC12 | 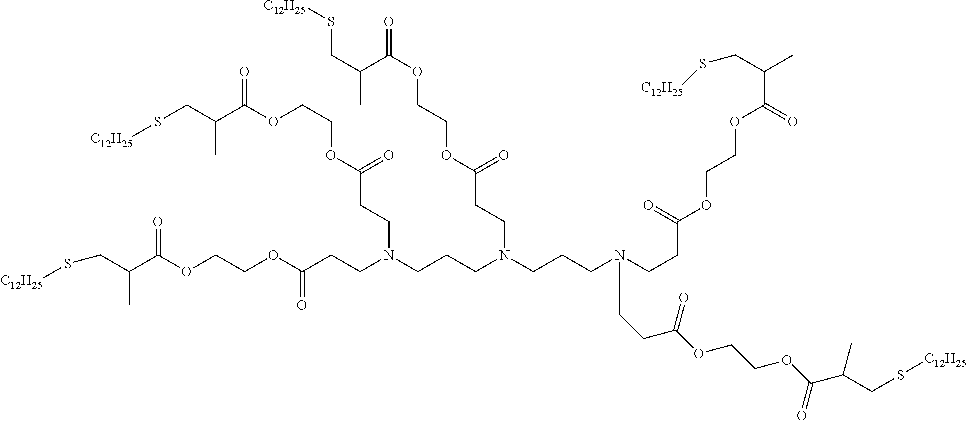 |
| 5A1-SC8 | 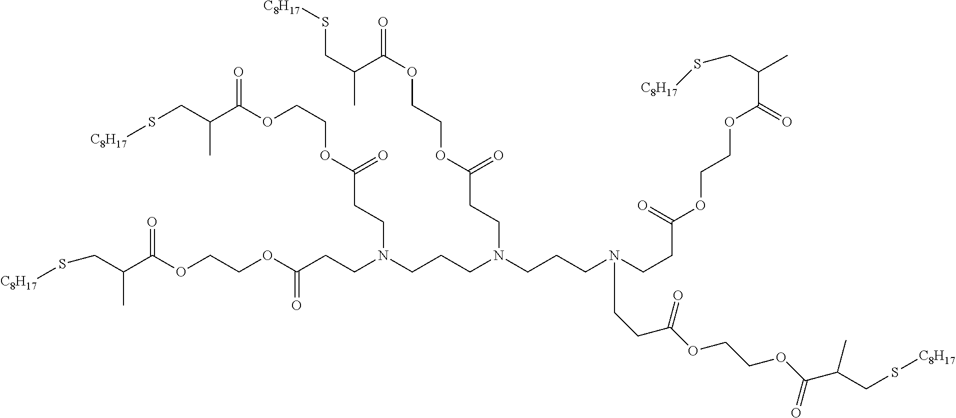 |
| 5A2-2-SC12 (5-arm) | 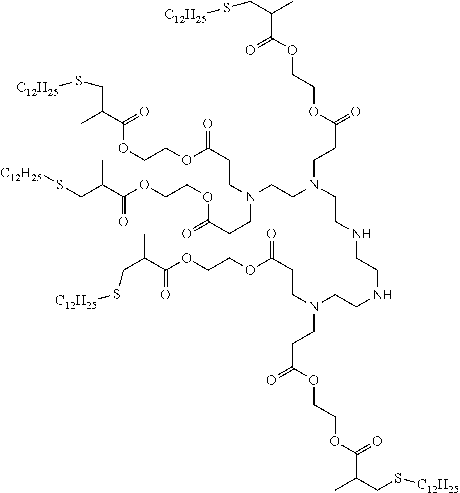 |
| 5A3-1-SC12 (5 arm) | 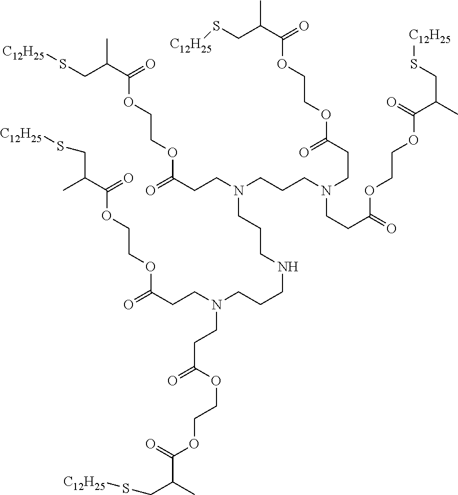 |
| 5A3-1-SC8 (5-arm) | 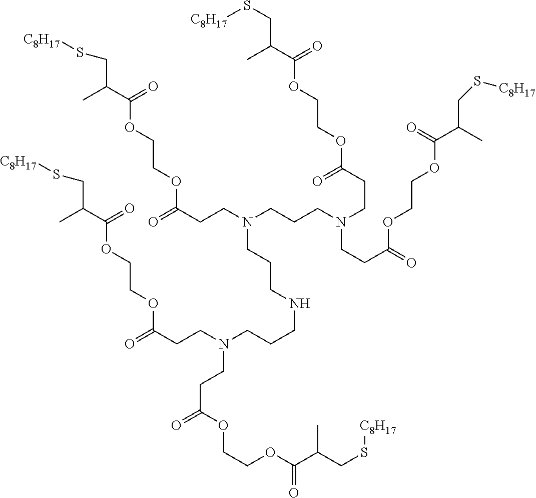 |
| 5A4-1-SC12 (5-arm) | 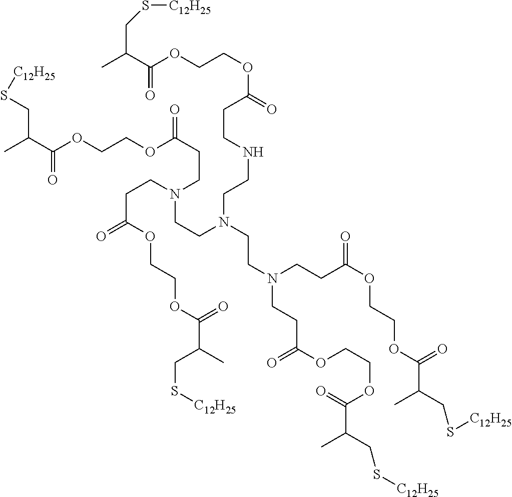 |
| 5A4-1-SC8 (5-arm) | 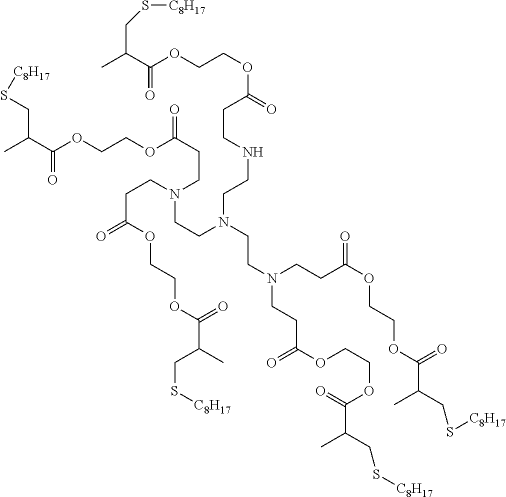 |
| 5A5-SC8 | 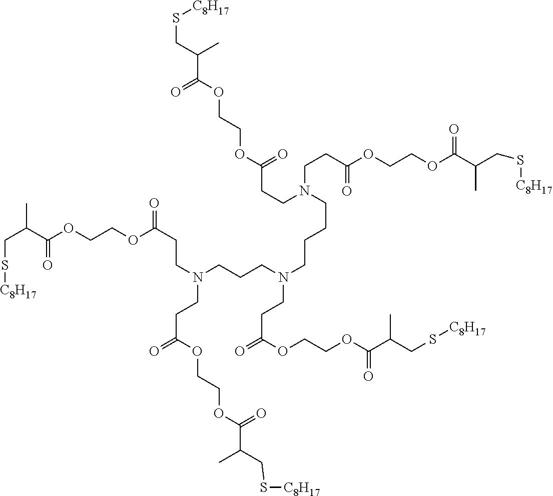 |
| 5A5-SC12 | 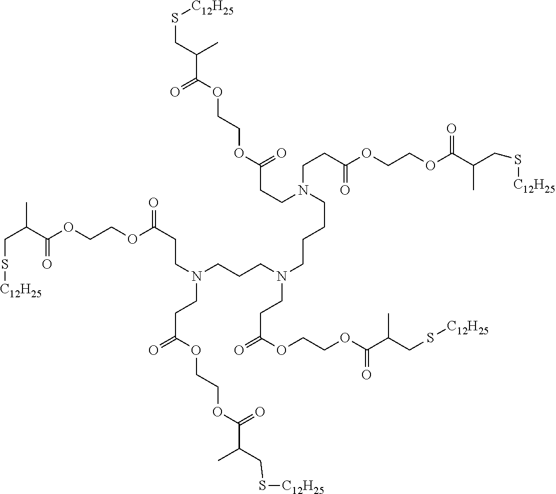 |
| 5A2-4-SC12 (6-arm) | 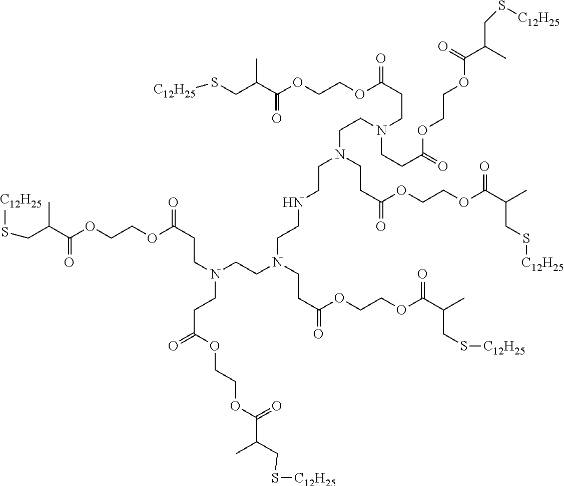 |
| 5A2-4-SC10 (6-arm) | 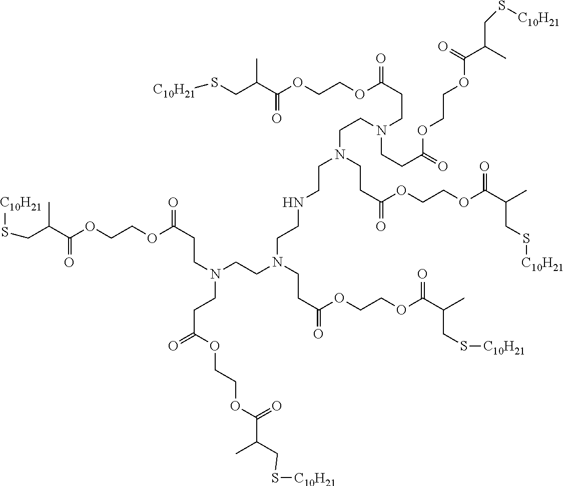 |
| 5A3-2-SC8 (6-arm) | 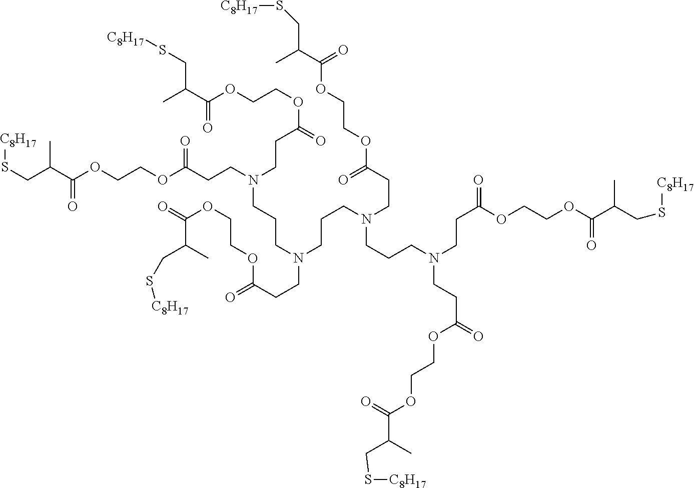 |
| 5A3-2-SC12 (6-arm) | 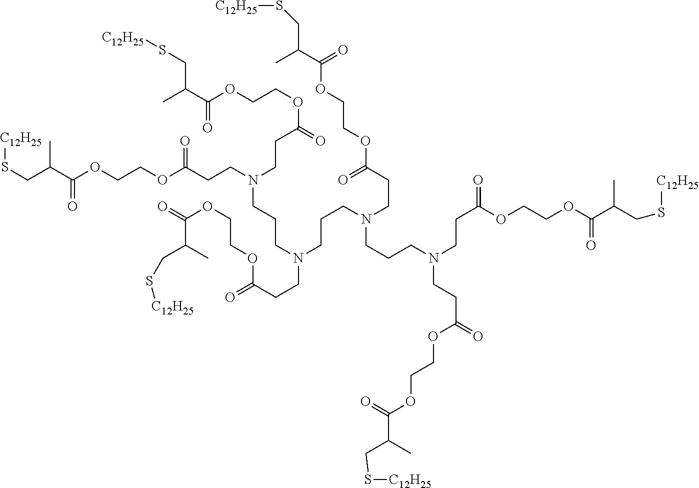 |
| 5A4-2-SC8 (6-arm) | 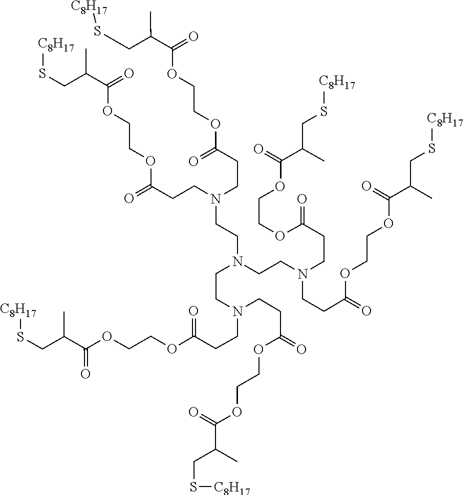 |
| 5A4-2-SC12 (6-arm) | 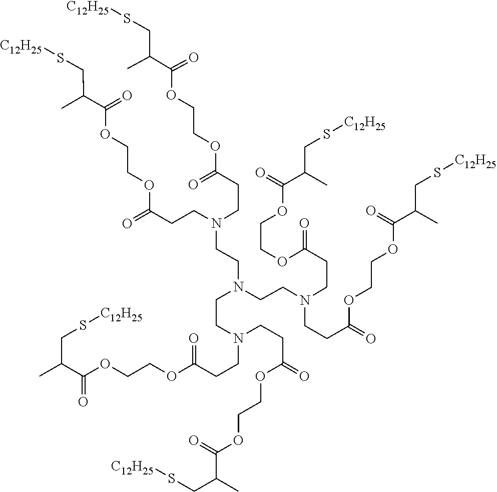 |
| 6A4-SC8 | 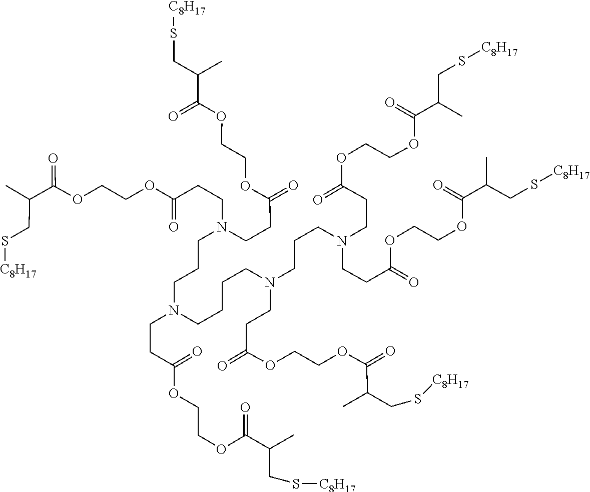 |
| 6A4-SC12 | 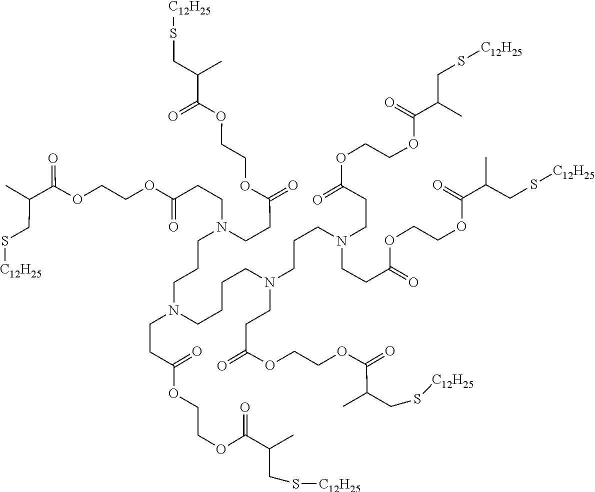 |
| 2A2-g2-SC12 | 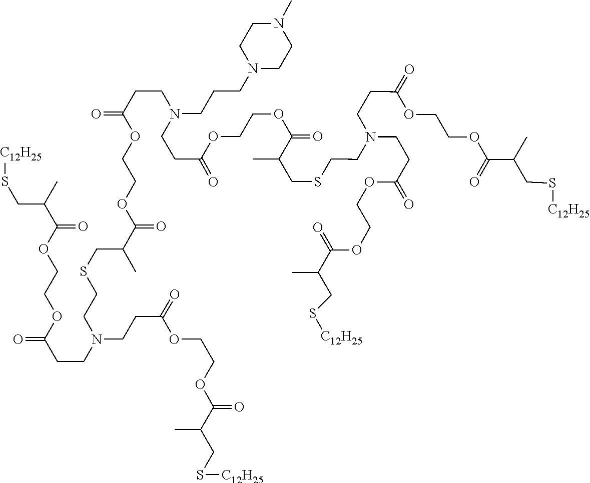 |
| 2A2-g2-SC8 | 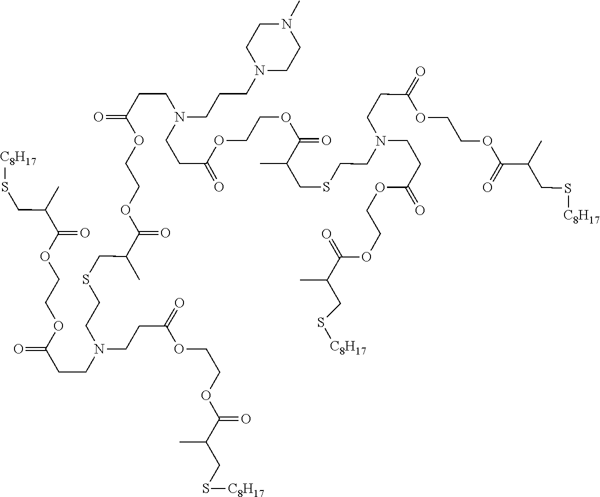 |
| 2A11-g2-SC12 | 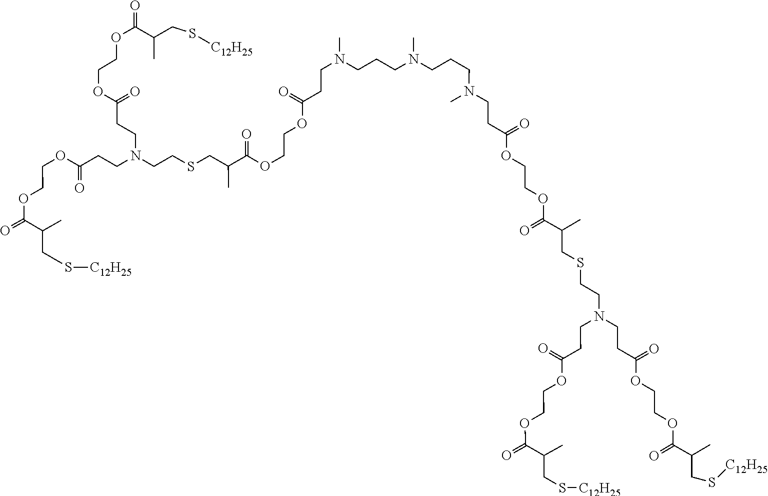 |
| 2A11-g2-SC8 | 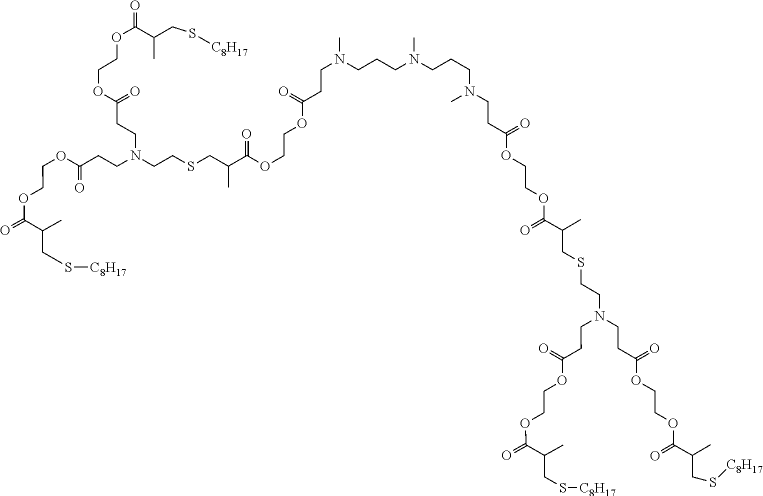 |
| 3A3-g2-SC12 | 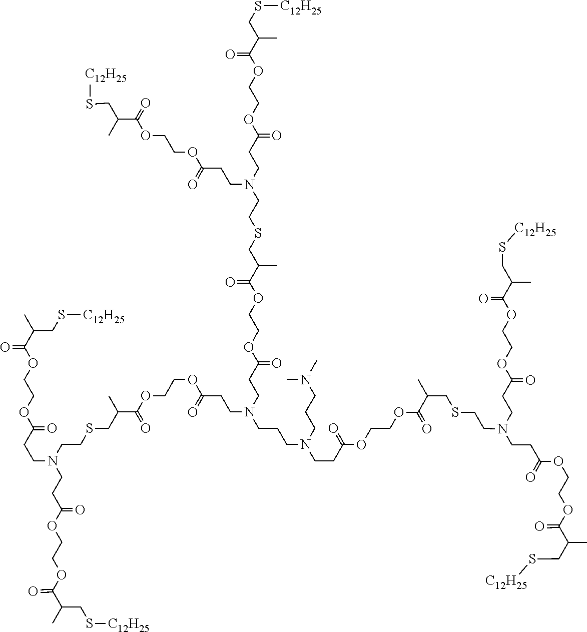 |
| 3A3-g2-SC8 | 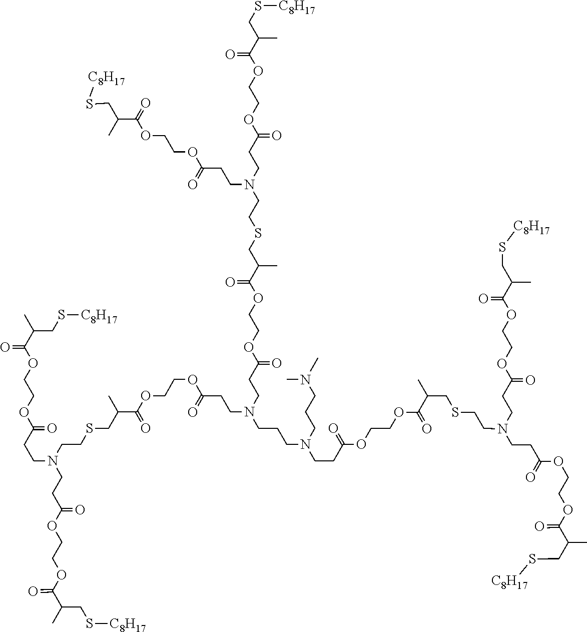 |
| 3A5-g2-SC12 | 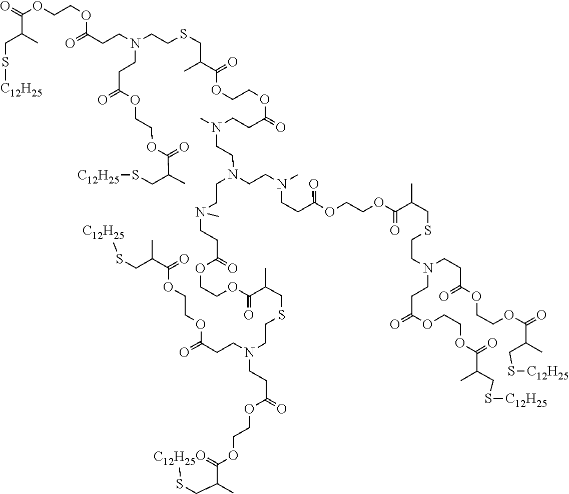 |
| 2A11-g3-SC12 | 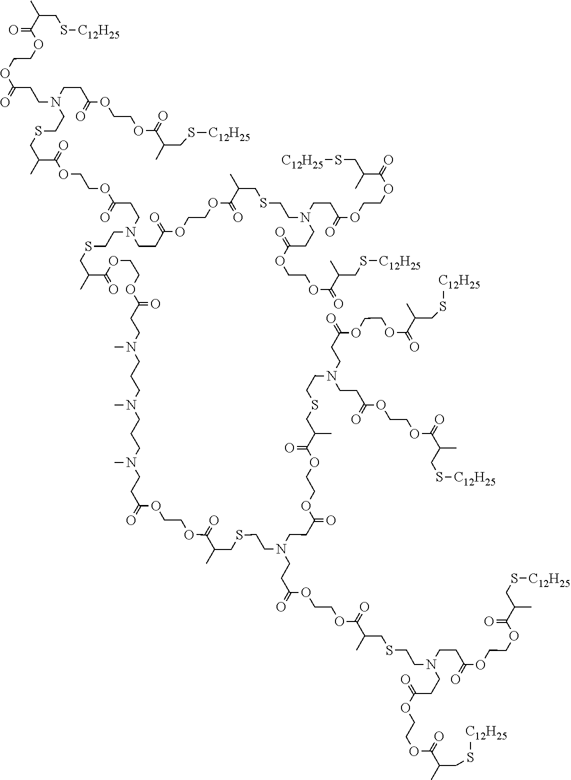 |
| 2A11-g3-SC8 | 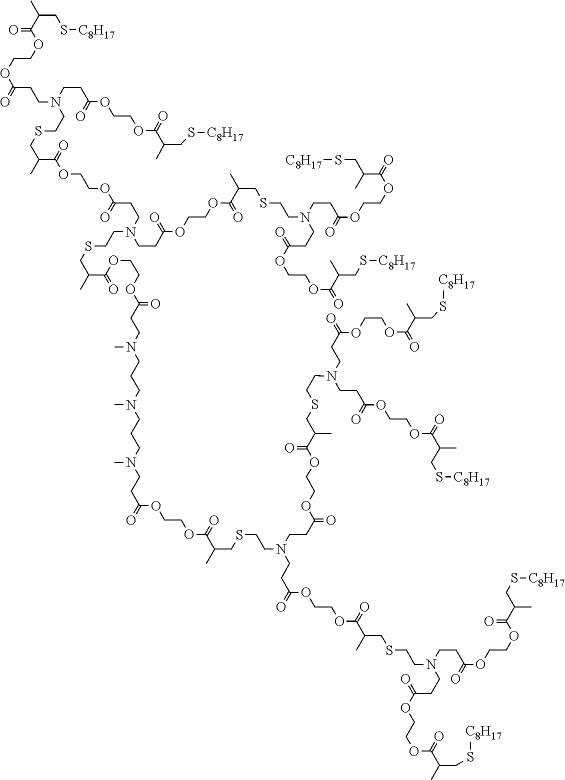 |
| 1A2-g4-SC12 | 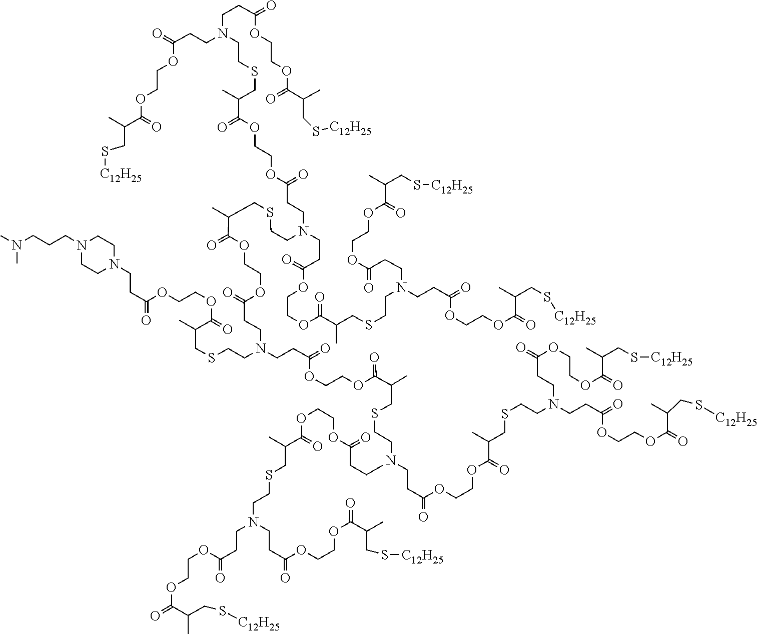 |
| 4A1-g2-SC12 | 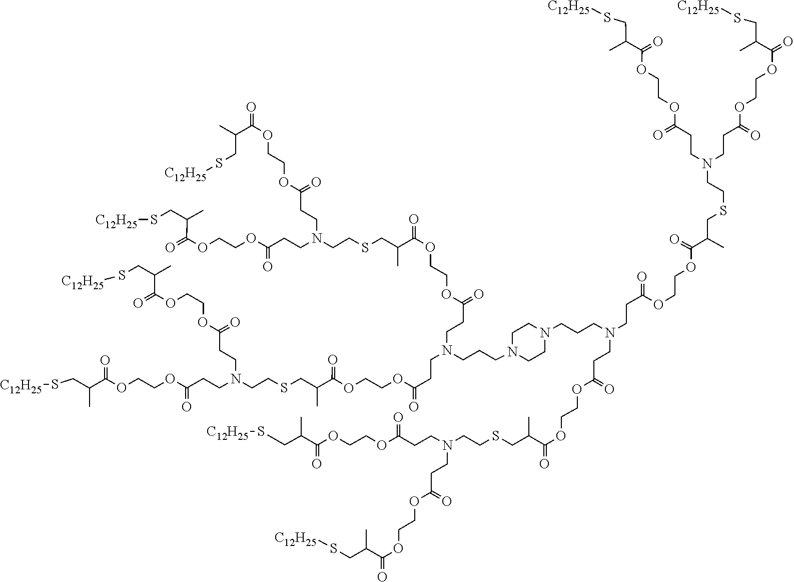 |
| 1A2-g4-SC8 | 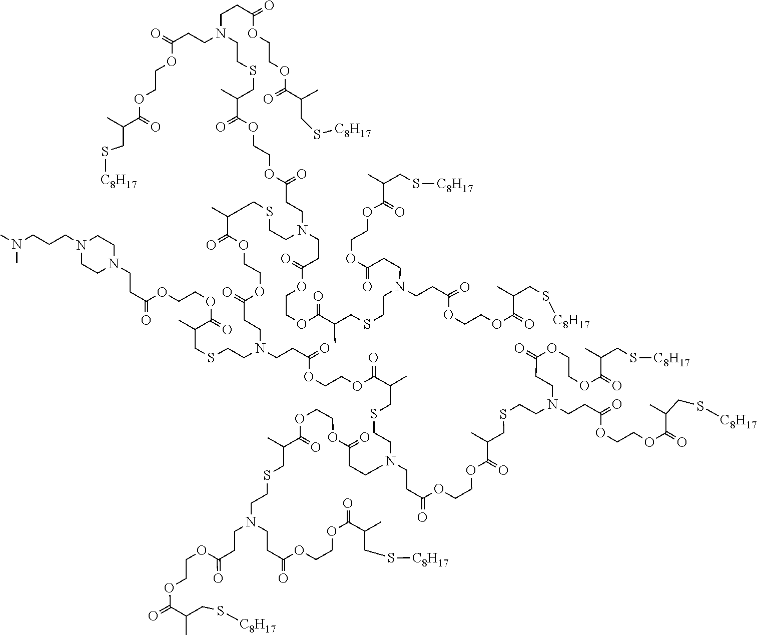 |
| 4A1-g2-SC8 | 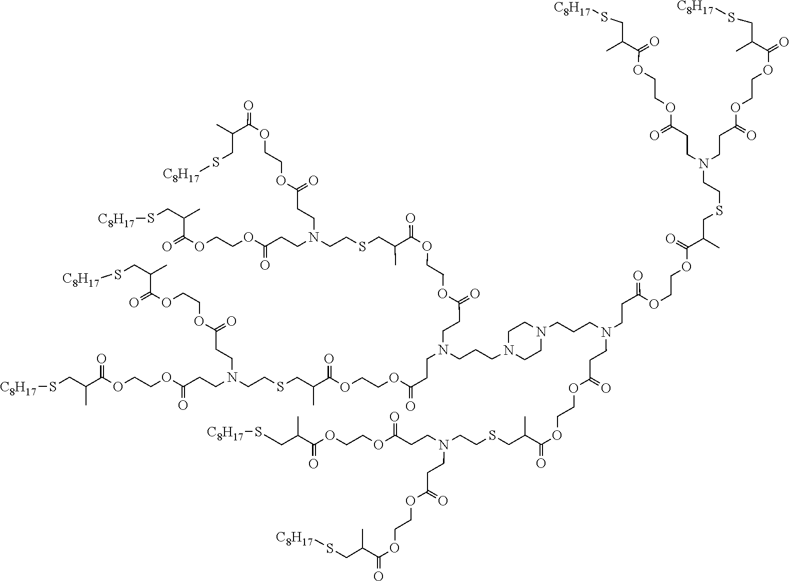 |
| 4A3-g2-SC12 | 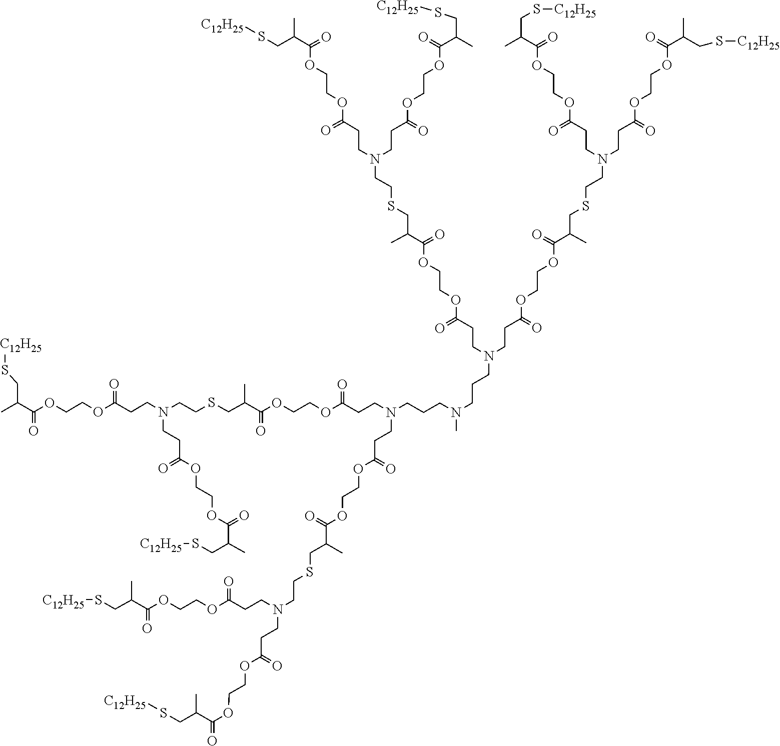 |
| 4A3-g2-SC8 | 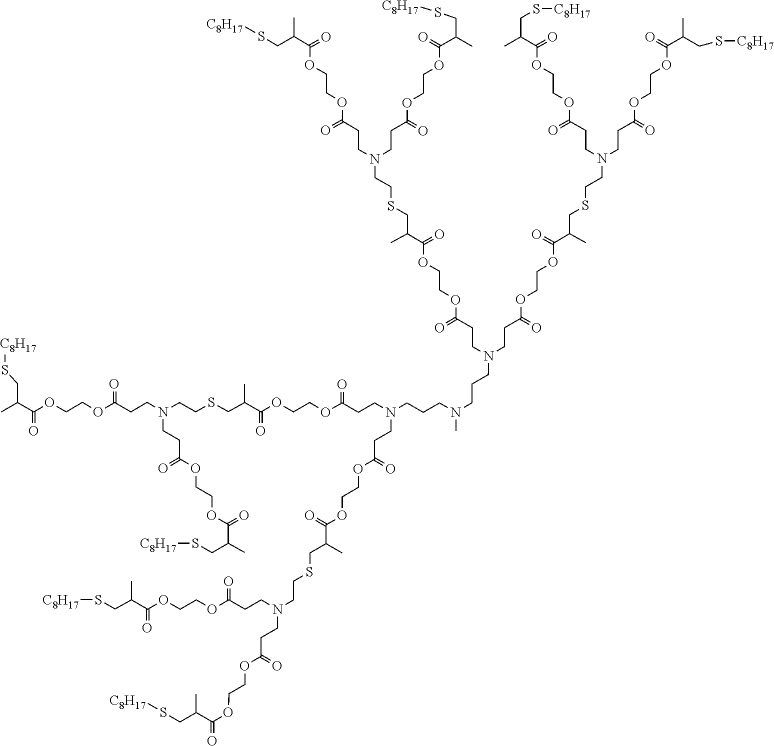 |
| 1A2-g3-SC12 | 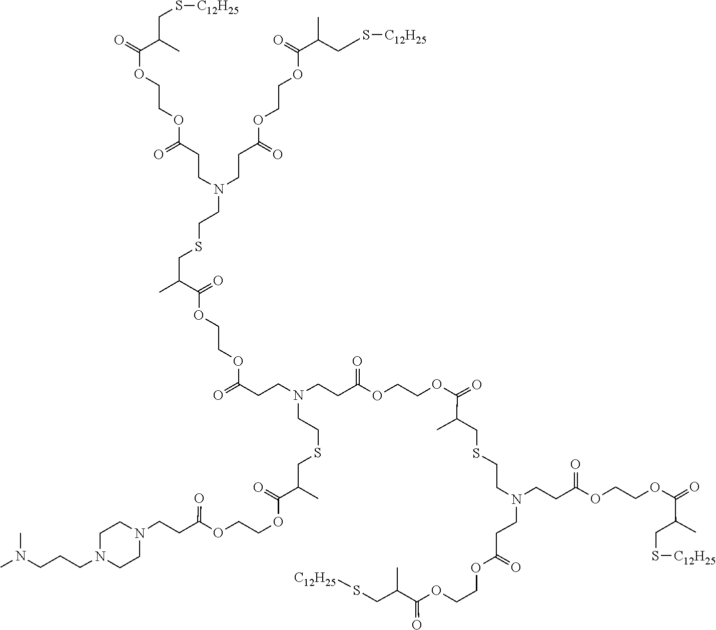 |
| 1A2-g3-SC8 | 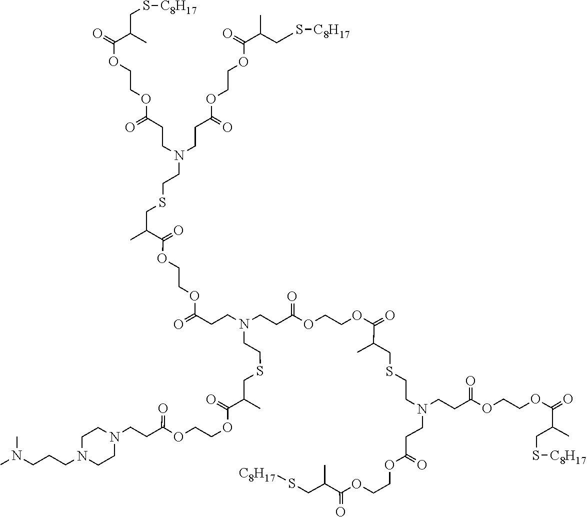 |
| 2A2-g3-SC12 | 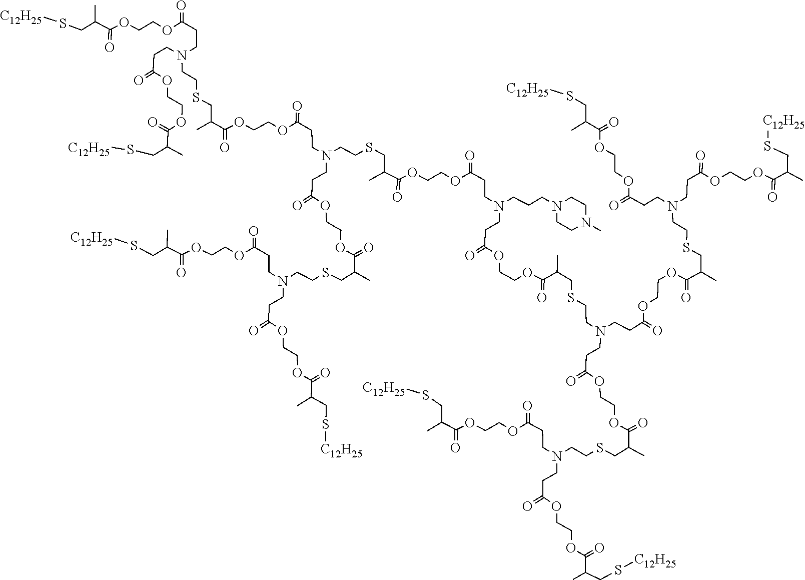 |
| 2A2-g3-SC8 | 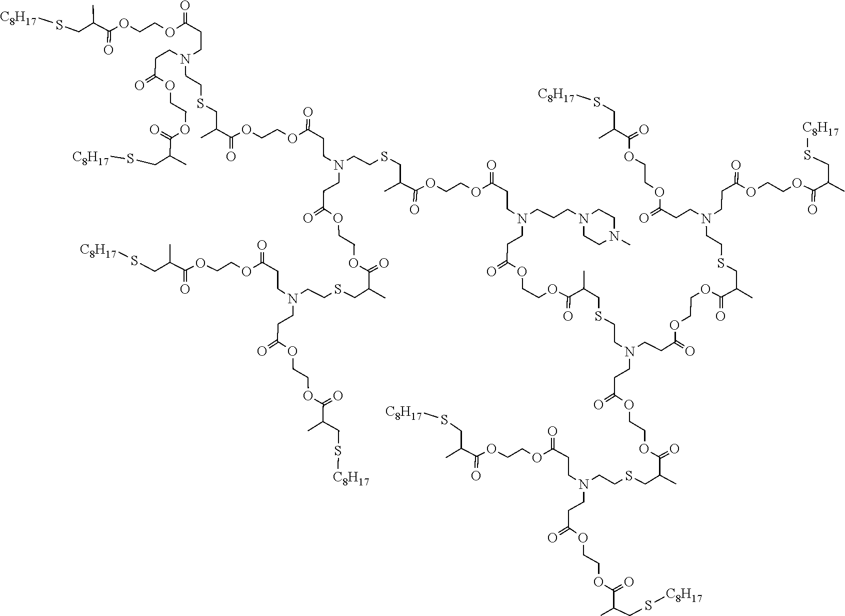 |
| 5A2-4-SC8 (6-arm) | 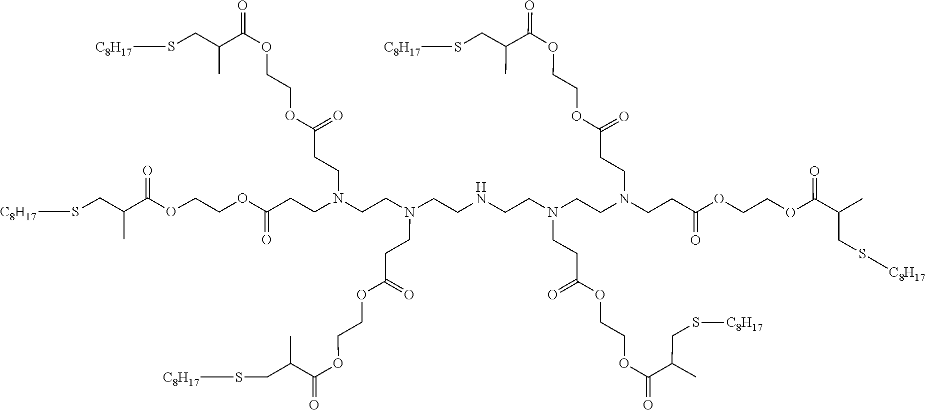 |
| 5A-5-SC8 (6 arm) | 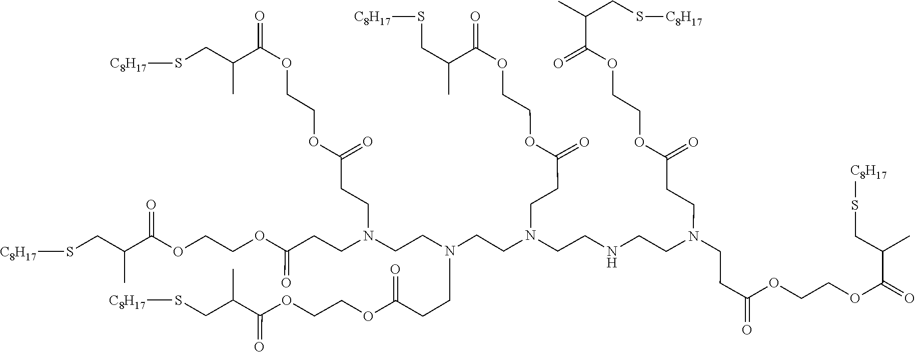 |
| 5A2-6-SC8 (6 arm) | 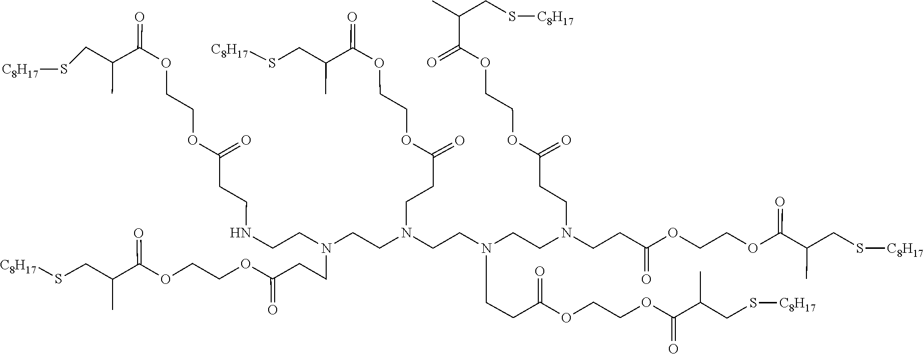 |
| 5A2-1-SC8 (5-arm) | 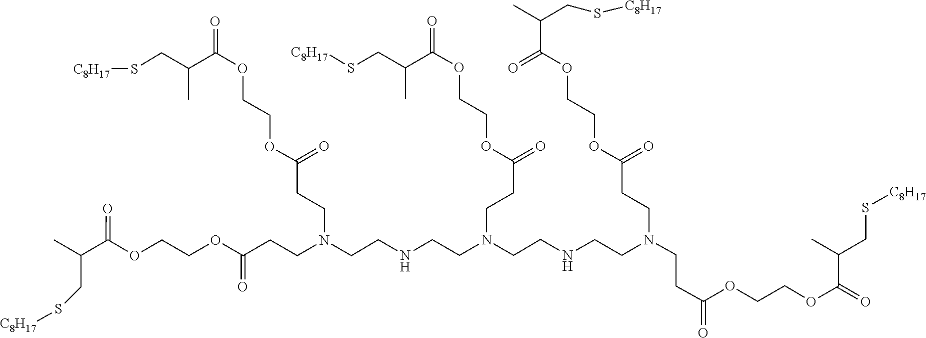 |
| 5A2-2-SC8 | 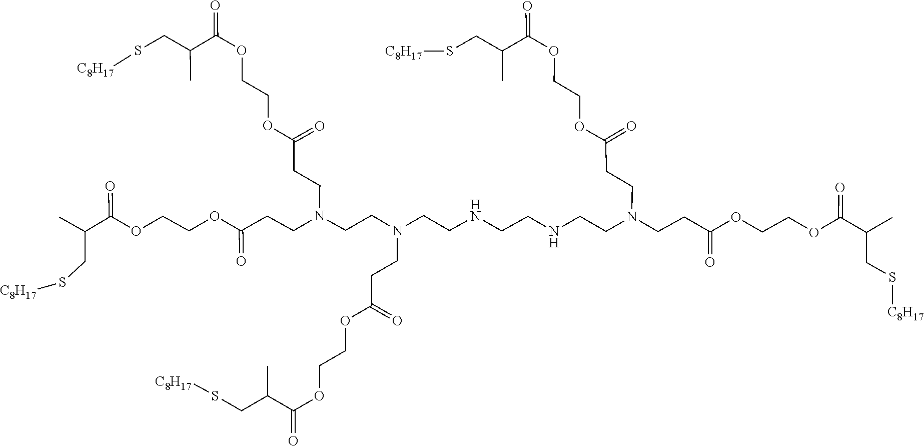 |
| 4A1-SC5 | 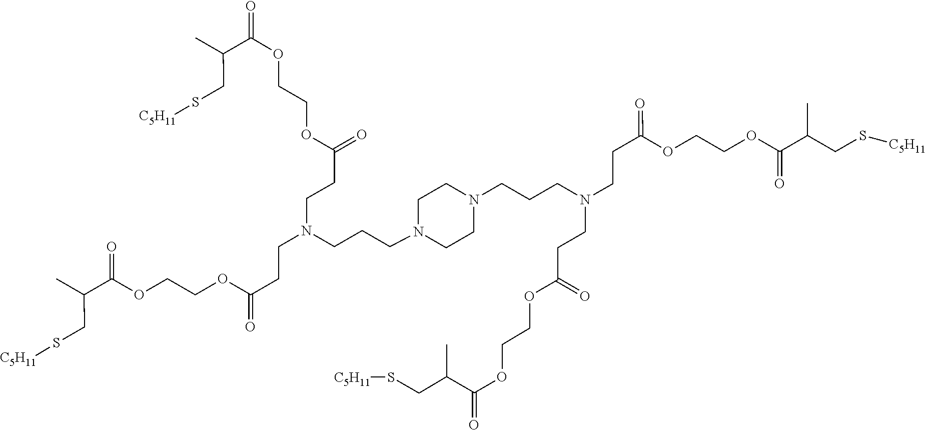 |
| 4A1-SC8 | 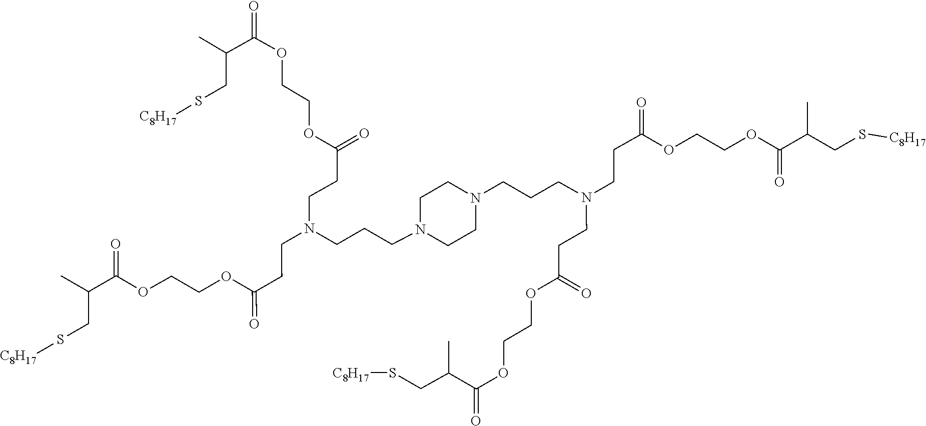 |
| 4A3-SC6 | 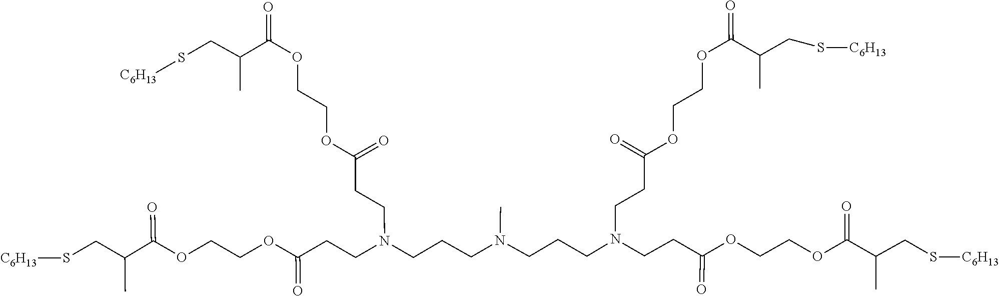 |
| 4A3-SC7 | 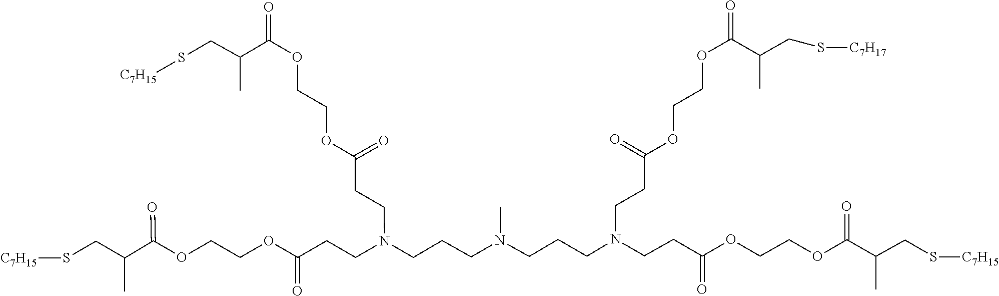 |
| 4A3-SC8 | 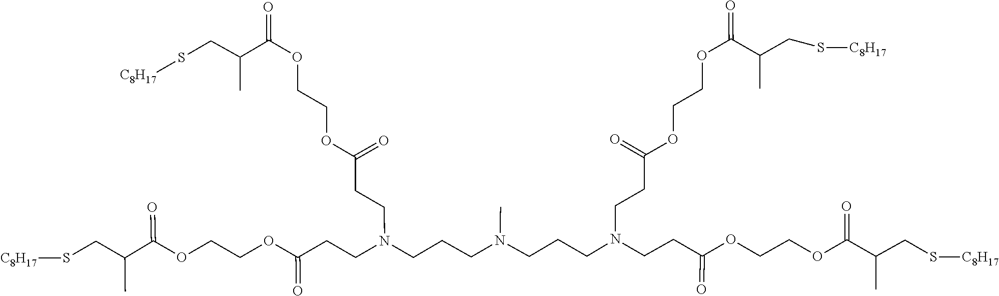 |
| 5A4-2-SC5 (6 arm) | 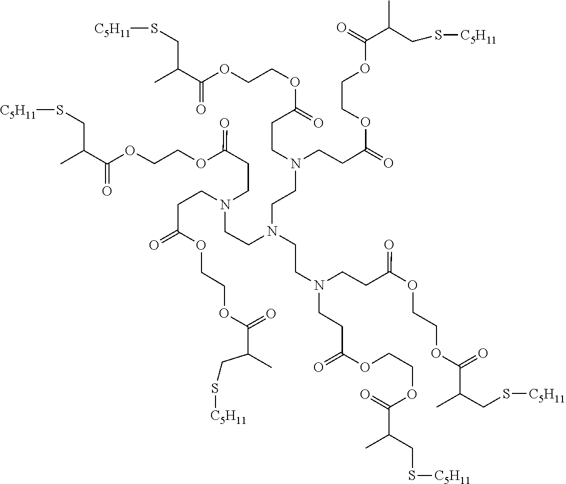 |
| 5A4-2-SC6 (6 arm) | 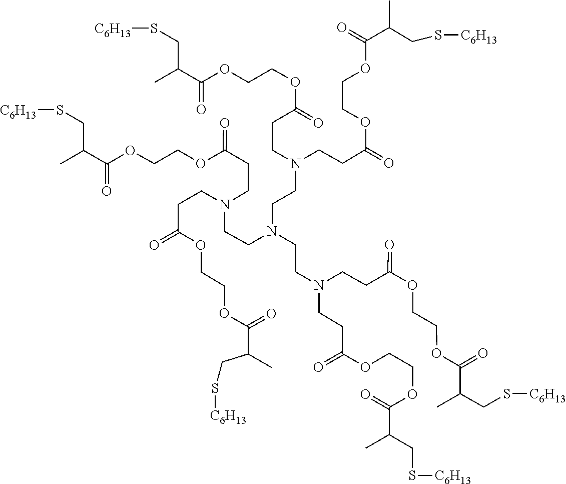 |
| 5A2-4-SC8 (5-arm) | 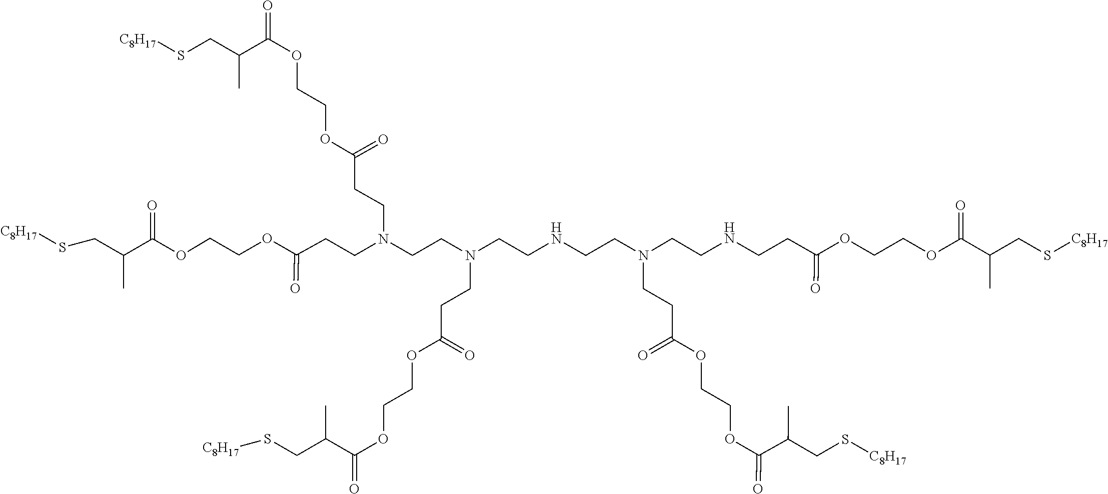 |
| 3A5-g2-SC8 | 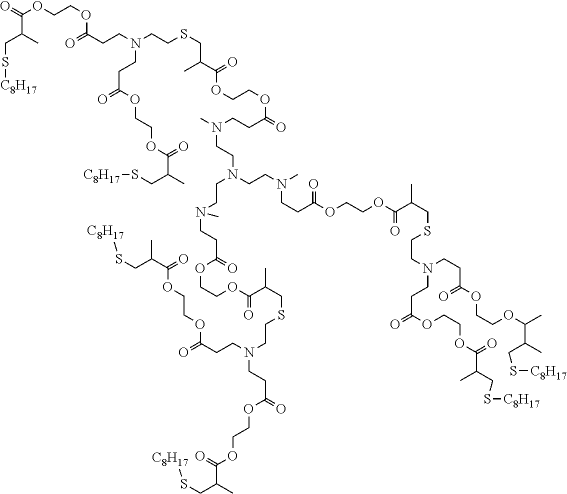 |

### Other Ionizable Cationic Lipids

In some embodiments of the lipid composition, the cationic lipid comprises a structural formula (D-I′):

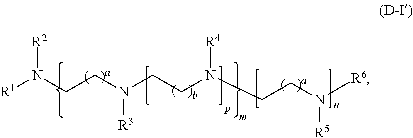

wherein:
a is 1 and b is 2, 3, or 4; or, alternatively, b is 1 and a is 2, 3, or 4;
m is 1 and n is 1; or, alternatively, m is 2 and n is 0; or, alternatively, m is 2 and n is 1; and
R¹, R², R³, R⁴, R⁵, and R⁶ are each independently selected from the group consisting of H, —CH₂CH(OH)R⁷, —CH(R⁷)CH₂OH, —CH₂CH₂C(═O)OR⁷, —CH₂CH₂C(═O)NHR⁷, and —CH₂R⁷, wherein R⁷ is independently selected from C₃-C₁₈ alkyl, C₃-C₁₈ alkenyl having one C═C double bond, a protecting group for an amino group, —C(═NH)NH₂, a poly(ethylene glycol) chain, and a receptor ligand;
provided that at least two moieties among R¹ to R⁶ are independently selected from —CH₂CH(OH)R⁷, —CH(R⁷)CH₂OH, —CH₂CH₂C(═O)OR⁷, —CH₂CH₂C(═O)NHR⁷, or —CH₂R⁷, wherein R⁷ is independently selected from C₃-C₁₈ alkyl or C₃-C₁₈ alkenyl having one C═C double bond; and
wherein one or more of the nitrogen atoms indicated in formula (D-I′) may be protonated to provide a cationic lipid.

In some embodiments of the cationic lipid of formula (D-I′), a is 1. In some embodiments of the cationic lipid of formula (D-I′), b is 2. In some embodiments of the cationic lipid of formula (D-I′), m is 1. In some embodiments of the cationic lipid of formula (D-I′), n is 1. In some embodiments of the cationic lipid of formula (D-I′), R¹, R², R³, R⁴, R, and R⁶ are each independently H or —CH₂CH(OH)R⁷. In some embodiments of the cationic lipid of formula (D-I′), R¹, R², R³, R⁴, R⁵, and R⁶ are each independently H or

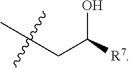

In some embodiments of the cationic lipid of formula (D-I′), R¹, R², R³, R⁴, R⁵, and R⁶ are each independently H or

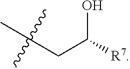

In some embodiments of the cationic lipid of formula (D-I′), R⁷ is C₃-C₁₈ alkyl (e.g., C₆-C₁₂ alkyl).

In some embodiments, the cationic lipid of formula (D-I′) is 13,16,20-tris(2-hydroxydodecyl)-13,16,20,23-tetraazapentatricontane-11,25-diol:

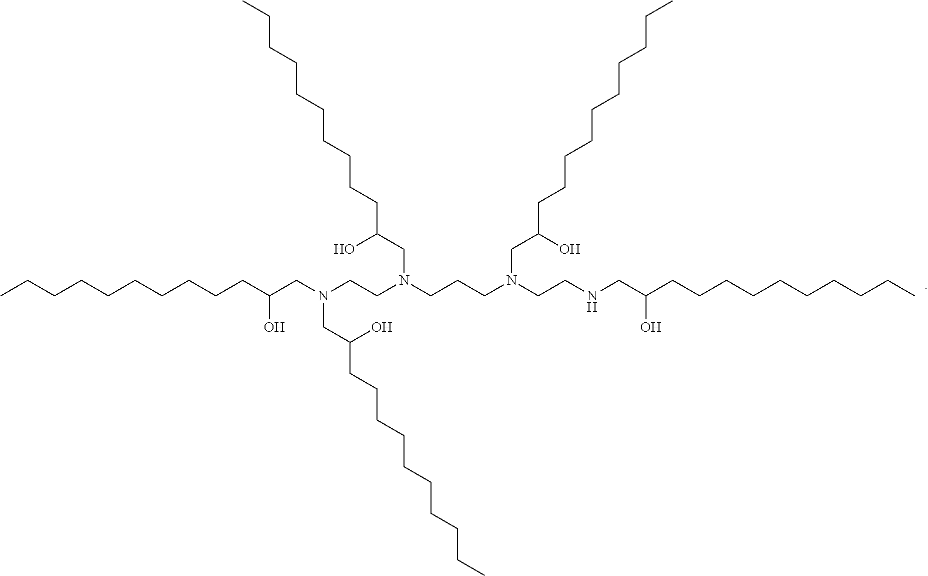 [SMILES: CCCCCCCCCCC(O)CNCCN(CCCN(CCN(CC(O)CCCCCCCCCC)CC(O)CCCCCCCCCC)CC(O)CCCCCCCCCC)CC(O)CCCCCCCCCC]

In some embodiments, the cationic lipid of formula (D-I′) is (11R,25R)-13,16,20-tris((R)-2-hydroxydodecyl)-13,16,20,23-tetraazapentatricontane-11,25-diol:

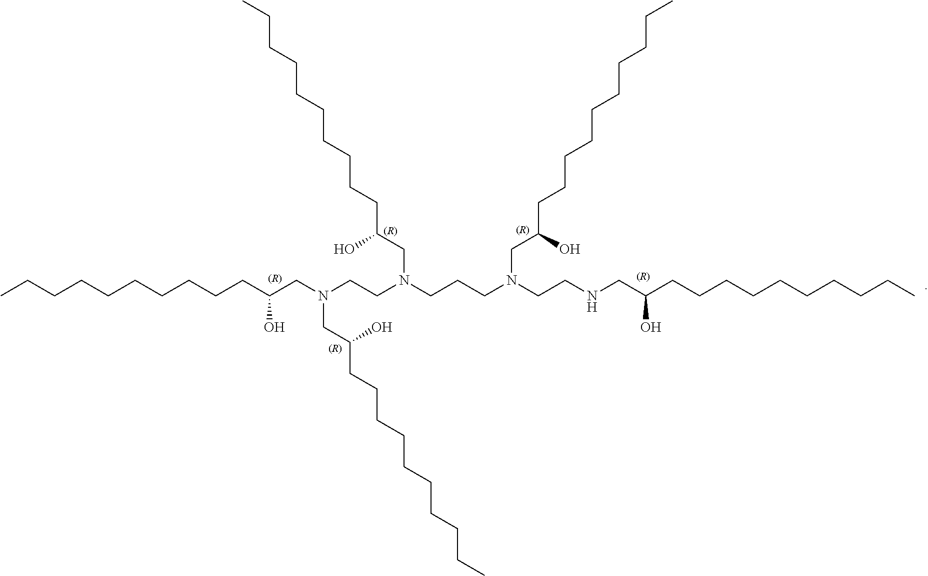

Additional cationic lipids that can be used in the compositions and methods of the present application include those cationic lipids as described in J. McClellan, M. C. King, Cell 2010, 141, 210-217, and International Patent Publication WO 2010/144740, WO 2013/149140, WO 2016/118725, WO 2016/118724, WO 2013/063468, WO 2016/205691, WO 2015/184256, WO 2016/004202, WO 2015/199952, WO 2017/004143, WO 2017/075531, WO 2017/117528, WO 2017/049245, WO 2017/173054 and WO 2015/095340, which are incorporated herein by reference for all purposes. Examples of those ionizable cationic lipids include but are not limited to those as shown in Table 5.

TABLE 5
Example ionizable cationic lipids

| # | Structure of example ionizable cationic lipid |
|---|---|
| 1 | 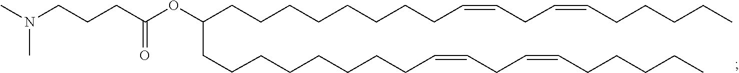 [SMILES: CCCCC/C=C\C/C=C\CCCCCCCCC(CCCCCCCC/C=C\C/C=C\CCCCC)OC(=O)CCCN(C)C] |
| 2 | 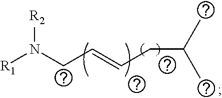 |
| 3 | 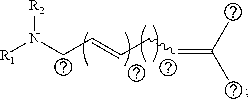 |
| 4 | 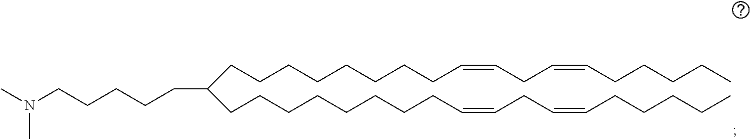 [SMILES: CCCCC/C=C\C/C=C\CCCCCCCCC(CCCCCCCC/C=C\C/C=C\CCCCC)CCCCCN(C)C] |
| 5 | 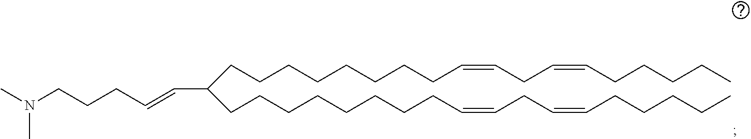 [SMILES: CCCCC/C=C\C/C=C\CCCCCCCCC(/C=C/CCCN(C)C)CCCCCCCC/C=C\C/C=C\CCCCC] |
| 6 | 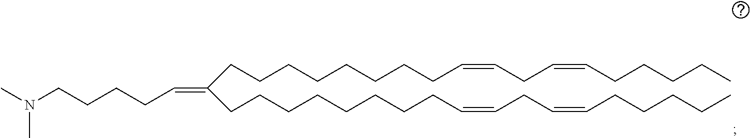 [SMILES: CCCCC/C=C\C/C=C\CCCCCCCCC(=CCCCCN(C)C)CCCCCCCC/C=C\C/C=C\CCCCC] |
| 7 | 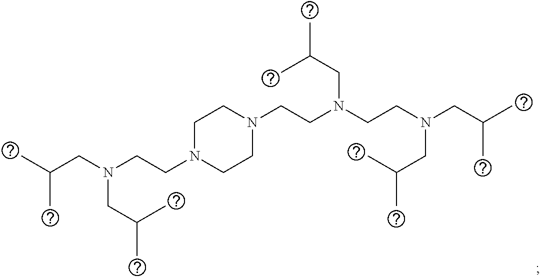 [SMILES: CC(C)CN(CCN1CCN(CCN(CC(C)C)CC(C)C)CC1)CCN(CC(C)C)CC(C)C] |
| 8 | 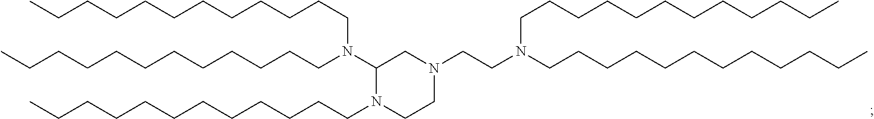 [SMILES: CCCCCCCCCCCCN(CCCCCCCCCCCC)CCN1CCN(CCCCCCCCCCCC)C(N(CCCCCCCCCCCC)CCCCCCCCCCCC)C1] |
| 9 | 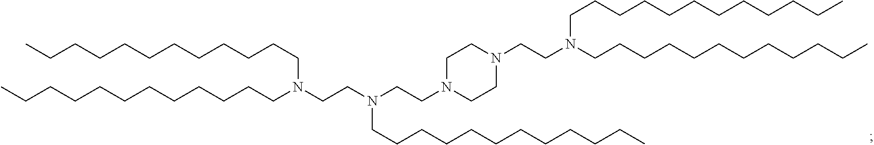 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1] |
| 10 | 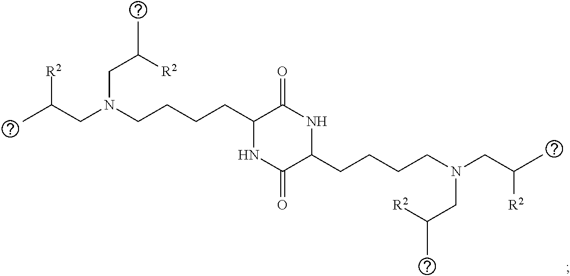 |
| 11 | 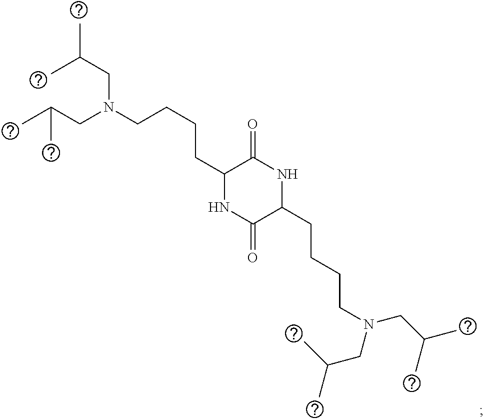 [SMILES: CC(C)CN(CCCCC1NC(=O)C(CCCCN(CC(C)C)CC(C)C)NC1=O)CC(C)C] |
| 12 | 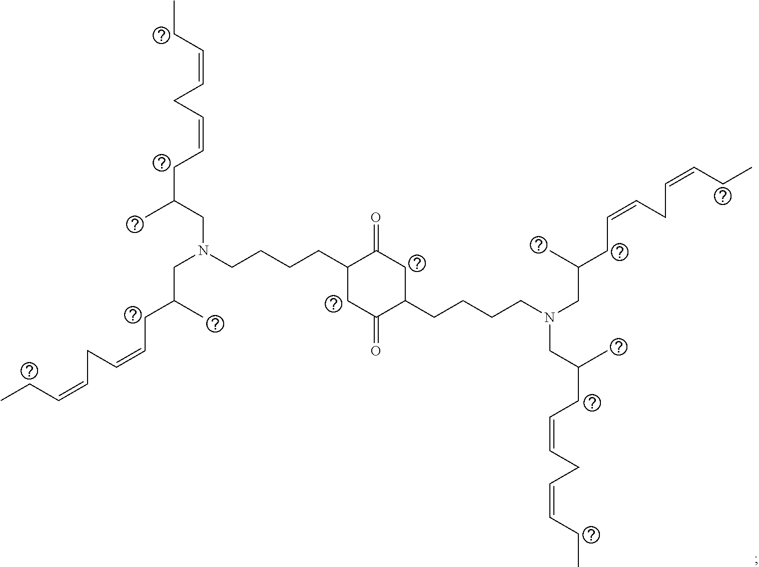 [SMILES: CC/C=C\C/C=C\CC(C)CN(CCCCC1CC(=O)C(CCCCN(CC(C)C/C=C\C/C=C\CC)CC(C)C/C=C\C/C=C\CC)CC1=O)CC(C)C/C=C\C/C=C\CC] |
| 13 | 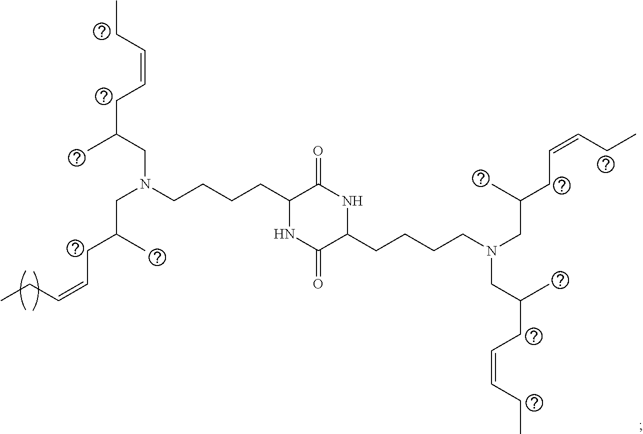 |
| 14 | 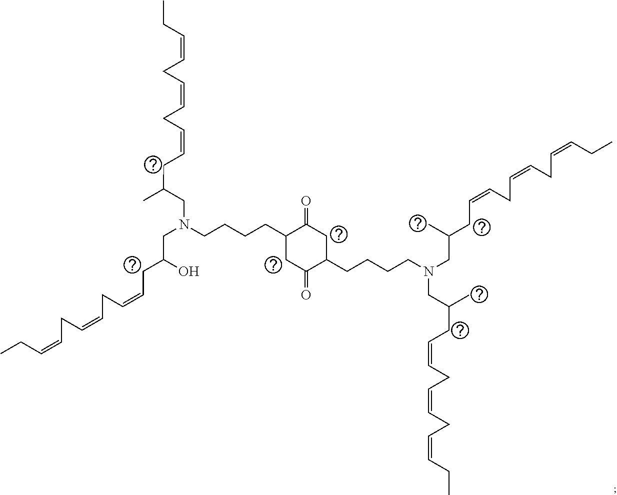 [SMILES: CC/C=C\C/C=C\C/C=C\CC(C)CN(CCCCC1CC(=O)C(CCCCN(CC(C)C/C=C\C/C=C\C/C=C\CC)CC(O)C/C=C\C/C=C\C/C=C\CC)CC1=O)CC(C)C/C=C\C/C=C\C/C=C\CC] |
| 15 | 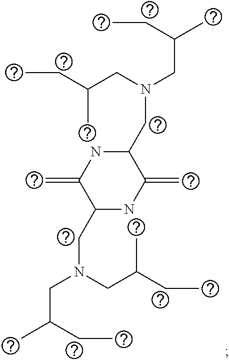 [SMILES: C=C1NC(CN(CC(C)CC)CC(C)CC)C(=C)NC1CN(CC(C)CC)CC(C)CC] |
| 16 | 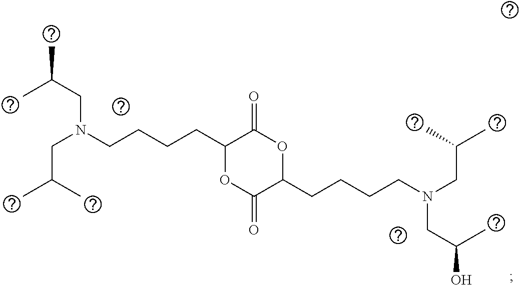 |
| 17 | 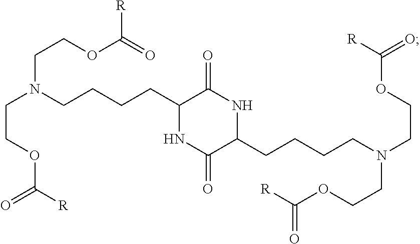 |
| 18 | 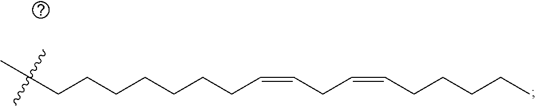 [SMILES: *CCCCCCC/C=C\C/C=C\CCCCC] |
| 19 | 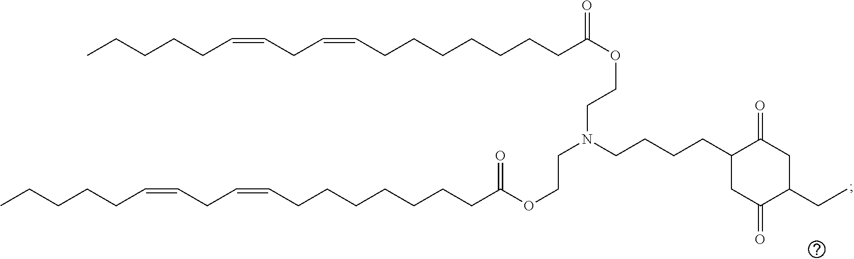 [SMILES: CCCCC/C=C\C/C=C\CCCCCCCC(=O)OCCN(CCCCC1CC(=O)C(CC)CC1=O)CCOC(=O)CCCCCCC/C=C\C/C=C\CCCCC] |
| 20 | 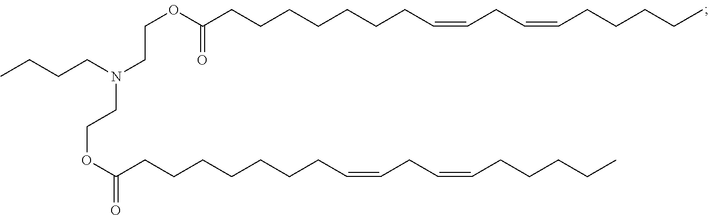 [SMILES: CCCCC/C=C\C/C=C\CCCCCCCC(=O)OCCN(CCCC)CCOC(=O)CCCCCCC/C=C\C/C=C\CCCCC] |
| 21 | 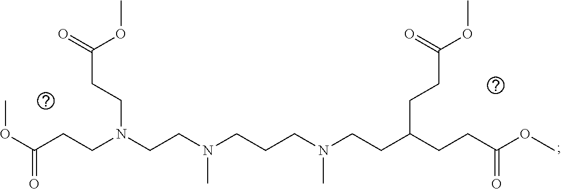 |
| 22 | 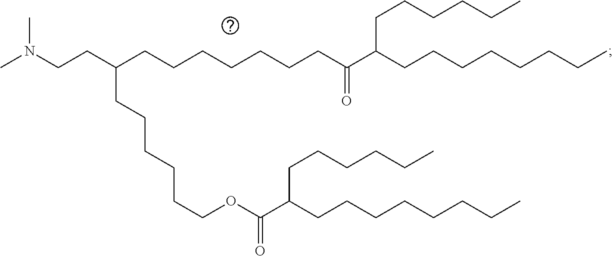 [SMILES: CCCCCCCCC(CCCCCC)C(=O)CCCCCCCC(CCCCCCOC(=O)C(CCCCCC)CCCCCCCC)CCN(C)C] |
| 23 | 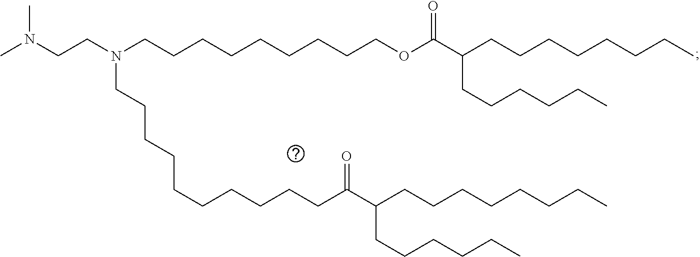 [SMILES: CCCCCCCCC(CCCCCC)C(=O)CCCCCCCCCCN(CCCCCCCCCOC(=O)C(CCCCCC)CCCCCCCC)CCN(C)C] |
| 24 | 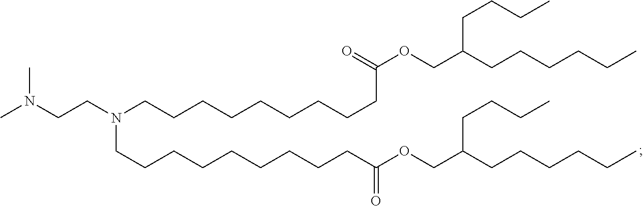 [SMILES: CCCCCCC(CCCC)COC(=O)CCCCCCCCCN(CCCCCCCCCC(=O)OCC(CCCC)CCCCCC)CCN(C)C] |
| 25 | 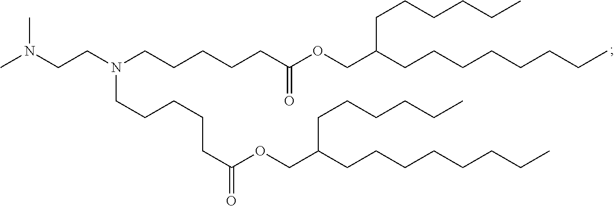 [SMILES: CCCCCCCCC(CCCCCC)COC(=O)CCCCCN(CCCCCC(=O)OCC(CCCCCC)CCCCCCCC)CCN(C)C] |
| 26 | 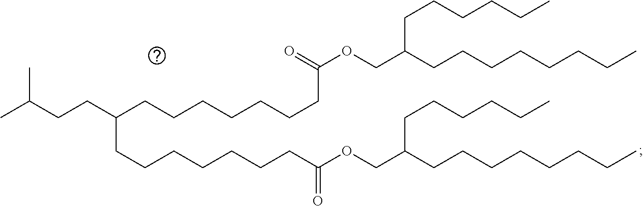 [SMILES: CCCCCCCCC(CCCCCC)COC(=O)CCCCCCCC(CCCCCCCC(=O)OCC(CCCCCC)CCCCCCCC)CCC(C)C] |
| 27 | 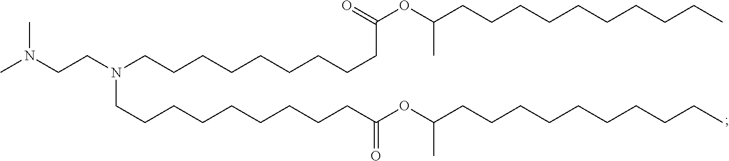 [SMILES: CCCCCCCCCCC(C)OC(=O)CCCCCCCCCN(CCCCCCCCCC(=O)OC(C)CCCCCCCCCC)CCN(C)C] |
| 28 | 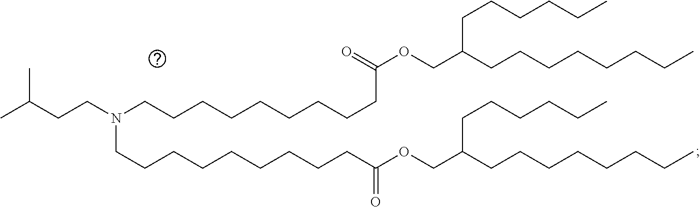 [SMILES: CCCCCCCCC(CCCCCC)COC(=O)CCCCCCCCCN(CCCCCCCCCC(=O)OCC(CCCCCC)CCCCCCCC)CCC(C)C] |
| 29 | 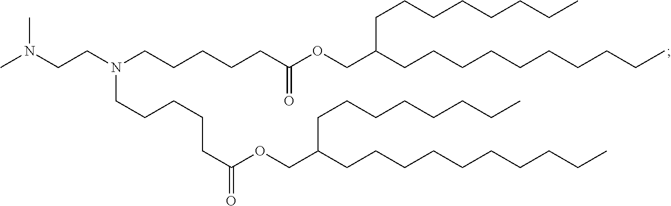 [SMILES: CCCCCCCCCCC(CCCCCCCC)COC(=O)CCCCCN(CCCCCC(=O)OCC(CCCCCCCC)CCCCCCCCCC)CCN(C)C] |
| 30 | 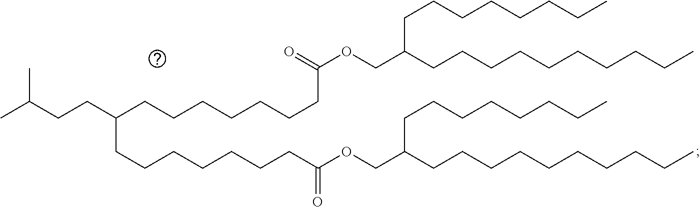 [SMILES: CCCCCCCCCCC(CCCCCCCC)COC(=O)CCCCCCCC(CCCCCCCC(=O)OCC(CCCCCCCC)CCCCCCCCCC)CCC(C)C] |
| 31 | 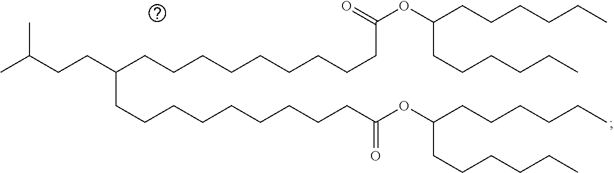 [SMILES: CCCCCCC(CCCCCC)OC(=O)CCCCCCCCCC(CCCCCCCCCC(=O)OC(CCCCCC)CCCCCC)CCC(C)C] |
| 32 | 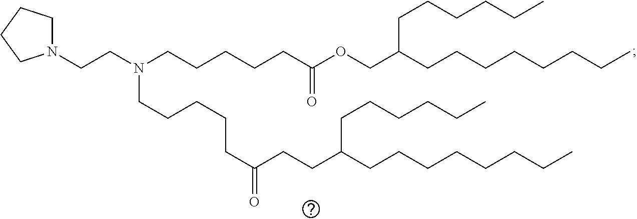 [SMILES: CCCCCCCCC(CCCCCC)CCC(=O)CCCCCN(CCCCCC(=O)OCC(CCCCCC)CCCCCCCC)CCN1CCCC1] |
| 33 | 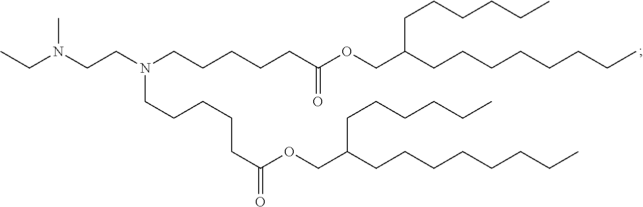 [SMILES: CCCCCCCCC(CCCCCC)COC(=O)CCCCCN(CCCCCC(=O)OCC(CCCCCC)CCCCCCCC)CCN(C)CC] |
| 34 | 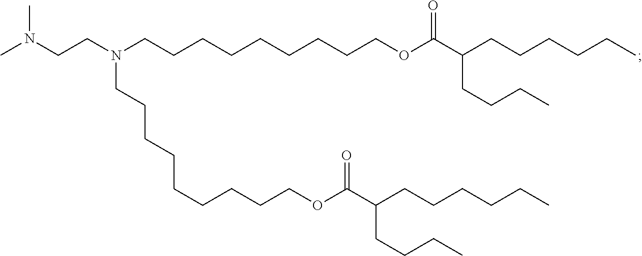 [SMILES: CCCCCCC(CCCC)C(=O)OCCCCCCCCCN(CCCCCCCCCOC(=O)C(CCCC)CCCCCC)CCN(C)C] |
| 35 | 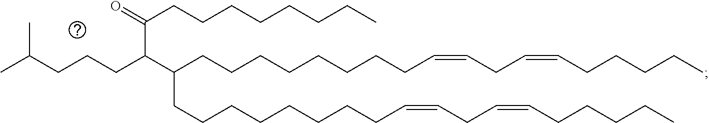 [SMILES: CCCCC/C=C\C/C=C\CCCCCCCCC(CCCCCCCC/C=C\C/C=C\CCCCC)C(CCCC(C)C)C(=O)CCCCCCCC] |
| 36 | 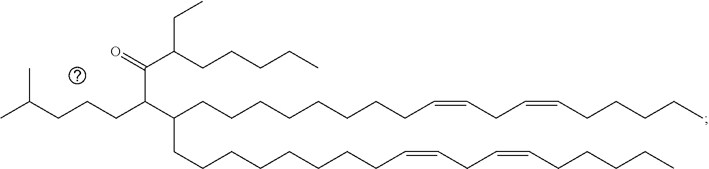 [SMILES: CCCCC/C=C\C/C=C\CCCCCCCCC(CCCCCCCC/C=C\C/C=C\CCCCC)C(CCCC(C)C)C(=O)C(CC)CCCCC] |
| 37 | 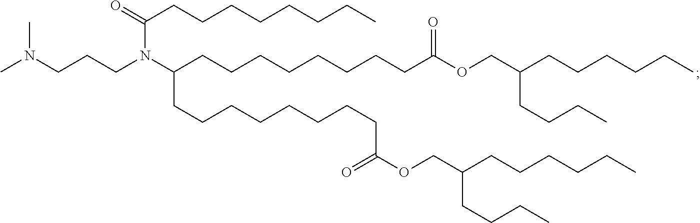 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC] |
| 38 | 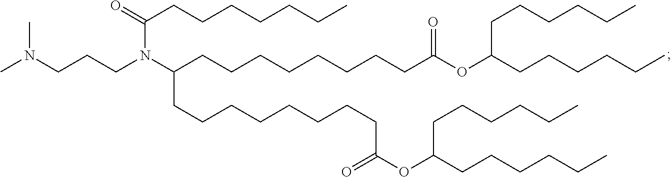 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |
| 39 | 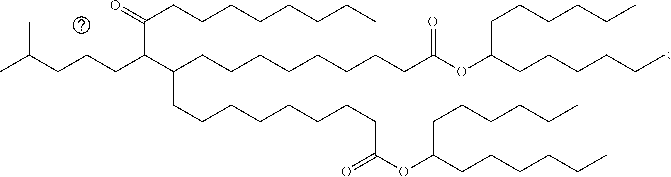 [SMILES: CCCCCCCCC(=O)C(CCCC(C)C)C(CCCCCCCCC(=O)OC(CCCCCC)CCCCCC)CCCCCCCCC(=O)OC(CCCCCC)CCCCCC] |
| 40 | 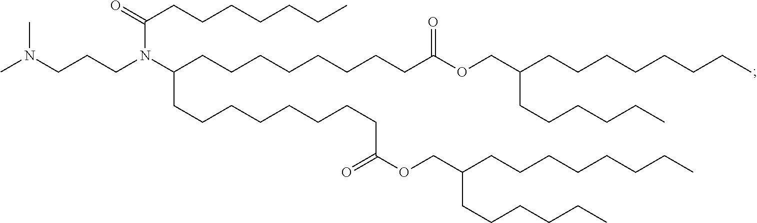 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC] |
| 41 | 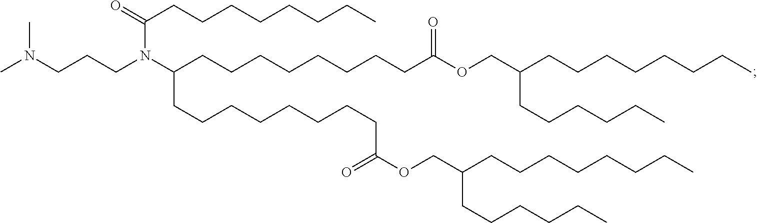 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC] |
| 42 | 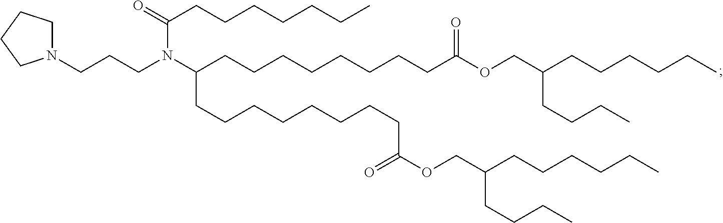 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC] |
| 43 | 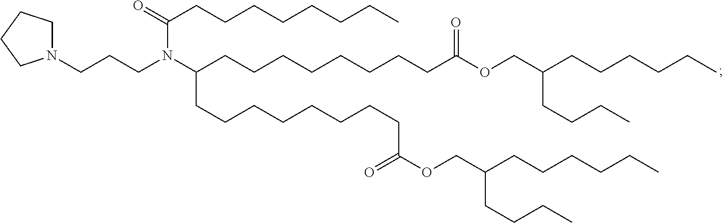 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC] |
| 44 | 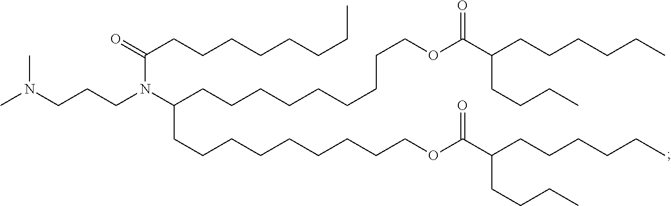 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC] |
| 45 | 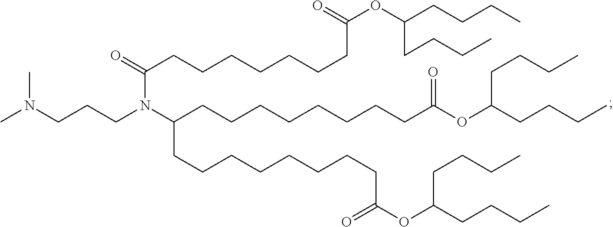 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC] |
| 46 | 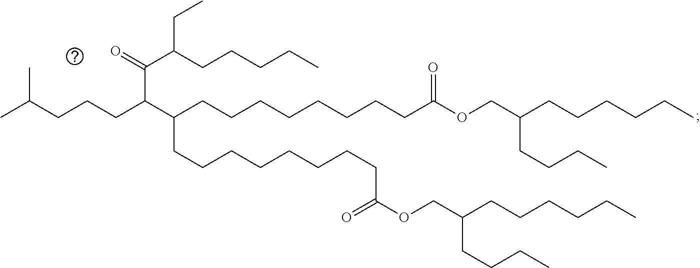 [SMILES: CCCCCCC(CCCC)COC(=O)CCCCCCCCC(CCCCCCCCC(=O)OCC(CCCC)CCCCCC)C(CCCC(C)C)C(=O)C(CC)CCCCC] |
| 47 | 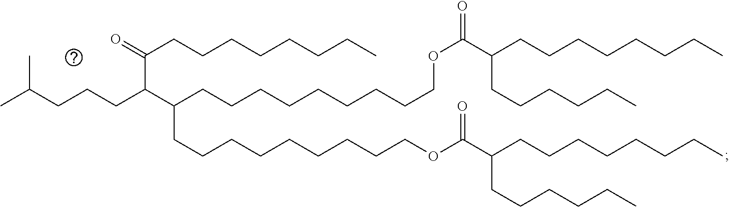 [SMILES: CCCCCCCCC(=O)C(CCCC(C)C)C(CCCCCCCCCOC(=O)C(CCCCCC)CCCCCCCC)CCCCCCCCCOC(=O)C(CCCCCC)CCCCCCCC] |
| 48 | 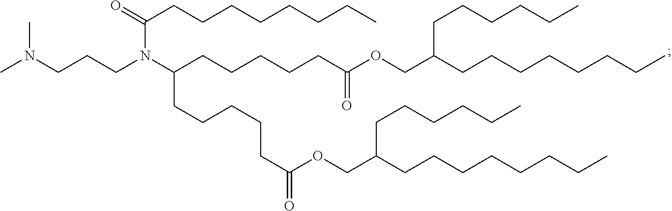 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC] |
| 49 | 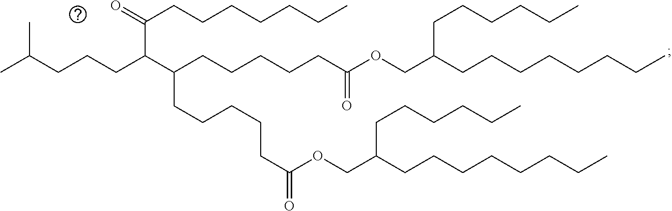 [SMILES: CCCCCCCCC(CCCCCC)COC(=O)CCCCCC(CCCCCC(=O)OCC(CCCCCC)CCCCCCCC)C(CCCC(C)C)C(=O)CCCCCCC] |
| 50 | 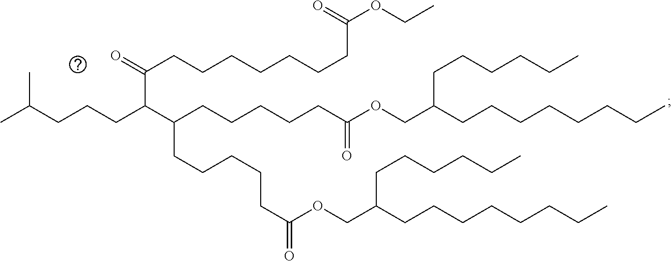 [SMILES: CCCCCCCCC(CCCCCC)COC(=O)CCCCCC(CCCCCC(=O)OCC(CCCCCC)CCCCCCCC)C(CCCC(C)C)C(=O)CCCCCCCC(=O)OCC] |
| 51 | 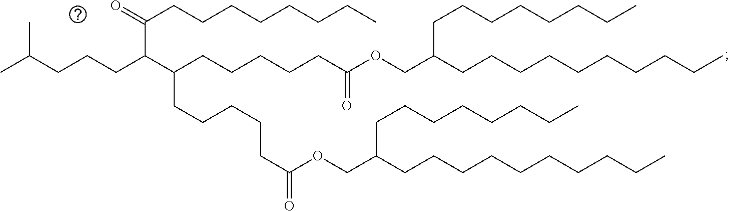 [SMILES: CCCCCCCCCCC(CCCCCCCC)COC(=O)CCCCCC(CCCCCC(=O)OCC(CCCCCCCC)CCCCCCCCCC)C(CCCC(C)C)C(=O)CCCCCCCC] |
| 52 | 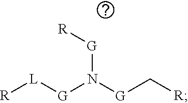 |
| 53 | 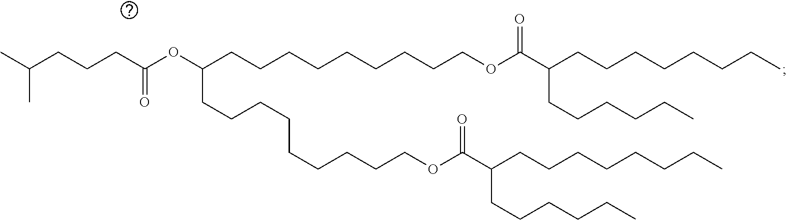 [SMILES: CCCCCCCCC(CCCCCC)C(=O)OCCCCCCCCCC(CCCCCCCCCOC(=O)C(CCCCCC)CCCCCCCC)OC(=O)CCCC(C)C] |
| 54 | 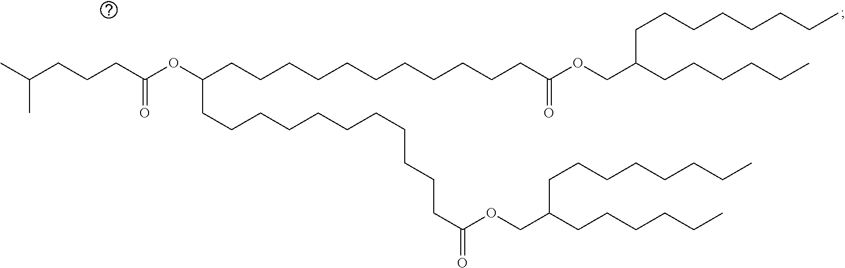 [SMILES: CCCCCCCCC(CCCCCC)COC(=O)CCCCCCCCCCCC(CCCCCCCCCCCC(=O)OCC(CCCCCC)CCCCCCCC)OC(=O)CCCC(C)C] |
| 55 | 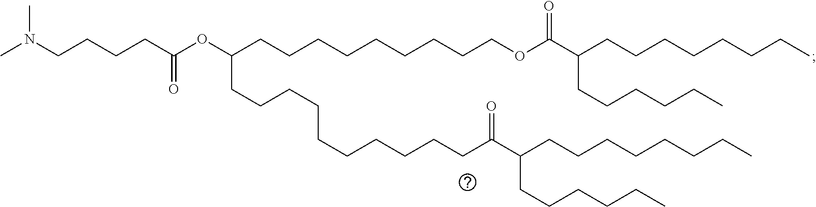 [SMILES: CCCCCCCCC(CCCCCC)C(=O)CCCCCCCCCCC(CCCCCCCCCOC(=O)C(CCCCCC)CCCCCCCC)OC(=O)CCCCN(C)C] |
| 56 | 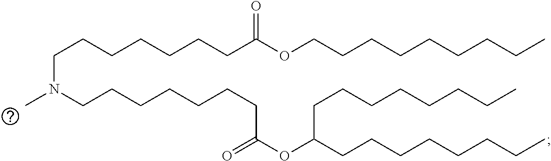 [SMILES: CCCCCCCCCOC(=O)CCCCCCCN(C)CCCCCCCC(=O)OC(CCCCCCCC)CCCCCCCC] |
| 57 | 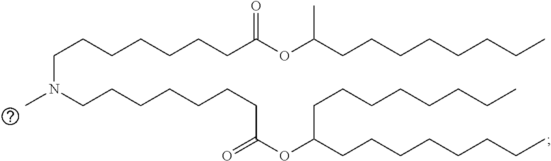 [SMILES: CCCCCCCCC(C)OC(=O)CCCCCCCN(C)CCCCCCCC(=O)OC(CCCCCCCC)CCCCCCCC] |
| 58 | 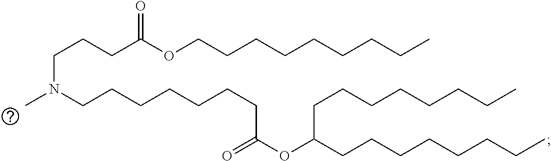 [SMILES: CCCCCCCCCOC(=O)CCCN(C)CCCCCCCC(=O)OC(CCCCCCCC)CCCCCCCC] |
| 59 | 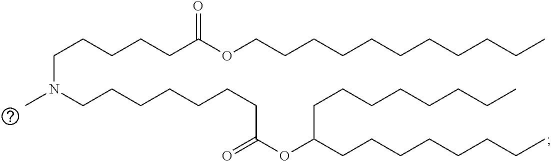 [SMILES: CCCCCCCCCCCOC(=O)CCCCCN(C)CCCCCCCC(=O)OC(CCCCCCCC)CCCCCCCC] |
| 60 | 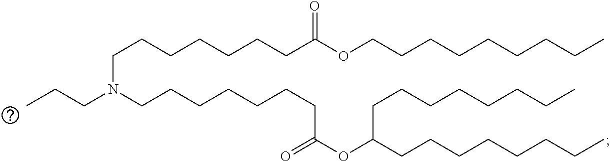 [SMILES: CCCCCCCCCOC(=O)CCCCCCCN(CCC)CCCCCCCC(=O)OC(CCCCCCCC)CCCCCCCC] |
| 61 | 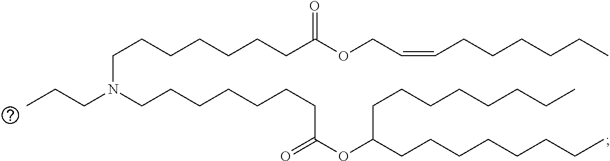 [SMILES: CCCCCC/C=C\COC(=O)CCCCCCCN(CCC)CCCCCCCC(=O)OC(CCCCCCCC)CCCCCCCC] |
| 62 | 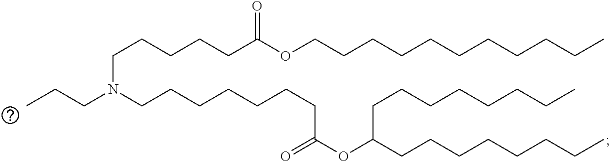 [SMILES: CCCCCCCCCCCOC(=O)CCCCCN(CCC)CCCCCCCC(=O)OC(CCCCCCCC)CCCCCCCC] |
| 63 | 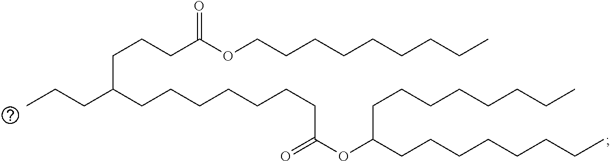 [SMILES: CCCCCCCCCOC(=O)CCCC(CCC)CCCCCCCC(=O)OC(CCCCCCCC)CCCCCCCC] |
| 64 | 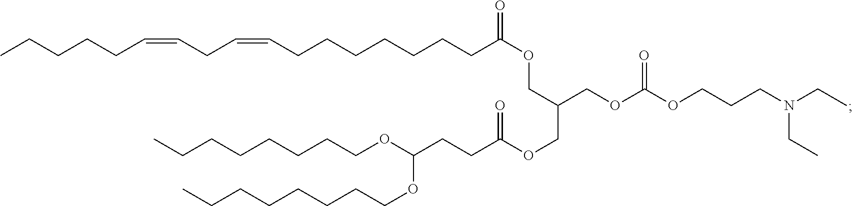 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| 65 | 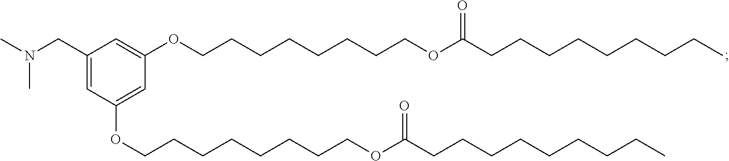 [SMILES: CCCCCCCCCC(=O)OCCCCCCCCOc1cc(CN(C)C)cc(OCCCCCCCCOC(=O)CCCCCCCCC)c1] |
| 66 | 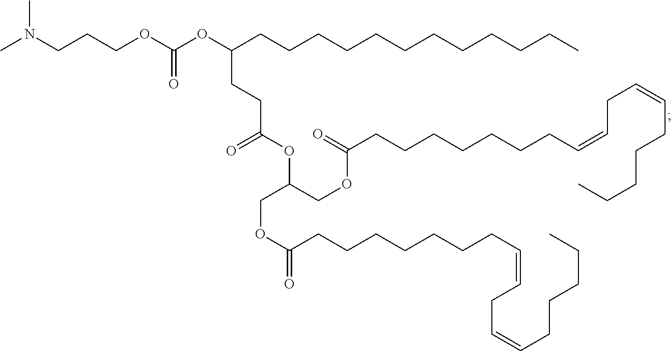 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C] |
| 67 | 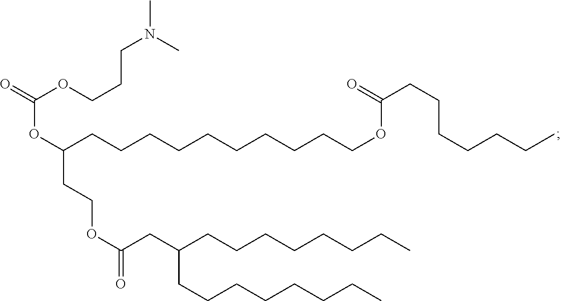 [SMILES: CCCCCCCCC(CCCCCCCC)CC(=O)OCCC(CCCCCCCCCCOC(=O)CCCCCCC)OC(=O)OCCCN(C)C] |
| 68 | 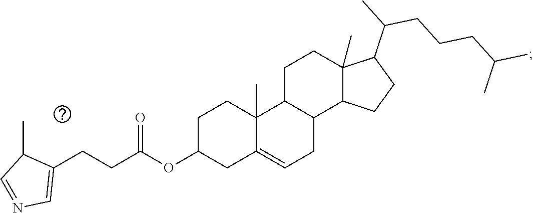 [SMILES: CC(C)CCCC(C)C1CCC2C3CC=C4CC(OC(=O)CCC5=CN=CC5C)CCC4(C)C3CCC12C] |
| 69 | 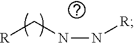 |
| 70 | 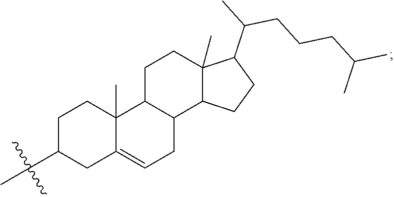 [SMILES: *C1CCC2(C)C(=CCC3C2CCC2(C)C(C(C)CCCC(C)C)CCC32)C1] |
| 71 | 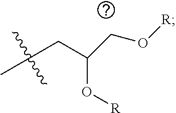 |
| 72 | 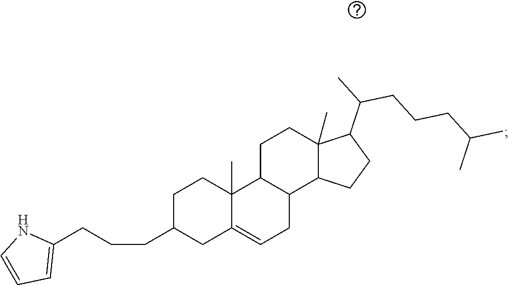 [SMILES: CC(C)CCCC(C)C1CCC2C3CC=C4CC(CCCc5ccc[nH]5)CCC4(C)C3CCC12C] |
| 73 | 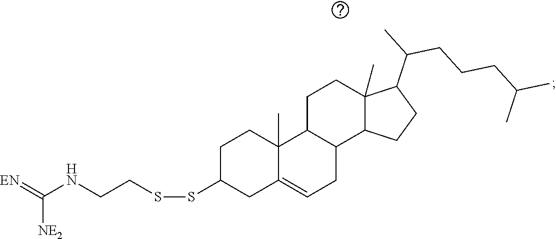 |
| 74 | 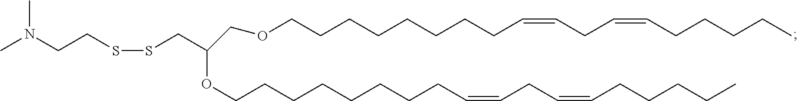 [SMILES: CCCCC/C=C\C/C=C\CCCCCCCCOCC(CSSCCN(C)C)OCCCCCCCC/C=C\C/C=C\CCCCC] |
| 75 | 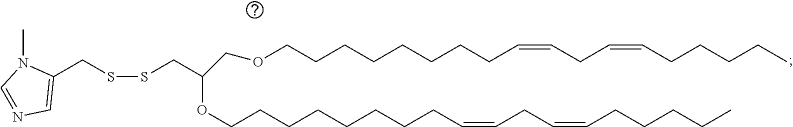 [SMILES: CCCCC/C=C\C/C=C\CCCCCCCCOCC(CSSCc1cncn1C)OCCCCCCCC/C=C\C/C=C\CCCCC] |
| 76 | 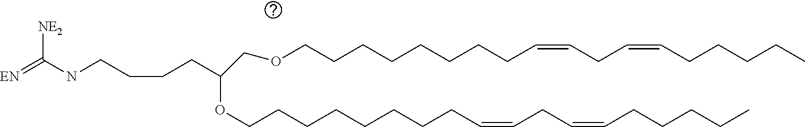 |

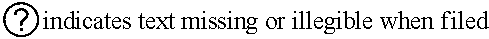

In some embodiments of the lipid composition of the present application, the ionizable cationic lipid is present in an amount from about from about 20 to about 23. In some embodiments, the molar percentage is from about 20, 20.5, 21, 21.5, 22, 22.5, to about 23 or any range derivable therein. In other embodiments, the molar percentage is from about 7.5 to about 20. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the ionizable cationic lipid at a molar percentage from about 5% to about 30%. In some embodiments of the lipid composition of the present application, the lipid composition comprises the ionizable cationic lipid at a molar percentage from about 10% to about 25%. In some embodiments of the lipid composition of the present application, the lipid composition comprises the ionizable cationic lipid at a molar percentage from about 15% to about 20%. In some embodiments of the lipid composition of the present application, the lipid composition comprises the ionizable cationic lipid at a molar percentage from about 10% to about 20%. In some embodiments of the lipid composition of the present application, the lipid composition comprises the ionizable cationic lipid at a molar percentage from about 20% to about 30%. In some embodiments of the lipid composition of the present application, the lipid composition comprises the ionizable cationic lipid at a molar percentage of at least (about) 5%, at least (about) 10%, at least (about) 15%, at least (about) 20%, at least (about) 25%, or at least (about) 30%. In some embodiments of the lipid composition of the present application, the lipid composition comprises the ionizable cationic lipid at a molar percentage of at most (about) 5%, at most (about) 10%, at most (about) 15%, at most (about) 20%, at most (about) 25%, or at most (about) 30%.

### Selective Organ Targeting (SORT) Lipids

In some embodiments of the lipid composition of the present application, the lipid (e.g., nanoparticle) composition is preferentially delivered to a target organ. In some embodiments, the target organ is a lung, a lung tissue or a lung cell. As used herein, the term “preferentially delivered” is used to refer to a composition, upon being delivered, which is delivered to the target organ (e.g., lung), tissue, or cell in at least 25% (e.g., at least 30%, 35%, 40%, 45%, 50%, 55%, 60%, 65%, 70%, or 75%) of the amount administered.

In some embodiments of the lipid composition, the lipid composition comprises one or more selective organ targeting (SORT) lipid which leads to the selective delivery of the composition to a particular organ. In some embodiments, the SORT lipid may have two or more alkyl or alkenyl chains of C₆-C₂₄.

In some embodiments of the lipid compositions, the SORT lipid comprises permanently positively charged moiety. The permanently positively charged moiety may be positively charged at a physiological pH such that the SORT lipid comprises a positive charge upon delivery of a polynucleotide to a cell. In some embodiments the positively charged moiety is quaternary amine or quaternary ammonium ion. In some embodiments, the SORT lipid comprises, or is otherwise complexed to or interacting with, a counterion.

In some embodiments of the lipid compositions, the SORT lipid is a permanently cationic lipid (i.e., comprising one or more hydrophobic components and a permanently cationic group). The permanently cationic lipid may contain a group which has a positive charge regardless of the pH. One permanently cationic group that may be used in the permanently cationic lipid is a quaternary ammonium group. The permanently cationic lipid may comprise a structural formula:

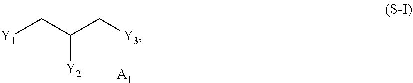

wherein:
Y₁, Y₂, or Y₃ are each independently X₁C(O)R₁ or X₂N⁺R₃R₄R₅;
provided at least one of Y₁, Y₂, and Y₃ is X₂N⁺R₃R₄R₅;
R₁ is C₁-C₂₄ alkyl, C₁-C₂₄ substituted alkyl, C₁-C₂₄ alkenyl, C₁-C₂₄ substituted alkenyl;
X₁ is O or NRₐ, wherein Rₐ is hydrogen, C₁-C₄ alkyl, or C₁-C₄ substituted alkyl;
X₂ is C₁-C₆ alkanediyl or C₁-C₆ substituted alkanediyl;
R₃, R₄, and R₅ are each independently C₁-C₂₄ alkyl, C₁-C₂₄ substituted alkyl, C₁-C₂₄ alkenyl, C₁-C₂₄ substituted alkenyl; and
A₁ is an anion with a charge equal to the number of X₂N⁺R₃R₄R₅ groups in the compound.

In some embodiments of the SORT lipids, the permanently cationic SORT lipid has a structural formula:

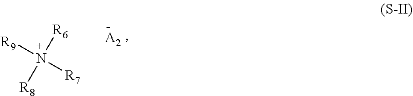

wherein:
R₆-R₉ are each independently C₁-C₂₄ alkyl, C₁-C₂₄ substituted alkyl, C₁-C₂₄ alkenyl, C₁-C₂₄ substituted alkenyl; provided at least one of R₆-R₉ is a group of C₈-C₂₄; and
A₂⁻ is a monovalent anion.

In some embodiments of the lipid compositions, the SORT lipid is ionizable cationic lipid (i.e., comprising one or more hydrophobic components and an ionizable cationic group). The ionizable positively charged moiety may be positively charged at a physiological pH. One ionizable cationic group that may be used in the ionizable cationic lipid is a tertiary ammine group. In some embodiments of the lipid compositions, the SORT lipid has a structural formula:

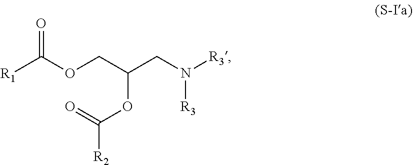

wherein:
R₁ and R₂ are each independently alkyl_{(C8-C24)}, alkenyl_{(C8-C24)}, or a substituted version of either group; and
R₃ and R₃′ are each independently alkyl_{(C≤6)} or substituted alkyl_{(C≤6)}.

In some embodiments of the lipid compositions, the SORT lipid comprises a head group of a particular structure. In some embodiments, the SORT lipid comprises a headgroup having a structural formula:

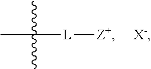

wherein L is a linker; Z⁺ is positively charged moiety and X⁻ is a counterion. In some embodiment, the linker is a biodegradable linker. The biodegradable linker may be degradable under physiological pH and temperature. The biodegradable linker may be degraded by proteins or enzymes from a subject. In some embodiments, the positively charged moiety is a quaternary ammonium ion or quaternary amine.

In some embodiments of the lipid compositions, the SORT lipid has a structural formula:

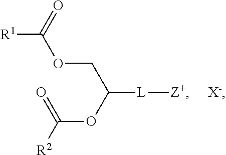

wherein R¹ and R² are each independently an optionally substituted C₆-C₂₄ alkyl, or an optionally substituted C₆-C₂₄ alkenyl.

In some embodiments of the lipid compositions, the SORT lipid has a structural formula:

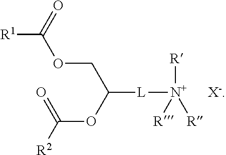

In some embodiments of the lipid compositions, the SORT lipid comprises a Linker (L). In some embodiments, L is

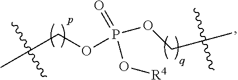

wherein:
p and q are each independently 1, 2, or 3; and
R⁴ is an optionally substituted C₁-C₆ alkyl

In some embodiments of the lipid compositions, the SORT lipid has a structural formula:

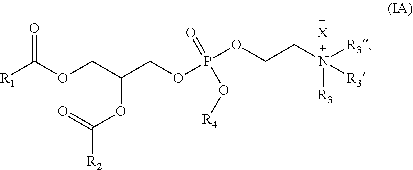

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
R₄ is alkyl_{(C≤6)} or substituted alkyl_{(C≤6)}; and
X⁻ is a monovalent anion.

In some embodiments of the lipid compositions, the SORT lipid is a phosphatidylcholine (e.g., 14:0 EPC). In some embodiments, the phosphatidylcholine compound is further defined as:

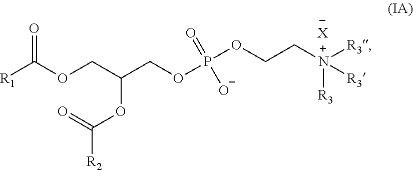

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In some embodiments of the lipid compositions, the SORT lipid is a phosphocholine lipid. In some embodiments, the SORT lipid is an ethylphosphocholine. The ethylphosphocholine may be, by way of example, without being limited to, 1,2-dimyristoleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1-palmitoyl-2-oleoyl-sn-glycero-3-ethylphosphocholine.

In some embodiments of the lipid compositions, the SORT lipid has a structural formula:

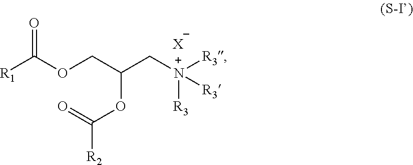

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)};
X⁻ is a monovalent anion.

By way of example, and without being limited thereto, a SORT lipid of the structural formula of the immediately preceding paragraph is 1,2-dioleoyl-3-trimethylammonium-propane (18:1 DOTAP) (e.g., chloride salt).

In some embodiments of the lipid compositions, the SORT lipid has a structural formula:

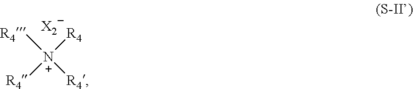

wherein:
R₄ and R₄′ are each independently alkyl_{(C6-C24)}, alkenyl_{(C6-C24)}, or a substituted version of either group;
R₄″ is alkyl_{(C≤24)}, alkenyl_{(C≤24)}, or a substituted version of either group;
R₄′″ is alkyl_{(C1-C8)}, alkenyl_{(C2-C8)}, or a substituted version of either group; and
X₂⁻ is a monovalent anion.

By way of example, and without being limited thereto, a SORT lipid of the structural formula of the immediately preceding paragraph is dimethyldioctadecylammonium (DDAB) (e.g., bromide salt).

In some embodiments of the lipid compositions, the SORT lipid has a structural formula:

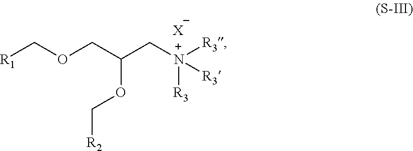

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

In some embodiments of the lipid compositions, the SORT lipid is an anionic lipid. In some embodiments of the lipid compositions, the SORT lipid has a structural formula:

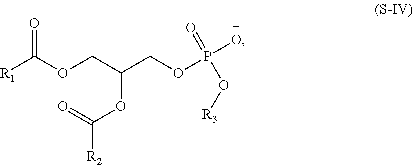

wherein:
R₁ and R₂ are each independently alkyl_{(C8-C24)}, alkenyl_{(C8-C24)}, or a substituted version of either group;
R₃ is hydrogen, alkyl_{(C≤6)}, or substituted alkyl_{(C≤6)}, or —Y₁—R₄, wherein:
   Y₁ is alkanediyl_{(C≤6)} or substituted alkanediyl_{(C≤6)}; and
R₄ is acyloxy_{(C≤8-24)} or substituted acyloxy_{(C≤8-24)}.

In some embodiments of the lipid compositions, the SORT lipid comprises one or more selected from the lipids set forth in Table 6.

TABLE 6
Example SORT lipids

| Lipid Name | Structure |
|---|---|
| 1,2-Dioleoyl-3- dimethylammonium-propane (18:1 DODAP) | 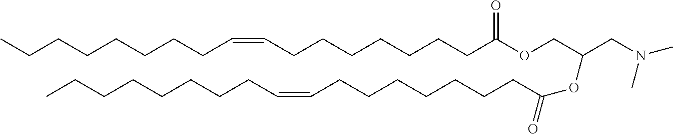 [SMILES: CCCCCCCC/C=C\CCCCCCCC(=O)OCC(CN(C)C)OC(=O)CCCCCCC/C=C\CCCCCCCC] |
| 1,2-dimyristoyl-3- trimethylammonium-propane (14:0 TAP) (e.g., chloride salt) | 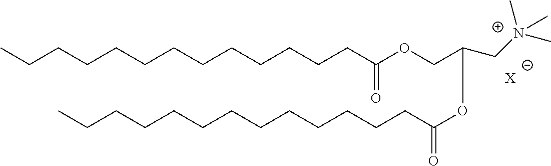 |
| 1,2-dipalmitoyl-3- trimethylammonium-propane (16:0 TAP) (e.g., chloride salt) | 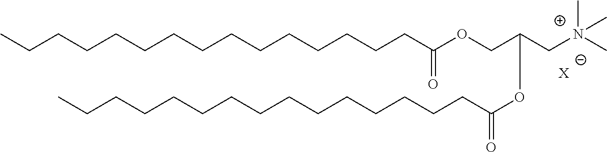 |
| 1,2-stearoyl-3- trimethylammonium-propane (18:0 TAP) (e.g., chloride salt) | 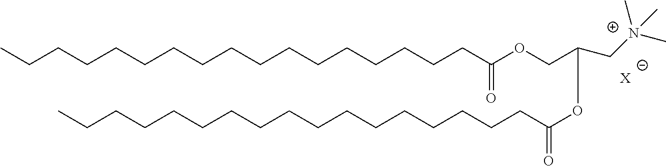 |
| 1,2-Dioleoyl-3- trimethylammonium-propane (18:1 DOTAP) (e.g., chloride salt) | 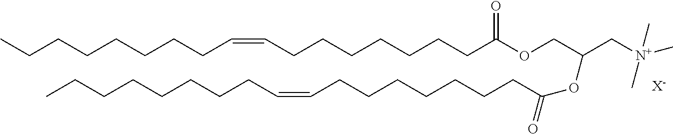 |
| 1,2-Di-O-octadeceny1-3- trimethylammonium propane (DOTMA) (e.g., chloride salt) | 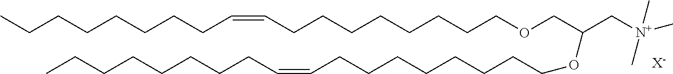 |
| Dimethyldioctadecylammonium (DDAB) (e.g., bromide salt) | 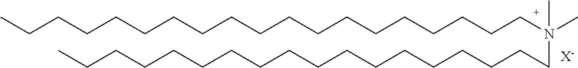 |
| 1,2-dilauroyl-sn-glycero-3- ethylphosphocholine (12:0 EPC) (e.g., chloride salt) | 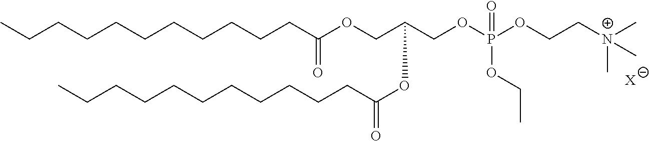 |
| 1,2-Dioleoyl-sn-glycero-3- ethylphosphocholine (14:0 EPC) (e.g., chloride salt) | 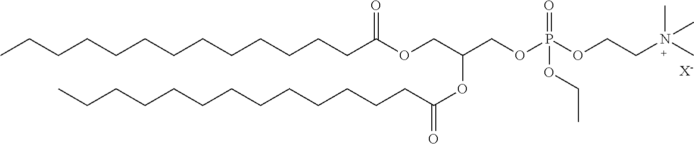 |
| 1,2-dimyristoleoyl-sn-glycero- 3-ethylphosphocholine (14:1 EPC) (e.g., triflate salt) | 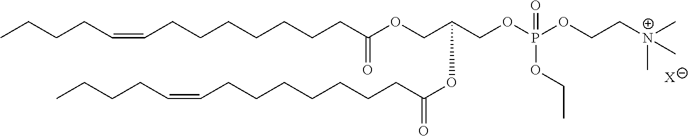 |
| 1,2-dipalmitoyl-sn-glycero-3- ethylphosphocholine (16:0 EPC) (e.g., chloride salt) | 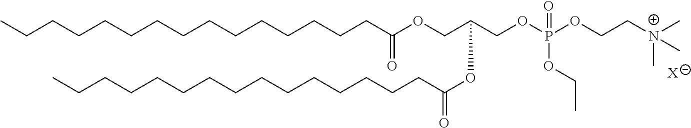 |
| 1,2-distearoyl-sn-glycero-3- ethylphosphocholine (18:0 EPC) (e.g., chloride salt) | 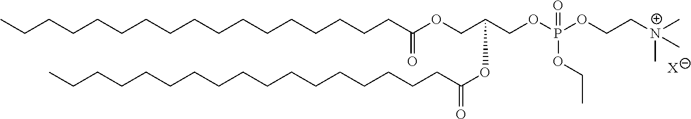 |
| 1,2-dioleoyl-sn-glycero-3- ethylphosphocholine (18:1 EPC) (e.g., chloride salt) | 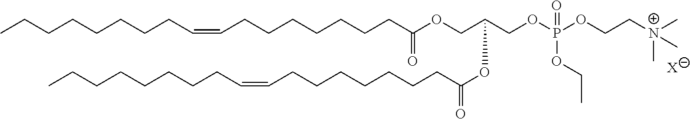 |
| 1-palmitoyl-2-oleoyl-sn- glycero-3-ethylphosphocholine (16:0-18:1 EPC) (e.g., chloride salt) | 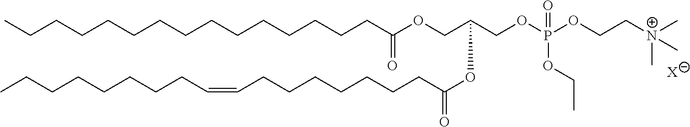 |
| 1,2-di-O-octadeceny1-3- trimethylammonium propane (18:1 DOTMA) (e.g., chloride salt) | 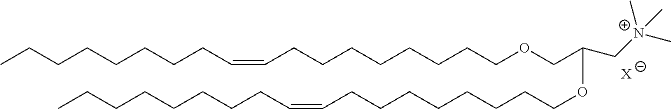 |
| 1,2-dioleoyl-sn-glycero-3- phosphate (18.1 PA) | 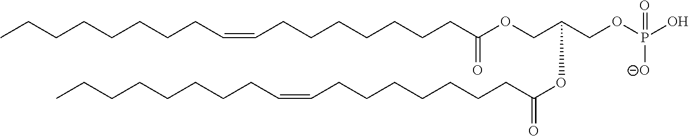 [SMILES: CCCCCCCC/C=C\CCCCCCCC(=O)OC[C@@H](COP(=O)([O-])O)OC(=O)CCCCCCC/C=C\CCCCCCCC] |

X⁻ is a counterion (e.g., Cl⁻, Br⁻, etc.)

In some embodiments of the lipid composition of the present application, the lipid composition comprises the SORT lipid at a molar percentage from about 20% to about 65%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage from about 30% to about 55%. In some embodiments of the lipid composition of the present application, the lipid composition comprises the SORT lipid at a molar percentage from about 20% to about 50%. In some embodiments of the lipid composition of the present application, the lipid composition comprises the SORT lipid at a molar percentage from about 30% to about 60%. In some embodiments of the lipid composition of the present application, the lipid composition comprises the SORT lipid at a molar percentage from about 25% to about 60%. In some embodiments of the lipid composition of the present application, the lipid composition comprises the SORT lipid at a molar percentage of at least (about) 25%, at least (about) 30%, at least (about) 35%, at least (about) 40%, at least (about) 45%, at least (about) 50%, at least (about) 55%, at least (about) 60%, or at least (about) 65%. In some embodiments of the lipid composition of the present application, the lipid composition comprises the SORT lipid at a molar percentage of at most (about) 25%, at most (about) 30%, at most (about) 35%, at most (about) 40%, at least (about) 45%, at most (about) 50%, at most (about) 55%, at most (about) 60%, or at most (about) 65%. In some embodiments of the lipid composition of the present application, the lipid composition comprises the SORT lipid at a molar percentage of about 20%, 25%, 30%, 35%, 40%, 45%, 50%, 55%, 60%, or 65%, or of a range between (inclusive) any two of the foregoing values.

### Additional Lipids

In some embodiments of the lipid composition of the present application, the lipid composition further comprises an additional lipid including but not limited to a steroid or a steroid derivative, a PEG lipid, and a phospholipid.

### Phospholipids or Other Zwitterionic Lipids

In some embodiments of the lipid composition of the present application, the lipid composition further comprises a phospholipid. In some embodiments, the phospholipid may contain one or two long chain (e.g., C₆-C₂₄) alkyl or alkenyl groups, a glycerol or a sphingosine, one or two phosphate groups, and, optionally, a small organic molecule. The small organic molecule may be an amino acid, a sugar, or an amino substituted alkoxy group, such as choline or ethanolamine. In some embodiments, the phospholipid is a phosphatidylcholine. In some embodiments, the phospholipid is distearoylphosphatidylcholine or dioleoylphosphatidylethanolamine. In some embodiments, other zwitterionic lipids are used, where zwitterionic lipid defines lipid and lipid-like molecules with both a positive charge and a negative charge.

In some embodiments of the lipid compositions, the phospholipid is not an ethylphosphocholine.

In some embodiments of the lipid composition of the present application, the compositions may further comprise a molar percentage of the phospholipid to the total lipid composition from about 20 to about 23. In some embodiments, the molar percentage is from about 20, 20.5, 21, 21.5, 22, 22.5, to about 23 or any range derivable therein. In other embodiments, the molar percentage is from about 7.5 to about 60. In some embodiments, the molar percentage is from about 7.5, 8, 9, 10, 11, 12, 13, 14, 15, 16, 17, 18, 19, to about 20 or any range derivable therein.

In some embodiments of the lipid composition of the present application, the lipid composition comprises the phospholipid at a molar percentage from about 8% to about 23%. In some embodiments of the lipid composition of the present application, the lipid composition comprises the phospholipid at a molar percentage from about 10% to about 20%. In some embodiments of the lipid composition of the present application, the lipid composition comprises the phospholipid at a molar percentage from about 15% to about 20%. In some embodiments of the lipid composition of the present application, the lipid composition comprises the phospholipid at a molar percentage from about 8% to about 15%. In some embodiments of the lipid composition of the present application, the lipid composition comprises the phospholipid at a molar percentage from about 10% to about 15%. In some embodiments of the lipid composition of the present application, the lipid composition comprises the phospholipid at a molar percentage from about 12% to about 18%. In some embodiments of the lipid composition of the present application, the lipid composition comprises the phospholipid at a molar percentage of at least (about) 8%, at least (about) 10%, at least (about) 12%, at least (about) 15%, at least (about) 18%, at least (about) 20%, or at least (about) 23%. In some embodiments of the lipid composition of the present application, the lipid composition comprises the phospholipid at a molar percentage of at most (about) 8%, at most (about) 10%, at most (about) 12%, at most (about) 15%, at most (about) 18%, at most (about) 20%, or at most (about) 23%.

### Steroids or Steroid Derivatives

In some embodiments of the lipid composition of the present application, the lipid composition further comprises a steroid or steroid derivative. In some embodiments, the steroid or steroid derivative comprises any steroid or steroid derivative. As used herein, in some embodiments, the term “steroid” is a class of compounds with a four ring 17 carbon cyclic structure which can further comprises one or more substitutions including alkyl groups, alkoxy groups, hydroxy groups, oxo groups, acyl groups, or a double bond between two or more carbon atoms. In one aspect, the ring structure of a steroid comprises three fused cyclohexyl rings and a fused cyclopentyl ring as shown in the formula:

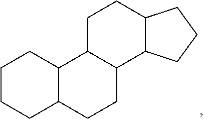 [SMILES: C1CCC2C(C1)CCC1C3CCCC3CCC21]

In some embodiments, a steroid derivative comprises the ring structure above with one or more non-alkyl substitutions. In some embodiments, the steroid or steroid derivative is a sterol wherein the formula is further defined as:

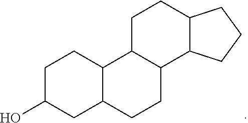 [SMILES: OC1CCC2C(CCC3C4CCCC4CCC23)C1]

In some embodiments of the present application, the steroid or steroid derivative is a cholestane or cholestane derivative. In a cholestane, the ring structure is further defined by the formula:

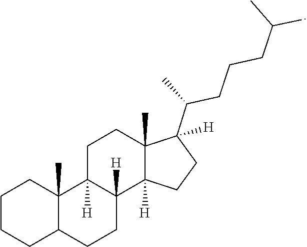

As described above, a cholestane derivative includes one or more non-alkyl substitution of the above ring system. In some embodiments, the cholestane or cholestane derivative is a cholestene or cholestene derivative or a sterol or a sterol derivative. In other embodiments, the cholestane or cholestane derivative is both a cholestere and a sterol or a derivative thereof.

In some embodiments of the lipid composition, the compositions may further comprise a molar percentage of the steroid to the total lipid composition from about 40 to about 46. In some embodiments, the molar percentage is from about 40, 41, 42, 43, 44, 45, to about 46 or any range derivable therein. In other embodiments, the molar percentage of the steroid relative to the total lipid composition is from about 15 to about 40. In some embodiments, the molar percentage is 15, 16, 18, 20, 22, 24, 26, 28, 30, 32, 34, 36, 38, or 40, or any range derivable therein.

In some embodiments of the lipid composition of the present application, the lipid composition comprises the steroid or steroid derivative at a molar percentage from about 15% to about 46%. In some embodiments of the lipid composition of the present application, the lipid composition comprises the steroid or steroid derivative at a molar percentage from about 20% to about 40%. In some embodiments of the lipid composition of the present application, the lipid composition comprises the steroid or steroid derivative at a molar percentage from about 25% to about 35%. In some embodiments of the lipid composition of the present application, the lipid composition comprises the steroid or steroid derivative at a molar percentage from about 30% to about 40%. In some embodiments of the lipid composition of the present application, the lipid composition comprises the steroid or steroid derivative at a molar percentage from about 20% to about 30%. In some embodiments of the lipid composition of the present application, the lipid composition comprises the steroid or steroid derivative at a molar percentage of at least (about) 15%, of at least (about) 20%, of at least (about) 25%, of at least (about) 30%, of at least (about) 35%, of at least (about) 40%, of at least (about) 45%, or of at least (about) 46%. In some embodiments of the lipid composition of the present application, the lipid composition comprises the steroid or steroid derivative at a molar percentage of at most (about) 15%, of at most (about) 20%, of at most (about) 25%, of at most (about) 30%, of at most (about) 35%, of at most (about) 40%, of at most (about) 45%, or of at most (about) 46%.

### Polymer-Conjugated Lipids

In some embodiments of the lipid composition of the present application, the lipid composition further comprises a polymer conjugated lipid. In some embodiments, the polymer conjugated lipid is a PEG lipid. In some embodiments, the PEG lipid is a diglyceride which also comprises a PEG chain attached to the glycerol group. In other embodiments, the PEG lipid is a compound which contains one or more C₆-C₂₄ long chain alkyl or alkenyl group or a C₆-C₂₄ fatty acid group attached to a linker group with a PEG chain. Some non-limiting examples of a PEG lipid includes a PEG modified phosphatidylethanolamine and phosphatidic acid, a PEG ceramide conjugated, PEG modified dialkylamines and PEG modified 1,2-diacyloxypropan-3-amines, PEG modified diacylglycerols and dialkylglycerols. In some embodiments, PEG modified diastearoylphosphatidylethanolamine or PEG modified dimyristoyl-sn-glycerol. In some embodiments, the PEG modification is measured by the molecular weight of PEG component of the lipid. In some embodiments, the PEG modification has a molecular weight from about 100 to about 15,000. In some embodiments, the molecular weight is from about 200 to about 500, from about 400 to about 5,000, from about 500 to about 3,000, or from about 1,200 to about 3,000. The molecular weight of the PEG modification is from about 100, 200, 400, 500, 600, 800, 1,000, 1,250, 1,500, 1,750, 2,000, 2,250, 2,500, 2,750, 3,000, 3,500, 4,000, 4,500, 5,000, 6,000, 7,000, 8,000, 9,000, 10,000, 12,500, to about 15,000. Some non-limiting examples of lipids that may be used in the present application are taught by U.S. Pat. No. 5,820,873, WO 2010/141069, or U.S. Pat. No. 8,450,298, which is incorporated herein by reference.

In some embodiments of the lipid composition of the present application, the PEG lipid has a structural formula:

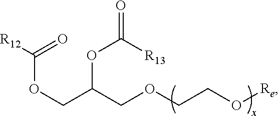

wherein: R₁₂ and R₁₃ are each independently alkyl_{(C≤24)}, alkenyl_{(C≤24)}, or a substituted version of either of these groups; Rₑ is hydrogen, alkyl_{(C≤8)}, or substituted alkyl_{(C≤8)}; and x is 1-250. In some embodiments, Rₑ is alkyl_{(C≤18)} such as methyl. R₁₂ and R₁₃ are each independently alkyl_{(C≤4-20)}. In some embodiments, x is 5-250. In one embodiment, x is 5-125 or x is 100-250. In some embodiments, the PEG lipid is 1,2-dimyristoyl-sn-glycerol, methoxypolyethylene glycol.

In some embodiments of the lipid composition of the present application, the PEG lipid has a structural formula:

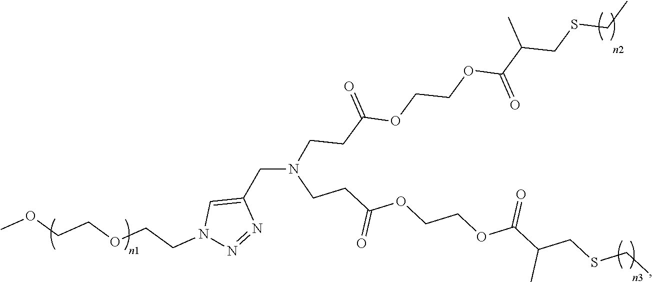

wherein: n₁ is an integer between 1 and 100 and n₂ and n₃ are each independently selected from an integer between 1 and 29. In some embodiments, n₁ is 5, 10, 15, 20, 25, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100, or any range derivable therein. In some embodiments, n₁ is from about 30 to about 50. In some embodiments, n₂ is from 5 to 23. In some embodiments, n₂ is 11 to about 17. In some embodiments, n₃ is from 5 to 23. In some embodiments, n₃ is 11 to about 17.

In some embodiments of the lipid composition of the present application, the compositions may further comprise a molar percentage of the PEG lipid to the total lipid composition from about 4.0 to about 4.6. In some embodiments, the molar percentage is from about 4.0, 4.1, 4.2, 4.3, 4.4, 4.5, to about 4.6 or any range derivable therein. In other embodiments, the molar percentage is from about 1.5 to about 4.0. In some embodiments, the molar percentage is from about 1.5, 1.75, 2, 2.25, 2.5, 2.75, 3, 3.25, 3.5, 3.75, to about 4.0 or any range derivable therein.

In some embodiments of the lipid composition of the present application, the lipid composition comprises the polymer-conjugated lipid at a molar percentage from about 0.5% to about 10%. In some embodiments of the lipid composition of the present application, the lipid composition comprises the polymer-conjugated lipid at a molar percentage from about 1% to about 8%. In some embodiments of the lipid composition of the present application, the lipid composition comprises the polymer-conjugated lipid at a molar percentage from about 2% to about 7%. In some embodiments of the lipid composition of the present application, the lipid composition comprises the polymer-conjugated lipid at a molar percentage from about 3% to about 5%. In some embodiments of the lipid composition of the present application, the lipid composition comprises the polymer-conjugated lipid at a molar percentage from about 5% to about 10%. In some embodiments of the lipid composition of the present application, the lipid composition comprises the polymer-conjugated lipid at a molar percentage of at least (about) 0.5%, at least (about) 1%, at least (about) 1.5%, at least (about) 2%, at least (about) 2.5%, at least (about) 3%, at least (about) 3.5%, at least (about) 4%, at least (about) 4.5%, at least (about) 5%, at least (about) 5.5%, at least (about) 6%, at least (about) 6.5%, at least (about) 7%, at least (about) 7.5%, at least (about) 8%, at least (about) 8.5%, at least (about) 9%, at least (about) 9.5%, or at least (about) 10%. In some embodiments of the lipid composition of the present application, the lipid composition comprises the polymer-conjugated lipid at a molar percentage of at most (about) 0.5%, at most (about) 1%, at most (about) 1.5%, at most (about) 2%, at most (about) 2.5%, at most (about) 3%, at most (about) 3.5%, at most (about) 4%, at most (about) 4.5%, at most (about) 5%, at most (about) 5.5%, at most (about) 6%, at most (about) 6.5%, at most (about) 7%, at most (about) 7.5%, at most (about) 8%, at most (about) 8.5%, at most (about) 9%, at most (about) 9.5%, or at most (about) 10%.

### Pharmaceutical Compositions

### Therapeutic or Prophylactic Agents

In another aspect, provided herein is a pharmaceutical composition comprising a therapeutic agent (or prophylactic agent) assembled with a lipid composition as described herein.

In some embodiments of the pharmaceutical composition, the therapeutic agent (or prophylactic agent) comprises a compound, a polynucleotide, a polypeptide, or a combination thereof. In some embodiments, the compound, the polynucleotide, the polypeptide, or a combination thereof is exogenous or heterologous to the cell or the subject being treated by the pharmaceutical compositions described herein. In some embodiments, the therapeutic agent (or prophylactic agent) comprises a compound described herein. In some embodiments, the therapeutic agent (or prophylactic agent) comprises a polynucleotide described herein. In some embodiments, the therapeutic agent (or prophylactic agent) comprises a polypeptide described herein. In some embodiments, the therapeutic agent (or prophylactic agent) comprises a compound, a polynucleotide, a polypeptide, or a combination thereof.

In some embodiments, the pharmaceutical composition comprises a therapeutic agent (or prophylactic agent) for treating a lung disease such as asthma, COPD, or lung cancer. In some embodiments, the therapeutic agent (or prophylactic agent) comprises a steroid such as prednisone, hydrocortisone, prednisolone, methylprednisolone, or dexamethasone. In some embodiments, the therapeutic agent (or prophylactic agent) comprises Abraxane, Afatinib Dimaleate, Afinitor, Afinitor Disperz, Alecensa, Alectinib, Alimta, Alunbrig, Atezolizumab, Avastin, Bevacizumab, Brigatinib, Capmatinib Hydrochloride, Carboplatin, Ceritinib, Crizotinib, Cyramza, Dabrafenib Mesylate, Dacomitinib, Docetaxel, Doxorubicin Hydrochloride, Durvalumab, Entrectinib, Erlotinib Hydrochloride, Everolimus, Gavreto, Gefitinib, Gilotrif, Gemcitabine, Ipilimumab, Iressa, Keytruda, Lorbrena, Mekinist, Methotrexate Sodium, Necitumumab, Nivolumab, Osimertinib Mesylate, Paclitaxel, Pembrolizumab, Pemetrexed Disodium, Pralsetinib, Ramucirumab, Retevmo, Selpercatinib, Tabrecta, Tafinlar, Tagrisso, Trametinib Dimethyl Sulfoxide, Vizimpro, Vinorelbine Tartrate, Xalkori, Yervoy, Zirabev, Zykadia, Carboplatin, Gemcitabine-cisplatin, Afinitor, Atezolizumab, Durvalumab, Etopophos, Etoposide, Hycamtin, Imfinzi, Keytruda, Lurbinectedin, Methotrexate Sodium, Nivolumab, Opdivo, Pembrolizumab, Tecentriq, Topotecan Hydrochloride, Trexall, or Zepzelca. Other non-limiting examples of the therapeutic agents (or prophylactic agents) comprising compounds include small molecule selected from 7-Methoxypteridine, 7 Methylpteridine, abacavir, abafungin, abarelix, acebutolol, acenaphthene, acetaminophen, acetanilide, acetazolamide, acetohexamide, acetretin, acrivastine, adenine, adenosine, alatrofloxacin, albendazole, albuterol, alclofenac, aldesleukin, alemtuzumab, alfuzosin, alitretinoin, allobarbital, allopurinol, all-transretinoic acid (ATRA), aloxiprin, alprazolam, alprenolol, altretamine, amifostine, amiloride, aminoglutethimide, aminopyrine, amiodarone HCl, amitriptyline, amlodipine, amobarbital, amodiaquine, amoxapine, amphetamine, amphotericin, amphotericin B, ampicillin, amprenavir, amsacrine, amylnitrate, amylobarbitone, anastrozole, anrinone, anthracene, anthracyclines, aprobarbital, arsenic trioxide, asparaginase, aspirin, astemizole, atenolol, atorvastatin, atovaquone, atrazine, atropine, atropine azathioprine, auranofin, azacitidine, azapropazone, azathioprine, azintamide, azithromycin, aztreonum, baclofen, barbitone, BCG live, beclamide, beclomethasone, bendroflumethiazide, benezepril, benidipine, benorylate, benperidol, bentazepam, benzamide, benzanthracene, benzathine penicillin, benzhexol HCl, benznidazole, benzodiazepines, benzoic acid, bephenium hydroxynaphthoate, betamethasone, bevacizumab (avastin), bexarotene, bezafibrate, bicalutamide, bifonazole, biperiden, bisacodyl, bisantrene, bleomycin, bleomycin, bortezomib, brinzolamide, bromazepam, bromocriptine mesylate, bromperidol, brotizolam, budesonide, bumetanide, bupropion, busulfan, butalbital, butamben, butenafine HCl, butobarbitone, butobarbitone (butethal), butoconazole, butoconazole nitrate, butylparaben, caffeine, calcifediol, calciprotriene, calcitriol, calusterone, cambendazole, camphor, camptothecin, camptothecin analogs, candesartan, capecitabine, capsaicin, captopril, carbamazepine, carbimazole, carbofuran, carboplatin, carbromal, carimazole, carmustine, cefamandole, cefazolin, cefixime, ceftazidime, cefuroxime axetil, celecoxib, cephradine, cerivastatin, cetrizine, cetuximab, chlorambucil, chloramphenicol, chlordiazepoxide, chlormethiazole, chloroquine, chlorothiazide, chlorpheniramine, chlorproguanil HCl, chlorpromazine, chlorpropamide, chlorprothixene, chlorpyrifos, chlortetracycline, chlorthalidone, chlorzoxazone, cholecalciferol, chrysene, cilostazol, cimetidine, cinnarizine, cinoxacin, ciprofibrate, ciprofloxacin HCl, cisapride, cisplatin, citalopram, cladribine, clarithromycin, clemastine fumarate, clioquinol, clobazam, clofarabine, clofazimine, clofibrate, clomiphene citrate, clomipramine, clonazepam, clopidogrel, clotiazepam, clotrimazole, clotrimazole, cloxacillin, clozapine, cocaine, codeine, colchicine, colistin, conjugated estrogens, corticosterone, cortisone, cortisone acetate, cyclizine, cyclobarbital, cyclobenzaprine, cyclobutane-spirobarbiturate, cycloethane-spirobarbiturate, cycloheptane-spirobarbiturate, cyclohexane-spirobarbiturate, cyclopentane-spirobarbiturate, cyclophosphamide, cyclopropane-spirobarbiturate, cycloserine, cyclosporin, cyproheptadine, cytarabine, cytosine, dacarbazine, dactinomycin, danazol, danthron, dantrolene sodium, dapsone, darbepoetin alfa, darodipine, daunorubicin, decoquinate, dehydroepiandrosterone, delavirdine, demeclocycline, denileukin, deoxycorticosterone, desoxymethasone, dexamethasone, dexamphetamine, dexchlorpheniramine, dexfenfluramine, dexrazoxane, dextropropoxyphene, diamorphine, diatrizoicacid, diazepam, diazoxide, dichlorophen, dichlorprop, diclofenac, dicumarol, didanosine, diflunisal, digitoxin, digoxin, dihydrocodeine, dihydroequilin, dihydroergotamine mesylate, diiodohydroxyquinoline, diltiazem HCl, diloxamide furoate, dimenhydrinate, dimorpholamine, dinitolmide, diosgenin, diphenoxylate HCl, diphenyl, dipyridamole, dirithromycin, disopyramide, disulfiram, diuron, docetaxel, domperidone, donepezil, doxazosin, doxazosin HCl, doxorubicin, doxycycline, dromostanolone propionate, droperidol, dyphylline, echinocandins, econazole, econazole nitrate, efavirenz, ellipticine, enalapril, enlimomab, enoximone, epinephrine, epipodophyllotoxin derivatives, epirubicin, epoetinalfa, eposartan, equilenin, equilin, ergocalciferol, ergotamine tartrate, erlotinib, erythromycin, estradiol, estramustine, estriol, estrone, ethacrynic acid, ethambutol, ethinamate, ethionamide, ethopropazine HCl, ethyl-4-aminobenzoate (benzocaine), ethylparaben, ethinylestradiol, etodolac, etomidate, etoposide, etretinate, exemestane, felbamate, felodipine, fenbendazole, fenbuconazole, fenbufen, fenchlorphos, fenclofenac, fenfluramine, fenofibrate, fenoldepam, fenoprofen calcium, fenoxycarb, fenpiclonil, fentanyl, fenticonazole, fexofenadine, filgrastim, finasteride, flecamide acetate, floxuridine, fludarabine, fluconazole, fluconazole, flucytosine, fludioxonil, fludrocortisone, fludrocortisone acetate, flufenamic acid, flunanisone, flunarizine HCl, flunisolide, flunitrazepam, fluocortolone, fluometuron, fluorene, fluorouracil, fluoxetine HCl, fluoxymesterone, flupenthixol decanoate, fluphenthixol decanoate, flurazepam, flurbiprofen, fluticasone propionate, fluvastatin, folic acid, fosenopril, fosphenytoin sodium, frovatriptan, furosemide, fulvestrant, furazolidone, gabapentin, G-BHC (Lindane), gefitinib, gemcitabine, gemfibrozil, gemtuzumab, glafenine, glibenclamide, gliclazide, glimepiride, glipizide, glutethimide, glyburide, Glyceryltrinitrate (nitroglycerin), goserelin acetate, grepafloxacin, griseofulvin, guaifenesin, guanabenz acetate, guanine, halofantrine HCl, haloperidol, hydrochlorothiazide, heptabarbital, heroin, hesperetin, hexachlorobenzene, hexethal, histrelin acetate, hydrocortisone, hydroflumethiazide, hydroxyurea, hyoscyamine, hypoxanthine, ibritumomab, ibuprofen, idarubicin, idobutal, ifosfamide, ihydroequilenin, imatinib mesylate, imipenem, indapamide, indinavir, indomethacin, indoprofen, interferon alfa-2a, interferon alfa-2b, iodamide, iopanoic acid, iprodione, irbesartan, irinotecan, isavuconazole, isocarboxazid, isoconazole, isoguanine, isoniazid, isopropylbarbiturate, isoproturon, isosorbide dinitrate, isosorbide mononitrate, isradipine, itraconazole, itraconazole, itraconazole (Itra), ivermectin, ketoconazole, ketoprofen, ketorolac, khellin, labetalol, lamivudine, lamotrigine, lanatoside C, lanosprazole, L-DOPA, leflunomide, lenalidomide, letrozole, leucovorin, leuprolide acetate, levamisole, levofloxacin, lidocaine, linuron, lisinopril, lomefloxacin, lomustine, loperamide, loratadine, lorazepam, lorefloxacin, lormetazepam, losartan mesylate, lovastatin, lysuride maleate, Maprotiline HCl, mazindol, mebendazole, Meclizine HCl, meclofenamic acid, medazepam, medigoxin, medroxyprogesterone acetate, mefenamic acid, Mefloquine HCl, megestrol acetate, melphalan, mepenzolate bromide, meprobamate, meptazinol, mercaptopurine, mesalazine, mesna, mesoridazine, mestranol, methadone, methaqualone, methocarbamol, methoin, methotrexate, methoxsalen, methsuximide, methyclothiazide, methylphenidate, methylphenobarbitone, methyl-p-hydroxybenzoate, methylprednisolone, methyltestosterone, methyprylon, methysergide maleate, metoclopramide, metolazone, metoprolol, metronidazole, Mianserin HCl, miconazole, midazolam, mifepristone, miglitol, minocycline, minoxidil, mitomycin C, mitotane, mitoxantrone, mofetilmycophenolate, molindone, montelukast, morphine, Moxifloxacin HCl, nabumetone, nadolol, nalbuphine, nalidixic acid, nandrolone, naphthacene, naphthalene, naproxen, naratriptan HCl, natamycin, nelarabine, nelfinavir, nevirapine, nicardipine HCl, nicotin amide, nicotinic acid, nicoumalone, nifedipine, nilutamide, nimodipine, nimorazole, nisoldipine, nitrazepam, nitrofurantoin, nitrofurazone, nizatidine, nofetumomab, norethisterone, norfloxacin, norgestrel, nortriptyline HCl, nystatin, oestradiol, ofloxacin, olanzapine, omeprazole, omoconazole, ondansetron HCl, oprelvekin, ornidazole, oxaliplatin, oxamniquine, oxantelembonate, oxaprozin, oxatomide, oxazepam, oxcarbazepine, oxfendazole, oxiconazole, oxprenolol, oxyphenbutazone, oxyphencyclimine HCl, paclitaxel, palifermin, pamidronate, p-aminosalicylic acid, pantoprazole, paramethadione, paroxetine HCl, pegademase, pegaspargase, pegfilgrastim, pemetrexeddisodium, penicillamine, pentaerythritol tetranitrate, pentazocin, pentazocine, pentobarbital, pentobarbitone, pentostatin, pentoxifylline, perphenazine, perphenazine pimozide, perylene, phenacemide, phenacetin, phenanthrene, phenindione, phenobarbital, phenolbarbitone, phenolphthalein, phenoxybenzamine, phenoxybenzamine HCl, phenoxymethyl penicillin, phensuximide, phenylbutazone, phenytoin, pindolol, pioglitazone, pipobroman, piroxicam, pizotifen maleate, platinum compounds, plicamycin, polyenes, polymyxin B, porfimersodium, posaconazole (Posa), pramipexole, prasterone, pravastatin, praziquantel, prazosin, prazosin HCl, prednisolone, prednisone, primidone, probarbital, probenecid, probucol, procarbazine, prochlorperazine, progesterone, proguanil HCl, promethazine, propofol, propoxur, propranolol, propylparaben, propylthiouracil, prostaglandin, pseudoephedrine, pteridine-2-methyl-thiol, pteridine-2-thiol, pteridine-4-methyl-thiol, pteridine-4-thiol, pteridine-7-methyl-thiol, pteridine-7-thiol, pyrantelembonate, pyrazinamide, pyrene, pyridostigmine, pyrimethamine, quetiapine, quinacrine, quinapril, quinidine, quinidine sulfate, quinine, quininesulfate, rabeprazole sodium, ranitidine HCl, rasburicase, ravuconazole, repaglinide, reposal, reserpine, retinoids, rifabutine, rifampicin, rifapentine, rimexolone, risperidone, ritonavir, rituximab, rizatriptan benzoate, rofecoxib, ropinirole HCl, rosiglitazone, saccharin, salbutamol, salicylamide, salicylic acid, saquinavir, sargramostim, secbutabarbital, secobarbital, sertaconazole, sertindole, sertraline HCl, simvastatin, sirolimus, sorafenib, sparfloxacin, spiramycin, spironolactone, stanolone, stanozolol, stavudine, stilbestrol, streptozocin, strychnine, sulconazole, sulconazole nitrate, sulfacetamide, sulfadiazine, sulfamerazine, sulfamethazine, sulfamethoxazole, sulfanilamide, sulfathiazole, sulindac, sulphabenzamide, sulphacetamide, sulphadiazine, sulphadoxine, sulphafurazole, sulphamerazine, sulpha-methoxazole, sulphapyridine, sulphasalazine, sulphinpyrazone, sulpiride, sulthiame, sumatriptan succinate, sunitinib maleate, tacrine, tacrolimus, talbutal, tamoxifen citrate, tamulosin, targretin, taxanes, tazarotene, telmisartan, temazepam, temozolomide, teniposide, tenoxicam, terazosin, terazosin HCl, terbinafine HCl, terbutaline sulfate, terconazole, terfenadine, testolactone, testosterone, tetracycline, tetrahydrocannabinol, tetroxoprim, thalidomide, thebaine, theobromine, theophylline, thiabendazole, thiamphenicol, thioguanine, thioridazine, thiotepa, thotoin, thymine, tiagabine HCl, tibolone, ticlopidine, tinidazole, tioconazole, tirofiban, tizanidine HCl, tolazamide, tolbutamide, tolcapone, topiramate, topotecan, toremifene, tositumomab, tramadol, trastuzumab, trazodone HCl, tretinoin, triamcinolone, triamterene, triazolam, triazoles, triflupromazine, trimethoprim, trimipramine maleate, triphenylene, troglitazone, tromethamine, tropicamide, trovafloxacin, tybamate, ubidecarenone (coenzyme Q10), undecenoic acid, uracil, uracil mustard, uric acid, valproic acid, valrubicin, valsartan, vancomycin, venlafaxine HCl, vigabatrin, vinbarbital, vinblastine, vincristine, vinorelbine, voriconazole, xanthine, zafirlukast, zidovudine, zileuton, zoledronate, zoledronic acid, zolmitriptan, zolpidem, or zopiclone.

### Polynucleotides

In some embodiments of the pharmaceutical compositions of the present application, the therapeutic agent (or prophylactic agent) assembled with the lipid composition comprises one or more polynucleotides. The present application is not limited in scope to any particular source, sequence, or type of polynucleotide; however, as one of ordinary skill in the art could readily identify related homologs in various other sources of the polynucleotide including nucleic acids from non-human species (e.g., mouse, rat, rabbit, dog, monkey, gibbon, chimp, ape, baboon, cow, pig, horse, sheep, cat and other species). It is contemplated that the polynucleotide used in the present application can comprises a sequence based upon a naturally-occurring sequence. Allowing for the degeneracy of the genetic code, sequences that have at least about 50%, usually at least about 60%, more usually about 70%, most usually about 80%, preferably at least about 90% and most preferably about 95% of nucleotides that are identical to the nucleotide sequence of the naturally-occurring sequence. In another embodiment, the polynucleotide comprises nucleic acid sequence that is a complementary sequence to a naturally occurring sequence, or complementary to 75%, 80%, 85%, 90%, 95% and 100%. Longer polynucleotides encoding 250, 500, 1000, 1212, 1500, 2000, 2500, 3000 or longer are contemplated herein.

In some embodiments, the polynucleotide used herein may be derived from genomic DNA, i.e., cloned directly from the genome of a particular organism. In preferred embodiments, however, the polynucleotide would comprise complementary DNA (cDNA). Also contemplated is a cDNA plus a natural intron or an intron derived from another gene; such engineered molecules are sometime referred to as “mini-genes.” At a minimum, these and other nucleic acids of the present application may be used as molecular weight standards in, for example, gel electrophoresis. The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

In some embodiments, the polynucleotide comprises one or more segments comprising a small interfering ribonucleic acid (siRNA), a short hairpin RNA (shRNA), a micro-ribonucleic acid (miRNA), a primary micro-ribonucleic acid (pri-miRNA), a long non-coding RNA (lncRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), an antisense ribonucleic acid (RNA), a guide ribonucleic acid, deoxyribonucleic acid (DNA), a double stranded deoxyribonucleic acid (dsDNA), a single stranded deoxyribonucleic acid (ssDNA), a single stranded ribonucleic acid (ssRNA), a or double stranded ribonucleic acid (dsRNA). In some embodiments, the polynucleotide encodes at least one of the therapeutic agent (or prophylactic agent) described herein. In some embodiments, the polynucleotide encodes at least one guide polynucleotide, such as guide RNA (gRNA) or guide DNA (gDNA), for complexing with a guide RNA guided nuclease described herein. In some embodiments, the polynucleotide encodes at least one guide polynucleotide guided heterologous nuclease. The nuclease may be an endonuclease. Non-limiting example of the guide polynucleotide guided heterologous endonuclease may be selected from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof, any variant thereof, and any fragment thereof.

Some embodiments of the therapeutic agent (or prophylactic agent) provided herein comprise a heterologous polypeptide comprising an actuator moiety. The actuator moiety can be configured to complex with a target polynucleotide corresponding to a target gene. In some embodiments, administration of the therapeutic agent (or prophylactic agent) results in a modified expression or activity of the target gene. The therapeutic agent (or prophylactic agent) may comprise a heterologous polynucleotide encoding an actuator moiety. The actuator moiety may be configured to complex with a target polynucleotide corresponding to a target gene. The heterologous polynucleotide may encode a guide polynucleotide configured to direct the actuator moiety to the target polynucleotide. The actuator moiety may comprise a heterologous endonuclease or a fragment thereof (e.g., directed by a guide polynucleotide to specifically bind the target polynucleotide). The heterologous endonuclease may be (1) part of a ribonucleoprotein (RNP) and (2) complexed with the guide polynucleotide. The heterologous endonuclease may be part of a clustered regularly interspaced short palindromic repeats (CRISPR)/CRISPR-associated (Cas) protein complex. The heterologous endonuclease may be a clustered regularly interspaced short palindromic repeats (CRISPR)-associated (Cas) endonuclease. The heterologous endonuclease may comprise a deactivated endonuclease. The deactivated endonuclease may be fused to a regulatory moiety. The regulatory moiety may comprise a transcription activator, a transcription repressor, an epigenetic modifier, or a fragment thereof.

In some embodiments, the polynucleotide encodes at least one guide polynucleotide (such as guide RNA (gRNA) or guide DNA (gDNA)) guided heterologous endonuclease. In some embodiments, the polynucleotide encodes at least one guide polynucleotide and at least one heterologous endonuclease, where the guide polynucleotide can be complexed with and guides the at least one heterologous endonuclease to cleave a genetic locus of any one of the genes described herein. In some embodiments, the polynucleotide encodes at least one guide polynucleotide guided heterologous endonuclease such as Cas9, Cas12, Cas13, Cpf1 (or Cas12a), C2C1, C2C2 (or Cas13a), Cas13b, Cas13c, Cas13d, Cas14, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966; any derivative thereof, any variant thereof, or any fragment thereof. In some embodiments, Cas13 can include, but are not limited to, Cas13a, Cas13b, Cas13c, and Cas 13d (e.g., CasRx).

In some embodiments, the heterologous endonuclease comprises a deactivated endonuclease, optionally fused to a regulatory moiety, such as an epigenetic modifier to remodel the epigenome that mediates the expression of the selected genes of interest. In some cases, the epigenetic modifier can include methyltransferase, demethylase, dismutase, an alkylating enzyme, depurinase, oxidase, photolyase, integrase, transposase, recombinase, polymerase, ligase, helicase, glycosylase, acetyltransferase, deacetylase, kinase, phosphatase, ubiquitin-activating enzymes, ubiquitin-conjugating enzymes, ubiquitin ligase, deubiquitinating enzyme, adenylate-forming enzyme, AMPylator, de-AMPylator, SUMOylating enzyme, deSUMOylating enzyme, ribosylase, deribosylase, N-myristoyltransferase, chromotine remodeling enzyme, protease, oxidoreductase, transferase, hydrolase, lyase, isomerase, synthase, synthetase, or demyristoylation enzyme. In some instances, the epigenetic modifier can comprise one or more selected from the group consisting of p300, TET1, LSD1, HDAC1, HDAC8, HDAC4, HDAC11, HDT1, SIRT3, HST2, CobB, SIRT5, SIR2A, SIRT6, NUE, vSET, SUV39H1, DIM5, KYP, SUVR4, Set4, Set1, SETD8, and TgSET8.

In some embodiments, the polynucleotide encodes a guide polynucleotide (such as guide RNA (gRNA) or guide DNA (gDNA)) that is at least partially complementary to the genomic region of a gene, where upon binding of the guide polynucleotide to the gene the guide polynucleotide recruits the guide polynucleotide guided nuclease to cleave and genetically modified the region. Examples of the genes that may be modified by the guide polynucleotide guided nuclease include CFTR, DNAH5, DNAH11, BMPR2, FAH, PAH, IDUA, COL4A3, COL4A4, COL4A5, PKD1, PKD2, PKHD1, SLC3A1, SLC7A9, PAX9, MYO7A, CDH23, USH2A, CLRN1, GJB2, GJB6, RHO, DMPK, DMD, SCN1A, SCN1B, F8, F9, NGLY1, p53, PPT1, TPP1, hERG, PPT1, ATM, or FBN1.

In some embodiments, the polynucleotide comprises or encodes at least one mRNA that, upon expression of the mRNA, restores the function of a defective gene in a subject being treated by the pharmaceutical composition described herein.

In some embodiments, the polynucleotides of the present application comprise at least one chemical modifications of the one or more nucleotides. In some embodiments, the chemical modification increases specificity of the guide polynucleotide (such as guide RNA (gRNA) or guide DNA (gDNA)) binding to a complementary genomic locus (e.g., the genomic locus of any one of the genes described herein). In some embodiments, the at least one chemical modification increases resistance to nuclease digestion, when then polynucleotide is administered to a subject in need thereof. In some embodiments, the at least one chemical modification decreases immunogenicity, when then polynucleotide is administered to a subject in need thereof. In some embodiments, the at least one chemical modification stabilizes scaffold such as a tRNA scaffold. Such chemical modification may have desirable properties, such as enhanced resistance to nuclease digestion or increased binding affinity with a target genomic locus relative to a polynucleotide without the at least one chemical modification.

In some embodiments, the at least one chemical modification comprises modification to sugar moiety. In some embodiments, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain embodiments, sugar substituents at the 2′ position is selected from allyl, amino, azido, thio, O-allyl, O—C₁-C₁₀ alkyl, O—C₁-C₁₀ substituted alkyl; OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl. Examples of sugar substituents at the 5′-position, include, but are not limited to: 5′-methyl (R or S); 5′-vinyl, and 5′-methoxy. In some embodiments, substituted sugars comprise more than one non-bridging sugar substituent, for example, T-F-5′-methyl sugar moieties.

Nucleosides comprising 2′-substituted sugar moieties are referred to as 2′-substituted nucleosides. In some embodiments, a 2′-substituted nucleoside comprises a 2′-substituent group selected from halo, allyl, amino, azido, SH, CN, OCN, CF₃, OCF₃, O, S, or N(Rₘ)-alkyl; O, S, or N(Rₘ)-alkenyl; O, S or N(Rₘ)-alkynyl; O-alkylenyl-O-alkyl, alkynyl, alkaryl, aralkyl, O-alkaryl, O-aralkyl, O(CH₂)₂SCH₃, O(CH₂)₂—O—N(Rₘ)(Rₙ) or O—CH₂—C(═O)—N(Rₘ)(Rₙ), where each Rₘ and Rₙ is, independently, H, an amino protecting group or substituted or unsubstituted C₁-C₁₀ alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some embodiments, a 2′-substituted nucleoside comprises a 2′-substituent group selected from F, NH₂, N₃, OCF₃, O—CH₃, O(CH₂)₃NH₂, CH₂—CH═CH₂, O—CH₂—CH═CH₂, OCH₂CH₂OCH₃, O(CH₂)₂SCH₃, O—(CH₂)₂—O—N(Rₘ)(Rₙ), O(CH₂)₂O(CH₂)₂N(CH₃)₂, and N-substituted acetamide (O—CH₂—C(═O)—N(Rₘ)(Rₙ) where each Rₘ and Rₙ is, independently, H, an amino protecting group or substituted or unsubstituted C₁-C₁₀ alkyl.

In some embodiments, a 2′-substituted nucleoside comprises a sugar moiety comprising a 2′-substituent group selected from F, OCF₃, O—CH₃, OCH₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃.

In some embodiments, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such embodiments, the bicyclic sugar moiety comprises a bridge between the 4′ and the 2′ furanose ring atoms. Examples of such 4′ to 2′ sugar substituents, include, but are not limited to: —[C(Rₐ)(R_{b})]ₙ—, —[C(Rₐ)(R_{b})]ₙ—O—, —C(RₐR_{b})—N(R)—O— or, —C(RₐR_{b})—O—N(R)—; 4′-CH₂-2′, 4′—(CH₂)₂-2′, 4′—(CH₂)—O-2′ (LNA); 4′-(CH₂)—S-2′; 4′—(CH₂)₂—O-2′ (ENA); 4′-CH(CH₃)—O-2′ (cEt) and 4′-CH(CH₂OCH₃)—O-2′, and analogs thereof, 4′-C(CH₃)(CH₃)—O-2′ and analogs thereof, 4′-CH₂—N(OCH₃)-2′ and analogs thereof, 4′-CH₂—O—N(CH₃)-2′; 4′—CH₂—O—N(R)-2′, and 4′-CH₂—N(R)—O-2′-, wherein each R is, independently, H, a protecting group, or C₁-C₁₂ alkyl; 4′-CH₂—N(R)—O-2′, wherein R is H, C₁-C₁₂ alkyl, or a protecting group; 4′-CH₂—C(H)(CH₃)-2′; and 4′-CH₂—C(═CH₂)-2′ and analogs thereof.

In some embodiments, such 4′ to 2′ bridges independently comprise from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ᵣ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH₂—O-2′) BNA, (B) R-D-Methyleneoxy (4′-CH₂—O-2′) BNA (also referred to as locked nucleic acid or LNA), (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) Methoxy(ethyleneoxy) (4′-CH(CH₂OMe)-O-2′) BNA (also referred to as constrained MOE or cMOE).

In some embodiments, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH₂—O-2′) bicyclic nucleosides have been incorporated into antisense polynucleotides that showed antisense activity.

In some embodiments, substituted sugar moieties comprise one or more non-bridging sugar substituent and one or more bridging sugar substituent (e.g., 5′-substituted and 4′-2′ bridged sugars, wherein LNA is substituted with, for example, a 5′-methyl or a 5′-vinyl group).

In some embodiments, modified sugar moieties are sugar surrogates. In some such embodiments, the oxygen atom of the naturally occurring sugar is substituted, e.g., with a sulfur, carbon or nitrogen atom. In some such embodiments, such modified sugar moiety also comprises bridging and/or non-bridging substituents as described above. For example, certain sugar surrogates comprise a 4′-sulfur atom and a substitution at the 2′-position and/or the 5′ position. By way of additional example, carbocyclic bicyclic nucleosides having a 4′-2′ bridge have been described.

In some embodiments, sugar surrogates comprise rings having other than 5-atoms. For example, in some embodiments, a sugar surrogate comprises a six-membered tetrahydropyran. Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA), and fluoro HNA (F-HNA).

Many other bicyclo and tricyclo sugar surrogate ring systems are also known in the art that can be used to modify nucleosides for incorporation into antisense compounds.

Combinations of modifications are also provided without limitation, such as 2′-F-5′-methyl substituted nucleosides and replacement of the ribosyl ring oxygen atom with S and further substitution at the 2′-position or alternatively 5′-substitution of a bicyclic nucleic acid. In some embodiments, a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described.

In some embodiments, the present application provides polynucleotide comprising modified nucleosides. Those modified nucleotides may include modified sugars, modified nucleobases, and/or modified linkages. The specific modifications are selected such that the resulting polynucleotide possesses desirable characteristics. In some embodiments, polynucleotide comprises one or more RNA-like nucleosides. In some embodiments, polynucleotide comprises one or more DNA-like nucleotides.

In some embodiments, nucleosides of the present application comprise one or more unmodified nucleobases. In certain embodiments, nucleosides of the present application comprise one or more modified nucleobases.

In some embodiments, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and O-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (²H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone.

In some embodiments, the present application provides polynucleotide comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. In some embodiments, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein contain one or more asymmetric centers and thus give rise to enantiomers, diastereomers, and other stereoisomeric configurations that may be defined, in terms of absolute stereochemistry, as (R) or (S), a or R such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated polynucleotides of the present application involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol orundecyl residues, aphospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

In some embodiments, the polynucleotides described herein comprise or encode at least one tRNA described herein. In some embodiments, the tRNA expressed from the polynucleotide restores the function of at least one defective tRNA in a subject who is being treated by the pharmaceutical composition described herein. In some embodiments, the at least one tRNA expressed by the polynucleotide described herein may include tRNA that encodes alanine, arginine, asparagine, aspartic acid, cysteine, glutamic acid, glutamine, glycine, histidine, hydroxyproline, isoleucine, leucin, lysine, methionine, phenylaniline, proline, pyroglutamic acid, serine, threonine, tryptophan, tyrosine, or valine. In some embodiments, the at least one tRNA expressed by the polynucleotide described herein may include tRNA that encodes arginine, tryptophan, glutamic acid, glutamine, serine, tyrosine, lysine, leucine, glycine, or cysteine.

### Polypeptides

In some embodiments of the pharmaceutical compositions of the present application, the therapeutic agent (or prophylactic agent) assembled with the lipid composition comprises one or more one or more polypeptides. Some polypeptides may include enzymes such as any one of the nuclease enzymes described herein. For example, the nuclease enzyme may include from CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof, any variant thereof, and any fragment thereof. In some embodiments, the nuclease enzyme may include Cas proteins such as Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

In some embodiments, the Cas protein may be complexed with a guide polynucleotide described herein to be form a CRISPR ribonucleoprotein (RNP).

The nuclease in the compositions described herein may be Cas9 (e.g., from S. pyogenes or S. pneumonia). The CRISPR enzyme can direct cleavage of one or both strands at the location of a target sequence, such as within the target sequence and/or within the complement of the target sequence of any one of the genes described herein.

The CRISPR enzyme may be mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, the present application provides polypeptide containing one or more therapeutic proteins. The therapeutic proteins that may be included in the composition include a wide range of molecules such as cytokines, chemokines, interleukins, interferons, growth factors, coagulation factors, anti-coagulants, blood factors, bone morphogenic proteins, immunoglobulins, and enzymes. Some non-limiting examples of particular therapeutic proteins include Erythropoietin (EPO), Granulocyte colony-stimulating factor (G-CSF), Alpha-galactosidase A, Alpha-L-iduronidase, Thyrotropin a, N-acetylgalactosamine-4-sulfatase (rhASB), Dornase alfa, Tissue plasminogen activator (TPA) Activase, Glucocerebrosidase, Interferon (IF) β-1a, Interferon β-1b, Interferon γ, Interferon α, TNF-α, IL-1 through IL-36, Human growth hormone (rHGH), Human insulin (BHI), Human chorionic gonadotropin a, Darbepoetin a, Follicle-stimulating hormone (FSH), and Factor VIII.

In some embodiments, the polypeptide comprises a peptide sequence that is at least partially identical to any of the therapeutic agent (or prophylactic agent) comprising a peptide sequence. For example, the polypeptide may comprise a peptide sequence that is at least partially identical to an antibody (e.g., a monoclonal antibody) for treating a disease such as cancer.

In some embodiments, the polypeptide comprises a peptide or protein that restores the function of a defective protein in a subject being treated by the pharmaceutical composition described herein.

In some embodiments, the pharmaceutical composition of the present application comprises a plurality of payloads assembled with (e.g., encapsulated within) a lipid composition. The plurality of payloads assembled with the lipid composition may be configured for gene-editing or gene-expression modification. The plurality of payloads assembled with the lipid composition may comprise a polynucleotide encoding an actuator moiety (e.g., comprising a heterologous endonuclease such as Cas) or a polynucleotide encoding the actuator moiety. The plurality of payloads assembled with the lipid composition may further comprise one or more (e.g., one or two) guide polynucleotides. The plurality of payloads assembled with the lipid composition may further comprise one or more donor or template polynucleotides. The plurality of payloads assembled with the lipid composition may comprise a ribonucleoprotein (RNP).

In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no more than (about) 20:1, no more than (about) 15:1, no more than (about) 10:1, or no more than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is no less than (about) 20:1, no less than (about) 15:1, no less than (about) 10:1, or no less than (about) 5:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 10:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 10:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 15:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 5:1 to about 20:1. In some embodiments of the pharmaceutical composition of the present application, the therapeutic agent (or prophylactic agent) is a polynucleotide, and a molar ratio of nitrogen in the lipid composition to phosphate in the polynucleotide (N/P ratio) is from about 15:1 to about 20:1.

In some embodiments of the pharmaceutical composition of the present application, a molar ratio of the therapeutic agent to total lipids of the lipid composition is from about 1:1 to about 1:100. In some embodiments of the pharmaceutical composition of the present application, a molar ratio of the therapeutic agent to total lipids of the lipid composition is from about 1:1 to about 1:50. In some embodiments of the pharmaceutical composition of the present application, a molar ratio of the therapeutic agent to total lipids of the lipid composition is from about 50:1 to about 1:100. In some embodiments of the pharmaceutical composition of the present application, a molar ratio of the therapeutic agent to total lipids of the lipid composition is from about 1:1 to about 1:20. In some embodiments of the pharmaceutical composition of the present application, a molar ratio of the therapeutic agent to total lipids of the lipid composition is from about 20:1 to about 1:50. In some embodiments of the pharmaceutical composition of the present application, a molar ratio of the therapeutic agent to total lipids of the lipid composition is from about 50:1 to about 1:70. In some embodiments of the pharmaceutical composition of the present application, a molar ratio of the therapeutic agent to total lipids of the lipid composition is from about 70:1 to about 1:100. In some embodiments of the pharmaceutical composition of the present application, a molar ratio of the therapeutic agent to total lipids of the lipid composition is no more than (about) 1:1, no more than (about) 1:5, no more than (about) 1:10, no more than (about) 1:15, no more than (about) 1:20, no more than (about) 1:25, no more than (about) 1:30, no more than (about) 1:35, no more than (about) 1:40, no more than (about) 1:45, no more than (about) 1:50, no more than (about) 1:60, no more than (about) 1:70, no more than (about) 1:80, no more than (about) 1:90, or more than (about) 1:100. In some embodiments of the pharmaceutical composition of the present application, a molar ratio of the therapeutic agent to total lipids of the lipid composition is no less than (about) 1:1, no less than (about) 1:5, no less than (about) 1:10, no less than (about) 1:15, no less than (about) 1:20, no less than (about) 1:25, no less than (about) 1:30, no less than (about) 1:35, no less than (about) 1:40, no less than (about) 1:45, no less than (about) 1:50, no less than (about) 1:60, no less than (about) 1:70, no less than (about) 1:80, no less than (about) 1:90, or less than (about) 1:100.

In some embodiments of the pharmaceutical composition of the present application, at least (about) 85%, at least (about) 86%, at least (about) 87%, at least (about) 88%, at least (about) 89%, at least (about) 90%, at least (about) 91%, at least (about) 92%, at least (about) 93%, at least (about) 94%, at least (about) 95%, at least (about) 96%, at least (about) 97%, at least (about) 98%, at least (about) 99%, or (about) 100% of the therapeutic agent is encapsulated in particles of the lipid compositions.

In some embodiments of the pharmaceutical composition of the present application, the lipid composition comprises a plurality of particles characterized by one or more characteristics of the following: (1) a (e.g., average) size of 100 nanometers (nm) or less; (2) a polydispersity index (PDI) of no more than about 0.2; and (3) a zeta potential of −10 millivolts (mV) to 10 mV.

In some embodiments of the pharmaceutical composition of the present application, the lipid composition comprises a plurality of particles with a (e.g., average) size from about 50 nanometers (nm) to about 100 nanometers (nm). In some embodiments of the pharmaceutical composition of the present application, the lipid composition comprises a plurality of particles with a (e.g., average) size from about 70 nanometers (nm) to about 100 nanometers (nm). In some embodiments of the pharmaceutical composition of the present application, the lipid composition comprises a plurality of particles with a (e.g., average) size from about 50 nanometers (nm) to about 80 nanometers (nm). In some embodiments of the pharmaceutical composition of the present application, the lipid composition comprises a plurality of particles with a (e.g., average) size from about 60 nanometers (nm) to about 80 nanometers (nm). In some embodiments of the pharmaceutical composition of the present application, the lipid composition comprises a plurality of particles with a (e.g., average) size of at most about 100 nanometers (nm), at most about 90 nanometers (nm), at most about 85 nanometers (nm), at most about 80 nanometers (nm), at most about 75 nanometers (nm), at most about 70 nanometers (nm), at most about 65 nanometers (nm), at most about 60 nanometers (nm), at most about 55 nanometers (nm), or at most about 50 nanometers (nm). In some embodiments of the pharmaceutical composition of the present application, the lipid composition comprises a plurality of particles with a (e.g., average) size of at least about 100 nanometers (nm), at least about 90 nanometers (nm), at least about 85 nanometers (nm), at least about 80 nanometers (nm), at least about 75 nanometers (nm), at least about 70 nanometers (nm), at least about 65 nanometers (nm), at least about 60 nanometers (nm), at least about 55 nanometers (nm), or at least about 50 nanometers (nm). The (e.g., average) size may be determined by size exclusion chromatography (SEC). The (e.g., average) size may be determined by spectroscopic method(s) or image-based method(s), for example, dynamic light scattering, static light scattering, multi-angle light scattering, laser light scattering, or dynamic image analysis, or a combination thereof.

In some embodiments of the pharmaceutical composition of the present application, the lipid composition comprises a plurality of particles with a polydispersity index (PDI) from about 0.05 to about 0.5. In some embodiments of the pharmaceutical composition of the present application, the lipid composition comprises a plurality of particles with a polydispersity index (PDI) from about 0.1 to about 0.5. In some embodiments of the pharmaceutical composition of the present application, the lipid composition comprises a plurality of particles with a polydispersity index (PDI) from about 0.1 to about 0.3. In some embodiments of the pharmaceutical composition of the present application, the lipid composition comprises a plurality of particles with a polydispersity index (PDI) from about 0.2 to about 0.5. In some embodiments of the pharmaceutical composition of the present application, the lipid composition comprises a plurality of particles with a polydispersity index (PDI) of no more than about 0.5, no more than about 0.4, no more than about 0.3, no more than about 0.2, no more than about 0.1, or no more than about 0.05.

In some embodiments of the pharmaceutical composition of the present application, the lipid composition comprises a plurality of particles with a negative zeta potential of −5 millivolts (mV) or less. In some embodiments of the pharmaceutical composition of the present application, the lipid composition comprises a plurality of particles with a negative zeta potential of −10 millivolts (mV) or less. In some embodiments of the pharmaceutical composition of the present application, the lipid composition comprises a plurality of particles with a negative zeta potential of −15 millivolts (mV) or less. In some embodiments of the pharmaceutical composition of the present application, the lipid composition comprises a plurality of particles with a negative zeta potential of −20 millivolts (mV) or less. In some embodiments of the pharmaceutical composition of the present application, the lipid composition comprises a plurality of particles with a negative zeta potential of −30 millivolts (mV) or less. In some embodiments, the lipid composition comprises a plurality of particles with a zeta potential of 0 millivolts (mV) or less. In some embodiments, the lipid composition comprises a plurality of particles with a zeta potential of 5 millivolts (mV) or less. In some embodiments, the lipid composition comprises a plurality of particles with a zeta potential of 10 millivolts (mV) or less. In some embodiments of the pharmaceutical composition of the present application, the lipid composition comprises a plurality of particles with a negative zeta potential of 15 millivolts (mV) or less. In some embodiments of the pharmaceutical composition of the present application, the lipid composition comprises a plurality of particles with a negative zeta potential of 20 millivolts (mV) or less.

In some embodiments of the pharmaceutical composition of the present application, the lipid composition comprises a plurality of particles with a negative zeta potential of −5 millivolts (mV) or more. In some embodiments of the pharmaceutical composition of the present application, the lipid composition comprises a plurality of particles with a negative zeta potential of −10 millivolts (mV) or more In some embodiments of the pharmaceutical composition of the present application, the lipid composition comprises a plurality of particles with a negative zeta potential of −15 millivolts (mV) or more. In some embodiments of the pharmaceutical composition of the present application, the lipid composition comprises a plurality of particles with a negative zeta potential of −20 millivolts (mV) or more. In some embodiments of the pharmaceutical composition of the present application, the lipid composition comprises a plurality of particles with a negative zeta potential of −30 millivolts (mV) or more. In some embodiments, the lipid composition comprises a plurality of particles with a zeta potential of 0 millivolts (mV) or more. In some embodiments, the lipid composition comprises a plurality of particles with a zeta potential of 5 millivolts (mV) or more. In some embodiments, the lipid composition comprises a plurality of particles with a zeta potential of 10 millivolts (mV) or more. In some embodiments of the pharmaceutical composition of the present application, the lipid composition comprises a plurality of particles with a zeta potential of 15 millivolts (mV) or more. In some embodiments of the pharmaceutical composition of the present application, the lipid composition comprises a plurality of particles with a zeta potential of 20 millivolts (mV) or more.

In some embodiments of the pharmaceutical composition of the present application, the lipid composition has an apparent ionization constant (pKa) outside a range of 6 to 7. In some embodiments of the pharmaceutical composition of the present application, the lipid composition has an apparent pKa of about 8 or higher, about 9 or higher, about 10 or higher, about 11 or higher, about 12 or higher, or about 13 or higher. In some embodiments of the pharmaceutical composition of the present application, the lipid composition has an apparent pKa of about 8 to about 13. In some embodiments of the pharmaceutical composition of the present application, the lipid composition has an apparent pKa of about 8 to about 10. In some embodiments of the pharmaceutical composition of the present application, the lipid composition has an apparent pKa of about 9 to about 11. In some embodiments of the pharmaceutical composition of the present application, the lipid composition has an apparent pKa of about 10 to about 13. In some embodiments of the pharmaceutical composition of the present application, the lipid composition has an apparent pKa of about 8 to about 12. In some embodiments of the pharmaceutical composition of the present application, the lipid composition has an apparent pKa of about 10 to about 12.

In some embodiments of the composition, the SORT lipid in the composition achieves about least 1.1-fold greater therapeutic effect in a spleen cell compared to that achieved in a lung cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, therapeutic effect in a spleen cell compared to that achieved in a lung cell. In some embodiments of the composition, the SORT lipid in the composition achieves about least 1.1-fold greater therapeutic effect in a spleen cell compared to that achieved in a liver cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater therapeutic effect in a spleen cell compared to that achieved in a liver cell. In some embodiments of the composition, the SORT lipid in the composition achieves about least 1.1-fold greater therapeutic effect in a lung cell compared to that achieved in a liver cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater therapeutic effect in a lung cell compared to that achieved in a liver cell.

In some embodiments of the composition, the SORT lipid in the composition achieves about least 1.1-fold greater therapeutic effect in a lung cell compared to that achieved in a spleen cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater therapeutic effect in a lung cell compared to that achieved in a spleen cell. In some embodiments of the composition, the SORT lipid in the composition achieves about least 1.1-fold greater therapeutic effect in a spleen cell compared to that achieved in a liver cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater therapeutic effect in a spleen cell compared to that achieved in a liver cell. In some embodiments of the composition, the SORT lipid in the composition achieves about least 1.1-fold greater therapeutic effect in a lung cell compared to that achieved in a liver cell. In some embodiments, the SORT lipid in the composition achieves about 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater therapeutic effect in a lung cell compared to that achieved in a liver cell.

### Methods

In some embodiments of the method, the pharmaceutical composition of the present application can be administrated through any suitable routes including, for example, oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments of the method, the pharmaceutical composition of the present application can be administered in a local rather than systemic manner, for example, via injection of the pharmaceutical composition directly into a targeted tissue, preferably in a sustained release formulation. Local delivery can be affected in various ways, depending on the tissue to be targeted.

In some embodiments, the composition of the present application can be injected into the site of injury, disease manifestation, or pain, for example. In some embodiments, the composition of the present application can be provided in lozenges for oral, tracheal, or esophageal application. In some embodiments, the composition of the present application can be supplied in liquid, tablet or capsule form for administration to the stomach or intestines. In some embodiments, the composition of the present application can be supplied in suppository form for rectal or vaginal application. In some embodiments, the composition of the present application can even be delivered to the eye by use of creams, drops, or even injection.

In some embodiments, provided herein is a method for potent delivery to a cell of a subject comprising administrating to the subject the pharmaceutical composition as described in the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (iii) a selective organ targeting (SORT) lipid separate from the ionizable cationic lipid. The lipid composition may further comprise a phospholipid.

In some embodiments of any method described herein, the method of delivery of a therapeutic agent to a spleen cell comprising (e.g., systemically) administering a composition described herein, thereby providing an effective amount or activity of said therapeutic agent in said spleen cell of said subject that is at least 1.1-fold greater than a corresponding amount or activity of said therapeutic agent achieved in a lung cell of said subject. In some embodiments, the effective amount or activity of said therapeutic agent in said spleen cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, at least 20-fold greater, at least 30-fold greater, at least 40-fold greater, at least 50-fold greater, at least 75-fold greater, at least 100-fold greater, at least 200-fold greater, or at least 300-fold greater, than a corresponding amount or activity of said therapeutic agent achieved in a lung cell of said subject.

In some embodiments of the method of delivery of the therapeutic agent to the spleen cell, the method provides an effective amount or activity of said therapeutic agent in said spleen cell of said subject that is at least 1.1-fold greater than a corresponding amount or activity of said therapeutic agent achieved in a liver cell of said subject. In some embodiments, the effective amount or activity of said therapeutic agent in said spleen cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of said therapeutic agent achieved in a liver cell of said subject.

In some embodiments of the method of delivery of the therapeutic agent to the spleen cell, the method provides an effective amount or activity of said therapeutic agent in said lung cell of said subject that is at least 1.1-fold greater than a corresponding amount or activity of said therapeutic agent achieved in a liver cell of said subject. In some embodiments, the effective amount or activity of said therapeutic agent in said lung cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of said therapeutic agent achieved in a liver cell of said subject.

In some embodiments any method described herein, the method of delivery of a therapeutic agent to a lung cell comprising (e.g., systemically) administering a composition described herein, thereby providing an effective amount or activity of said therapeutic agent in said lung cell of said subject that is at least 1.1-fold greater than a corresponding amount or activity of said therapeutic agent achieved in a spleen cell of said subject. In some embodiments, the effective amount or activity of said therapeutic agent in said lung cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of said therapeutic agent achieved in a spleen cell of said subject.

In some embodiments of the method of delivery of the therapeutic agent to the lung cell, the method provides an effective amount or activity of said therapeutic agent in said lung cell of said subject that is at least 1.1-fold greater than a corresponding amount or activity of said therapeutic agent achieved in a liver cell of said subject. In some embodiments, the effective amount or activity of said therapeutic agent in said lung cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of said therapeutic agent achieved in a liver cell of said subject.

In some embodiments of the method of delivery of the therapeutic agent to the lung cell, the method provides an effective amount or activity of said therapeutic agent in said spleen cell of said subject that is at least 1.1-fold greater than a corresponding amount or activity of said therapeutic agent achieved in a liver cell of said subject. In some embodiments, the effective amount or activity of said therapeutic agent in said spleen cell is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 7-fold greater, at least 8-fold greater, at least 9-fold greater, at least 10-fold greater, at least 15-fold greater, at least 18-fold greater, or at least 20-fold greater than a corresponding amount or activity of said therapeutic agent achieved in a liver cell of said subject.

In some embodiments, the delivery of the therapeutic to a cell may alter the genome, transcriptome, or expression levels. The cell may be allowed to, or able to, propagate and the alteration may be passed on to the cells generated from the cell that the therapeutic was delivered to. In this manner, the therapeutic effect may be propagated to a larger number of cells. The alteration to the genome, transcriptome or expression level may also persist in a given cell.

### Dosing Level

In another aspect, provided is high-potency dosage form of a therapeutic agent (or prophylactic agent) formulated with a selective organ targeting (SORT) lipid, the dosage form comprising a therapeutic agent (or prophylactic agent) assembled with a lipid composition as described herein. In some embodiments, the lipid composition comprises: (i) an ionizable cationic lipid; and (ii) a selective organ targeting (SORT) lipid separate from the ionizable cationic lipid. The lipid composition may further comprise a phospholipid.

In some embodiments, the therapeutic agent is present in the dosage form at a dose of about 2.0, 1.5, 1.0, 0.5, 0.2, or 0.1 milligram per kilogram (mg/kg, or mpk) body weight, or of a range between (inclusive) any two of the foregoing values.

In some embodiments, the therapeutic agent is present in the dosage form at a dose of no more than about 2 milligram per kilogram (mg/kg, or mpk) body weight. In some embodiments, the therapeutic agent is present in the dosage form at a dose of no more than about 1 milligram per kilogram (mg/kg, or mpk) body weight. In some embodiments, the therapeutic agent is present in the dosage form at a dose of no more than about 0.5 milligram per kilogram (mg/kg, or mpk) body weight. In some embodiments, the therapeutic agent is present in the dosage form at a dose of no more than about 0.2 milligram per kilogram (mg/kg, or mpk) body weight. In some embodiments, the therapeutic agent is present in the dosage form at a dose of no more than about 0.1 milligram per kilogram (mg/kg, or mpk) body weight. In some embodiments, the therapeutic agent is present in the dosage form at a concentration of no more than about 5 milligram per milliliter (mg/mL).

In some embodiments, the therapeutic agent is present in the dosage form at a concentration of about 5, 4, 3, 2, 1, 0.5, 0.2, or 0.1 milligram per milliliter (mg/mL), or of a range between (inclusive) any two of the foregoing values.

In some embodiments, the therapeutic agent is present in the dosage form at a concentration of no more than about 5 milligram per milliliter (mg/mL). In some embodiments, the therapeutic agent is present in the dosage form at a concentration of no more than about 2 milligram per milliliter (mg/mL). In some embodiments, the therapeutic agent is present in the dosage form at a concentration of no more than about 1 milligram per milliliter (mg/mL). In some embodiments, the therapeutic agent is present in the dosage form at a concentration of no more than about 0.5 milligram per milliliter (mg/mL). In some embodiments, the therapeutic agent is present in the dosage form at a concentration of no more than about 0.1 milligram per milliliter (mg/mL).

Any suitable dosage form can be prepared for delivery, for example, via oral, rectal, vaginal, transmucosal, pulmonary including intratracheal or inhaled, or intestinal administration; parenteral delivery, including intramuscular, subcutaneous, intramedullary injections, as well as intrathecal, direct intraventricular, intravenous, intraperitoneal, intranasal, or intraocular injections.

In some embodiments, the dosage form can be administered in a local rather than systemic manner, for example, via injection of the pharmaceutical composition directly into a targeted tissue, preferably in a sustained release formulation. Local delivery can be affected in various ways, depending on the tissue to be targeted.

In some embodiments, the dosage form is an inhaled aerosol containing the composition of the present for nasal, tracheal, or bronchial delivery. In some embodiments, the dosage form can be provided in lozenges for oral, tracheal, or esophageal application. In some embodiments, the dosage form can be supplied in liquid, tablet or capsule form for administration to the stomach or intestines. In some embodiments, the dosage form can be supplied in suppository form for rectal or vaginal application. In some embodiments, the dosage form can be can even be delivered to the eye by use of creams, drops, or even injection.

In some embodiments, the administration of a dose of the therapeutic agent may be repeated.

### Subject

Any subject in need thereof can be treated with the method of the present application. In some embodiments, the subject has been determined to likely respond to the therapeutic agent. For example, the subject may have, is suffering from, or suspected of having a disease or condition. The therapeutic or prophylactic agent(s) as described elsewhere herein may be effective for providing a therapeutic effect for the subject by a variety of mechanisms, for example, via gene therapy (e.g., requiring repeated administration), altered (e.g., increased) protein production, (e.g., in vivo) chimeric antigen receptor (CAR) T-cell generation, immuno-oncology, vaccine-based approach, reactivation of tumor suppressors, or other mechanisms.

In some embodiments, the subject has been determined to have a (e.g., missense or nonsense) mutation in a target gene. In some embodiments, the mutation in the target gene is associated with a genetic disease or disorder.

In some embodiments, the subject has been determined to exhibit an aberrant expression or activity of a protein or polynucleotide that corresponds to a target gene. In some embodiments, the aberrant expression or activity of the protein or polynucleotide is associated with a genetic disease or disorder

In some embodiments, the subject is selected from the group consisting of mouse, rat, monkey, and human. In some embodiments, the subject is a human.

In another aspect, provided herein is a method for potent delivery of a therapeutic agent (or prophylactic agent) to a cell comprising contacting the cell with the pharmaceutical composition of the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (iii) a selective organ targeting (SORT) lipid separate from the ionizable cationic lipid. The lipid composition may further comprise a phospholipid.

In some embodiments of the method, the cell is isolated from the subject. In some embodiments of the method, the cell is a cell line.

In another aspect, provided herein is a method for targeted delivery of a therapeutic agent (or prophylactic agent) to a cell type comprising contacting the cell with the pharmaceutical composition of the present application. In some embodiments of the method, the pharmaceutical composition comprises a therapeutic agent (or prophylactic agent) assembled with a lipid composition as described in the present application, wherein the lipid composition comprises (i) an ionizable cationic lipid; and (ii) a selective organ targeting (SORT) lipid separate from the ionizable cationic lipid. The lipid composition may further comprise a phospholipid.

In some embodiments, the contacting is ex vivo. In some embodiments, the contacting is in vitro. In some embodiments, the contacting is in vivo. In some embodiments, the contacting comprises administering to a subject the composition comprising the therapeutic agent assembled with the lipid composition.

The following are examples of compositions and evaluations of compositions of the disclosure. It is understood that various other embodiments may be practiced, given the general description provided above.

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A composition formulated for systemic (e.g., intravenous) administration, the composition comprising a therapeutic agent assembled with a lipid composition that comprises:
(i) an ionizable cationic lipid;
(ii) a polymer-conjugated lipid; and
(iii) a selective organ targeting (SORT) lipid has the structure of Formula (IA), or a pharmaceutically acceptable salt, stereoisomer, tautomer thereof:

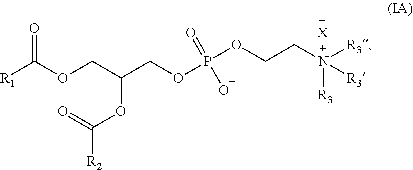

wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

Embodiment 2. The composition of Embodiment 1, wherein the SORT lipid having the structure of Formula (IA) is selected from the group consisting of:

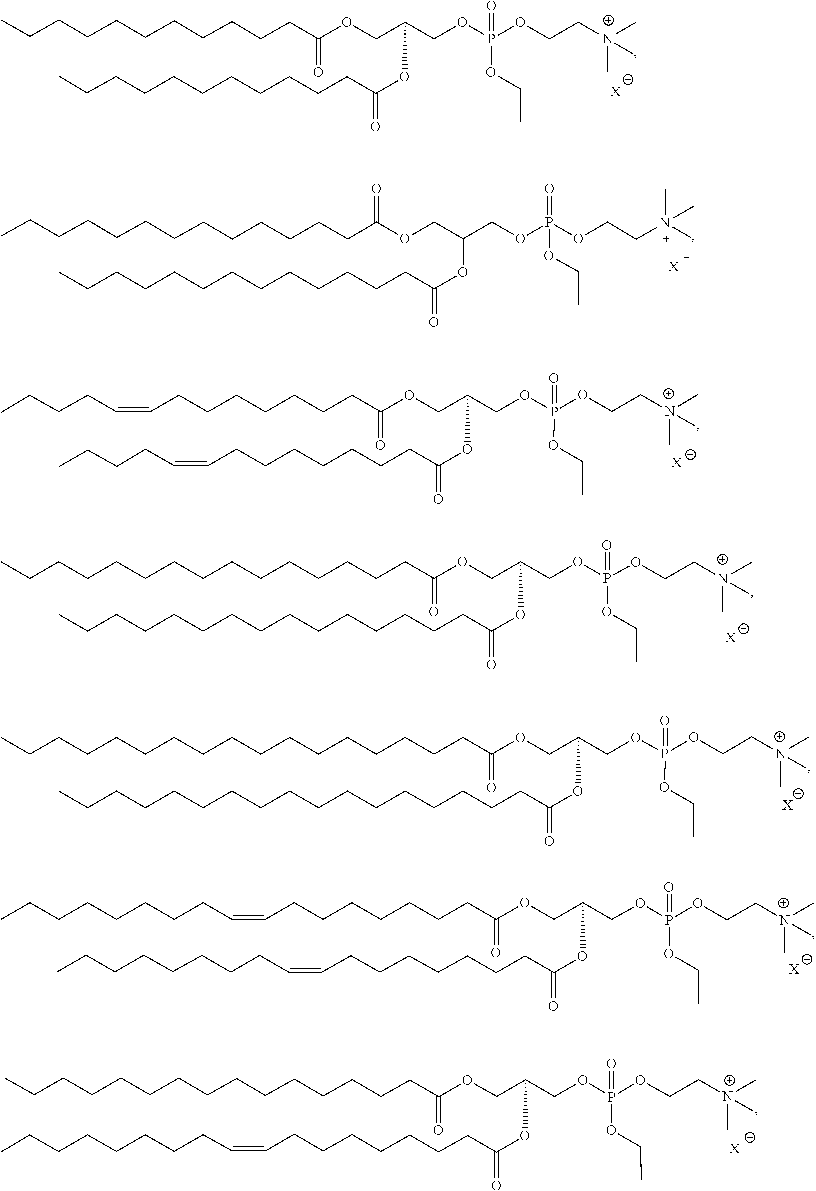

Embodiment 3. A composition formulated for systemic (e.g., intravenous) administration, the composition comprising a therapeutic agent assembled with a lipid composition that comprises:
(i) an ionizable cationic lipid;
(ii) a polymer-conjugated lipid; and
(iii) a selective organ targeting (SORT) lipid has the structure of Formula (S-III), or a pharmaceutically acceptable salt, stereoisomer, tautomer thereof:

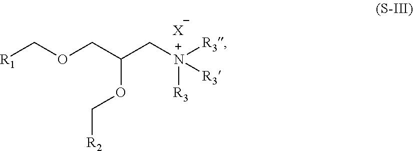

wherein:
   R₁ and R₂ are each independently alkyl_{(C8-C24)}, alkenyl_{(C8-C24)}, or a substituted version of either group;
   R₃, R₃′, and R₃″ are each independently alkyl_{(C≤6)} or substituted alkyl_{(C≤6)}; and
X⁻ is a monovalent anion.

Embodiment 4. The composition of Embodiment 3, wherein the SORT lipid having the structure of Formula (S-III) is

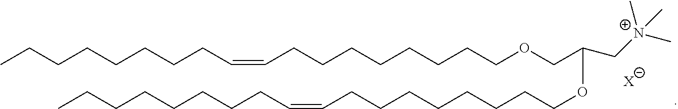

Embodiment 5. The composition of any one of Embodiments 1-4, wherein the ionizable cationic lipid is a dendrimer or dendron of a generation (g) having a structural formula:

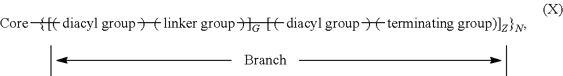

or a pharmaceutically acceptable salt thereof, wherein:
(a) the core comprises a structural formula (X_{Core}):

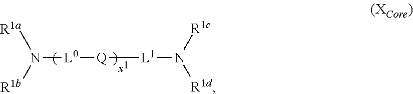

wherein:
   Q is independently at each occurrence a covalent bond, —O—, —S—, —NR²—, or —CR^{3a}R^{3b}—;
   R² is independently at each occurrence R^{1g} or -L²-NR^{1e}R^{1f};
   R^{3a} and R^{3b} are each independently at each occurrence hydrogen or an optionally substituted (e.g., C₁-C₆, such as C₁-C₃) alkyl;
   R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
   L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
   alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
   x¹ is 0, 1, 2, 3, 4, 5, or 6; and
(b) each branch of the plurality (N) of branches independently comprises a structural formula (X_{Branch}):

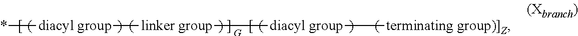

wherein:
   * indicates a point of attachment of the branch to the core;
   g is 1, 2, 3, or 4;
   Z=2^{(g-1)};
   G=0, when g=1; or G=Σᵢ₌₀^{i=g-2}, when g≠1;
(c) each diacyl group independently comprises a structural formula

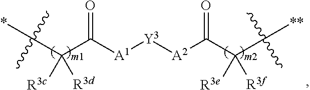

wherein:
   * indicates a point of attachment of the diacyl group at the proximal end thereof,
   ** indicates a point of attachment of the diacyl group at the distal end thereof,
   Y³ is independently at each occurrence an optionally substituted (e.g., C₁-C₁₂); alkylene, an optionally substituted (e.g., C₁-C₁₂) alkenylene, or an optionally substituted (e.g., C₁-C₁₂) arenylene;
   A¹ and A² are each independently at each occurrence —O—, —S—, or —NR⁴—, wherein:
      R⁴ is hydrogen or optionally substituted (e.g., C₁-C₆) alkyl;
   m¹ and m² are each independently at each occurrence 1, 2, or 3; and
   R^{3c}, R^{3d}, R^{3e}, and R^{3f} are each independently at each occurrence hydrogen or an optionally substituted (e.g., C₁-C₈) alkyl; and
(d) each linker group independently comprises a structural formula

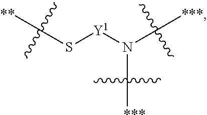

wherein:
   ** indicates a point of attachment of the linker to a proximal diacyl group;
   *** indicates a point of attachment of the linker to a distal diacyl group; and
   Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

Embodiment 6. The composition of Embodiment 5, wherein x¹ is 0, 1, 2, or 3.

Embodiment 7. The composition of Embodiment 5 or 6, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl

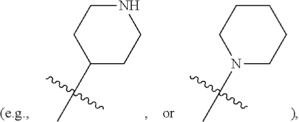 [SMILES: *C1CCNCC1 ; *N1CCCCC1]

N—(C₁-C₃ alkyl)-piperidinyl

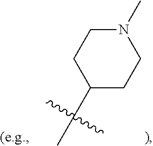 [SMILES: *C1CCN(C)CC1]

piperazinyl

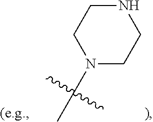 [SMILES: *N1CCNCC1]

N—(C₁-C₃ alkyl)-piperadizinyl

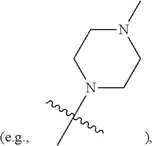 [SMILES: *N1CCN(C)CC1]

morpholinyl

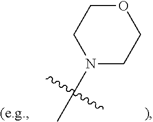 [SMILES: *N1CCOCC1]

### N-pyrrolidinyl

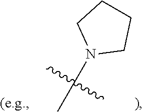 [SMILES: *N1CCCC1]

pyrrolidinyl

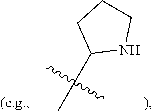 [SMILES: *C1CCCN1]

or N—(C₁-C₃ alkyl)-pyrrolidinyl

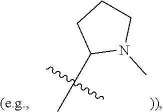 [SMILES: *C1CCCN1C]

(e.g., C₆-C₁₀) aryl, and C₃-C₅ heteroaryl (e.g., imidazolyl

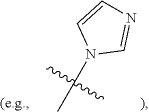 [SMILES: *n1ccnc1]

or pyridinyl

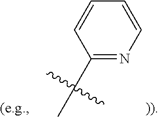 [SMILES: *c1ccccn1]

Embodiment 8. The method of Embodiment 7, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

Embodiment 9. The composition of any one of Embodiments 5-8, wherein R^{3a} and R^{3b} are each independently at each occurrence hydrogen.

Embodiment 10. The composition of any one of Embodiments 5-9, wherein the plurality (N) of branches comprises at least 3 (e.g., at least 4, or at least 5) branches.

Embodiment 11. The composition of any one of Embodiments 5-10, wherein g=1; G=0; and Z=1.

Embodiment 12. The composition of Embodiment 11, wherein each branch of the plurality of branches comprises a structural formula

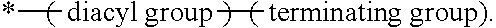

Embodiment 13. The composition of any one of Embodiments 5-10, wherein g=2; G=1; and Z=2.

Embodiment 14. The composition of Embodiment 13, wherein each branch of the plurality of branches comprises a structural formula

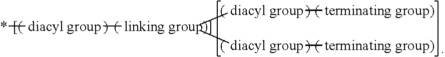

Embodiment 15. The composition of any one of Embodiments 5-14, wherein the core comprises a structural formula:

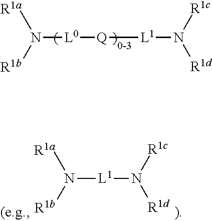

Embodiment 16. The composition of any one of Embodiments 5-14, wherein the core comprises a structural formula:

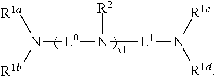

Embodiment 17. The composition of Embodiment 16, wherein the core comprises a structural formula:

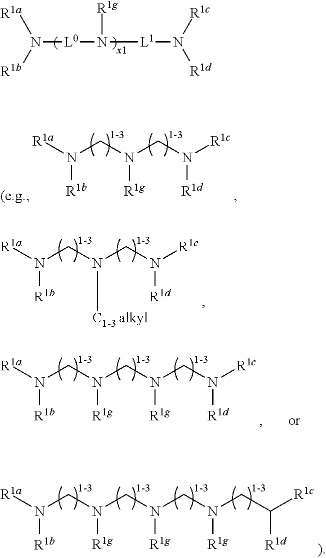

Embodiment 18. The composition of Embodiment 16, wherein the core comprises a structural formula:

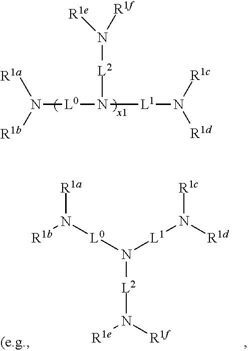

such as

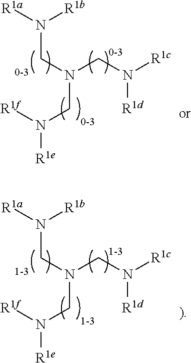

or

Embodiment 19. The composition of any one of Embodiments 5-14, wherein the core comprises a structural formula:

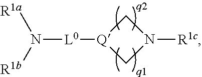

wherein Q′ is —NR²— or —CR^{3a}R^{3b}—; q¹ and q² are each independently 1 or 2.

Embodiment 20. The composition of Embodiment 19, wherein the core comprises a structural formula:

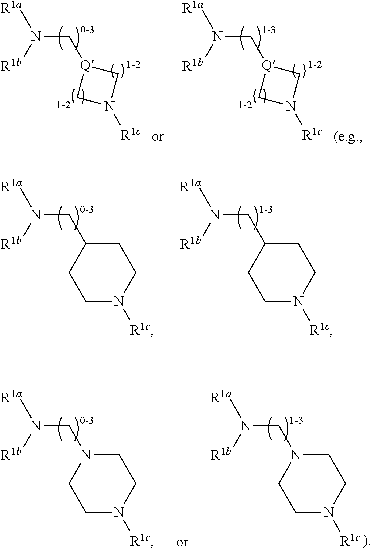

Embodiment 21. The composition of any one of Embodiments 5-14, wherein the core comprises a structural formula

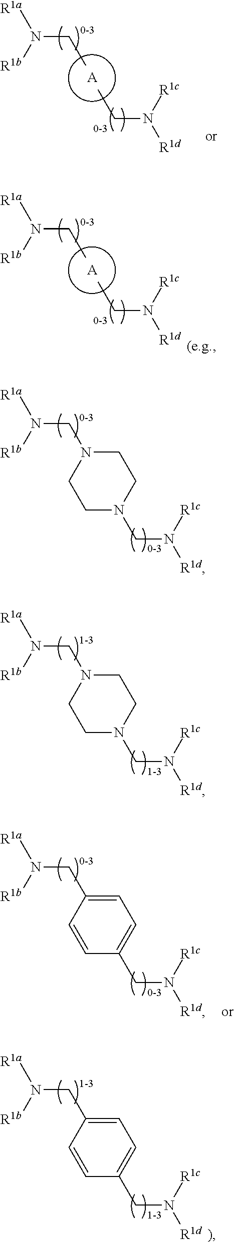

wherein ring A is an optionally substituted aryl or an optionally substituted (e.g., C₃-C₁₂, such as C₃-C₅) heteroaryl.

Embodiment 22. The composition of any one of Embodiments 5-14, wherein the core comprises has a structural formula

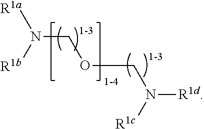

Embodiment 23. The composition of any one of Embodiments 5-14, wherein the core is selected from those set forth in Table 1 or a subset thereof.

Embodiment 24. The composition of any one of Embodiments 5-14, wherein the core comprises a structural formula selected from the group consisting of:

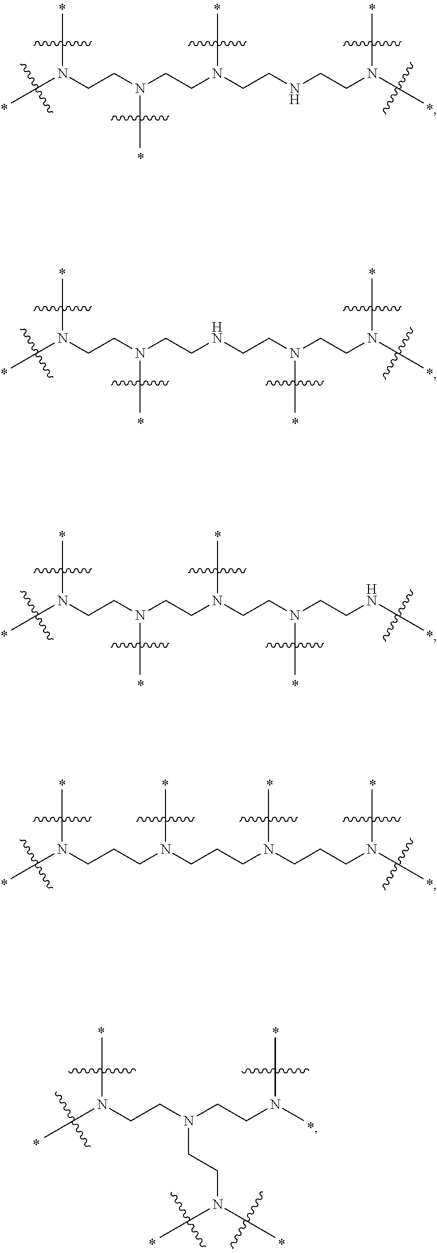 [SMILES: *N(*)CCNCCN(*)CCN(*)CCN(*)* ; *N(*)CCN(*)CCNCCN(*)CCN(*)* ; *NCCN(*)CCN(*)CCN(*)CCN(*)* ; *N(*)CCCN(*)CCCN(*)CCCN(*)* ; *N(*)CCN(CCN(*)*)CCN(*)C]

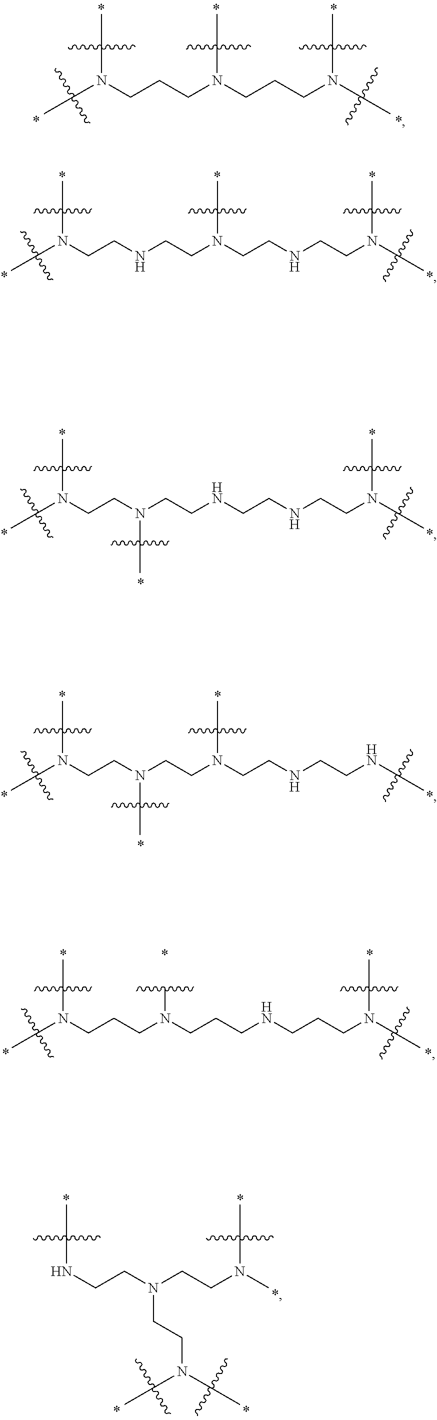 [SMILES: *N(*)CCCN(*)CCCN(*)* ; *N(*)CCNCCN(*)CCNCCN(*)* ; *N(*)CCNCCNCCN(*)CCN(*)* ; *NCCNCCN(*)CCN(*)CCN(*)* ; *N(*)CCCNCCCN(*)CCCN(*)* ; *NCCN(CCN(*)*)CCN(*)C]

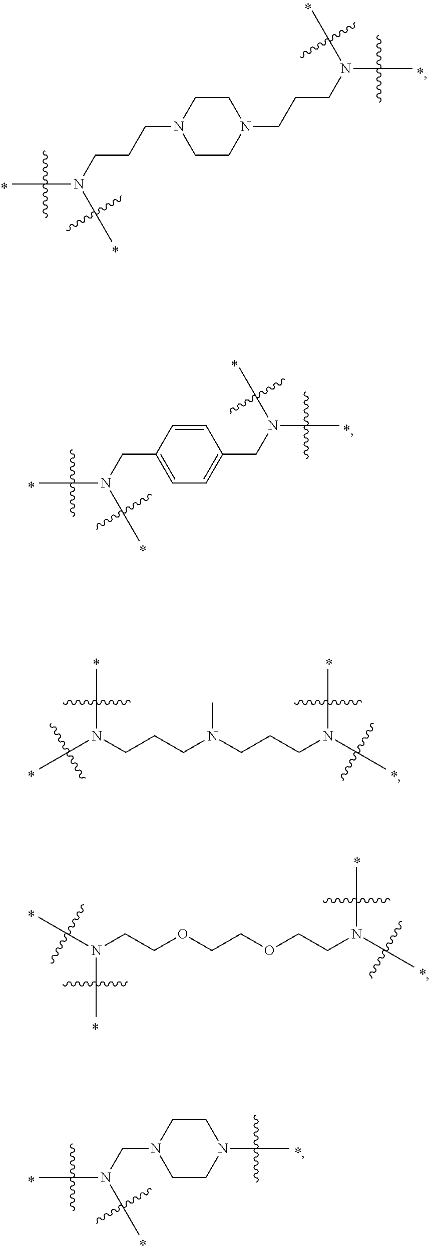 [SMILES: *N(*)CCCN1CCN(CCCN(*)*)CC1 ; *N(*)Cc1ccc(CN(*)*)cc1 ; *N(*)CCCN(C)CCCN(*)* ; *N(*)CCOCCOCCN(*)* ; *N(*)CN1CCN(*)CC1]

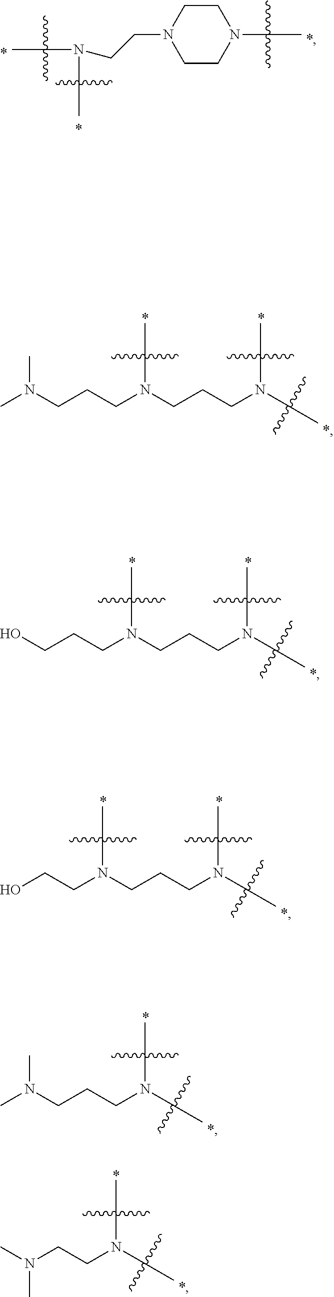

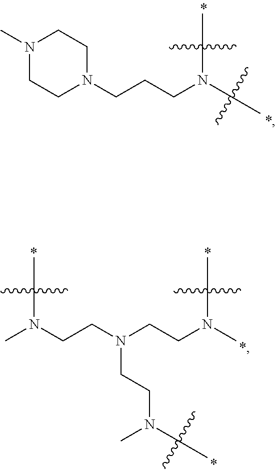

and pharmaceutically acceptable salts thereof, wherein * indicates a point of attachment of the core to a branch of the plurality of branches.

Embodiment 25. The composition of any one of Embodiments 5-14, wherein the core comprises a structural formula selected from the group consisting of:

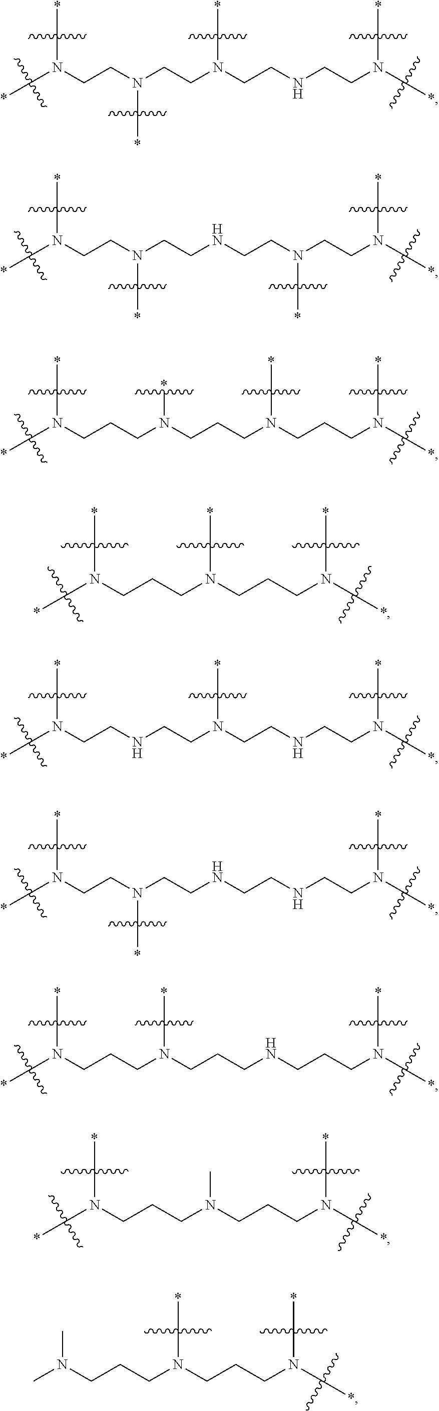

and pharmaceutically acceptable salts thereof, wherein * indicates a point of attachment of the core to a branch of the plurality of branches.

Embodiment 26. The composition of any one of Embodiments 5-14, wherein the core has the structure

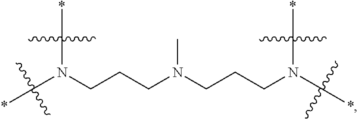

wherein * indicates a point of attachment of the core to a branch of the plurality of branches or H.

Embodiment 27. The composition of Embodiment 26, wherein at least 2 branches are attached to the core.

Embodiment 28. The composition of Embodiment 26, wherein at least 3 branches are attached to the core.

Embodiment 29. The composition of Embodiment 26, wherein at least 4 branches are attached to the core.

Embodiment 30. The composition of any one of Embodiments 5-14, wherein the core has the structure

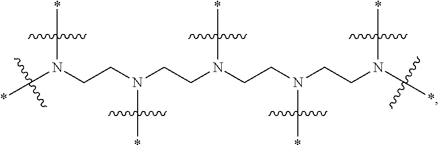

wherein * indicates a point of attachment of the core to a branch of the plurality of branches or H.

Embodiment 31. The composition of Embodiment 30, wherein at least 4 branches are attached to the core.

Embodiment 32. The composition of Embodiment 30, wherein at least 5 branches are attached to the core.

Embodiment 33. The composition of Embodiment 30, wherein at least 6 branches are attached to the core.

Embodiment 34. The composition of any one of Embodiments 5-33, wherein A¹ is —O— or —NH—.

Embodiment 35. The composition of Embodiment 34, wherein A¹ is —O—.

Embodiment 36. The composition of any one of Embodiments 5-35, wherein A² is —O— or —NH—.

Embodiment 37. The composition of any Embodiment 36, wherein A² is —O—.

Embodiment 38. The composition of any one of Embodiments 5-37, wherein Y³ is C₁-C₁₂ (e.g., C₁-C₆, such as C₁-C₃) alkylene.

Embodiment 39. The composition of any one of Embodiments 5-38, wherein the diacyl group independently at each occurrence comprises a structural formula

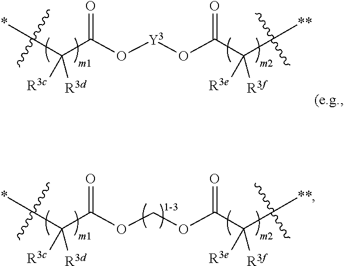

such as

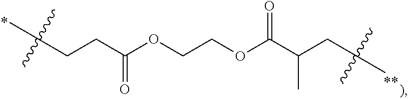 [SMILES: *CCC(=O)OCCOC(=O)C(C)C*]

optionally wherein R^{3c}, R^{3d}, R^{3e}, and R^{3f} are each independently at each occurrence hydrogen or C₁-C₃ alkyl.

Embodiment 40. The composition of any one of Embodiments 5-39, wherein L⁰, L¹, and L² are each independently at each occurrence selected from a covalent bond, C₁-C₆ alkylene (e.g., C₁-C₃ alkylene), C₂-C₁₂ (e.g., C₂-C₈) alkyleneoxide (e.g., oligo(ethyleneoxide), such as —(CH₂CH₂O)₁₋₄—(CH₂CH₂)—), [(C₁-C₄) alkylene]-[(C₄-C₆) heterocycloalkyl]-[(C₁-C₄) alkylene]

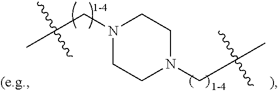

and [(C₁-C₄) alkylene]-phenylene-[(C₁-C₄) alkylene]

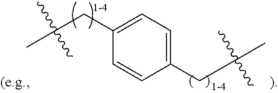

Embodiment 41. The composition of Embodiment 40, wherein L⁰, L¹, and L² are each independently at each occurrence selected from C₁-C₆ alkylene (e.g., C₁-C₃ alkylene), —(C₁-C₃ alkylene-O)₁₋₄—(C₁-C₃ alkylene), —(C₁-C₃ alkylene)-phenylene-(C₁-C₃ alkylene)-, and —(C₁-C₃ alkylene)-piperazinyl-(C₁-C₃ alkylene)-.

Embodiment 42. The composition of Embodiment 40, wherein L⁰, L¹, and L² are each independently at each occurrence C₁-C₆ alkylene (e.g., C₁-C₃ alkylene).

Embodiment 43. The composition of Embodiment 40, wherein L⁰, L¹, and L² are each independently at each occurrence C₂-C₁₂ (e.g., C₂-C₈) alkyleneoxide (e.g., —(C₁-C₃ alkylene-O)₁₋₄—(C₁-C₃ alkylene)).

Embodiment 44. The composition of Embodiment 40, wherein L⁰, L¹, and L² are each independently at each occurrence selected from [(C₁-C₄) alkylene]-[(C₄-C₆) heterocycloalkyl]-[(C₁-C₄) alkylene] (e.g., —(C₁-C₃ alkylene)-phenylene-(C₁-C₃ alkylene)-) and [(C₁-C₄) alkylene]-[(C₄-C₆) heterocycloalkyl]-[(C₁-C₄) alkylene] (e.g., —(C₁-C₃ alkylene)-piperazinyl-(C₁-C₃ alkylene)-).

Embodiment 45. The composition of any one of Embodiments 5-44, wherein each terminating group is independently C₁-C₁₈ (e.g., C₄-C₁₈) alkenylthiol or C₁-C₁₈ (e.g., C₄-C₁₈) alkylthiol, wherein the alkyl or alkenyl moiety is optionally substituted with one or more substituents each independently selected from halogen, C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

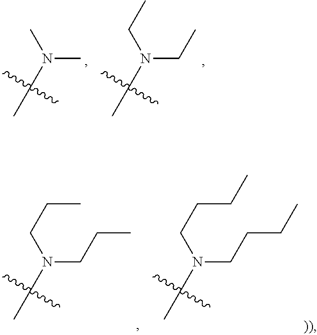

C₄-C₆ N-heterocycloalkyl (e.g., N-pyrrolidinyl

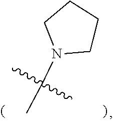 [SMILES: *N1CCCC1]

### N-piperidinyl

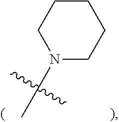 [SMILES: *N1CCCCC1]

### N-azepanyl

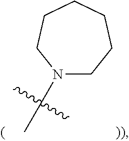 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

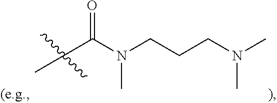 [SMILES: *C(=O)N(C)CCCN(C)C]

—C(O)N(C₁-C₃ alkyl)-(C₁-C₆ alkylene)-(C₄-C₆ N-heterocycloalkyl)

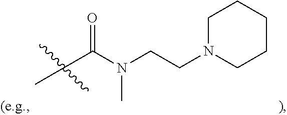 [SMILES: *C(=O)N(C)CCN1CCCCC1]

—C(O)—(C₁-C₁₂ alkylamino (e.g., mono- or di-alkylamino)), and —C(O)—(C₄-C₆ N-heterocycloalkyl)

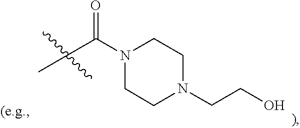 [SMILES: *C(=O)N1CCN(CCO)CC1]

wherein the C₄-C₆ N-heterocycloalkyl moiety of any of the preceding substituents is optionally substituted with C₁-C₃ alkyl or C₁-C₃ hydroxyalkyl.

Embodiment 46. The composition of Embodiment 45, wherein each terminating group is independently C₁-C₁₈ (e.g., C₄-C₁₈) alkylthiol, wherein the alkyl moiety is optionally substituted with one or more (e.g., one) substituents each independently selected from C₆-C₁₂ aryl (e.g., phenyl), C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

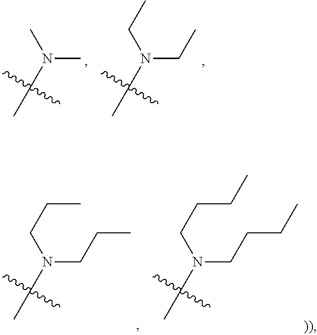

C₄-C₆ N-heterocycloalkyl (e.g., N-pyrrolidinyl

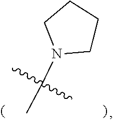 [SMILES: *N1CCCC1]

### N-piperidinyl

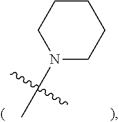 [SMILES: *N1CCCCC1]

### N-azepanyl

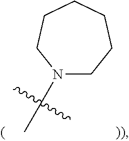 [SMILES: *N1CCCCCC1]

—OH, —C(O)OH, —C(O)N(C₁-C₃ alkyl)-(C₁-C₆ alkylene)-(C₁-C₁₂ alkylamino (e.g., mono- or di-alkylamino))

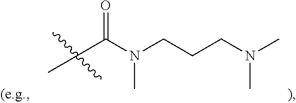 [SMILES: *C(=O)N(C)CCCN(C)C]

—C(O)N(C₁-C₃ alkyl)-(C₁-C₆ alkylene)-(C₄-C₆ N-heterocycloalkyl)

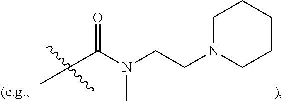 [SMILES: *C(=O)N(C)CCN1CCCCC1]

and —C(O)—(C₄-C₆ N-heterocycloalkyl)

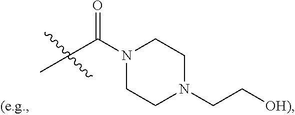

wherein the C₄-C₆ N-heterocycloalkyl moiety of any of the preceding substituents is optionally substituted with C₁-C₃ alkyl or C₁-C₃ hydroxyalkyl.

Embodiment 47. The composition of Embodiment 46, wherein each terminating group is independently C₁-C₁₈ (e.g., C₄-C₁₈) alkylthiol, wherein the alkyl moiety is optionally substituted with one substituent —OH.

Embodiment 48. The composition of Embodiment 46, wherein each terminating group is independently C₁-C₁₈ (e.g., C₄-C₁₈) alkylthiol, wherein the alkyl moiety is optionally substituted with one substituent selected from C₁-C₁₂ (e.g., C₁-C₈) alkylamino (e.g., C₁-C₆ mono-alkylamino (such as —NHCH₂CH₂CH₂CH₃) or C₁-C₈ di-alkylamino (such as

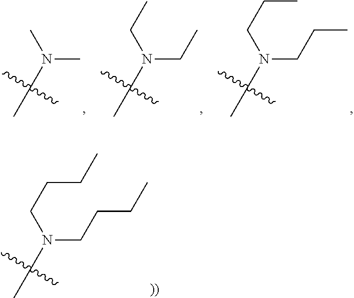

and C₄-C₆ N-heterocycloalkyl (e.g., N-pyrrolidinyl

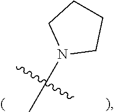 [SMILES: *N1CCCC1]

### N-piperidinyl

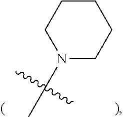 [SMILES: *N1CCCCC1]

### N-azepanyl

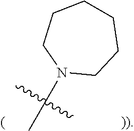 [SMILES: *N1CCCCCC1]

Embodiment 49. The composition of Embodiment 45, wherein each terminating group is independently C₁-C₁₈ (e.g., C₄-C₁₈) alkenylthiol or C₁-C₁₈ (e.g., C₄-C₁₈) alkylthiol.

Embodiment 50. The composition of Embodiment 47 or 49, wherein each terminating group is independently C₁-C₁₈ (e.g., C₄-C₁₈) alkylthiol.

Embodiment 51. The composition of Embodiment 50, wherein each terminating group is independently selected from the group consisting of:

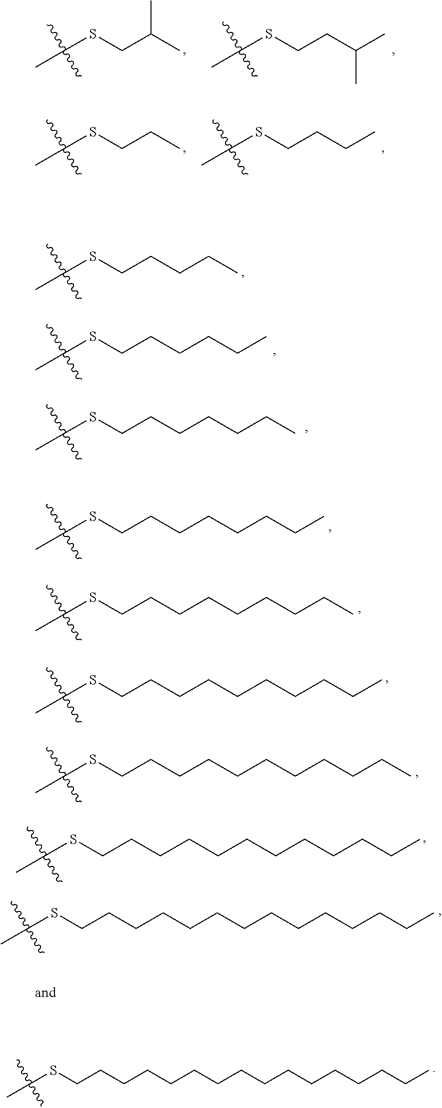

Embodiment 52. The composition of any one of Embodiments 5-44, wherein each terminating group is independently selected from those set forth in Table 3 or a subset thereof.

Embodiment 53. The composition of any one of Embodiments 1-4, wherein the ionizable cationic lipid is selected from those set forth in Table 4, or pharmaceutically acceptable salts thereof, or a subset of the lipids and the pharmaceutically acceptable salts thereof.

Embodiment 54. The composition of any one of Embodiments 1-4, wherein the ionizable cationic lipid is selected from those set forth in Table 4 or Table 5, or pharmaceutically acceptable salts thereof, or a subset of the lipids and the pharmaceutically acceptable salts thereof.

Embodiment 55. The composition of any one of Embodiments 1-54, wherein the lipid composition further comprises a phospholipid.

Embodiment 56. The composition of Embodiment 55, wherein the phospholipid is at a molar percentage from about 8% to about 23%.

Embodiment 57. The composition of any one of Embodiments 1-56, wherein the lipid composition further comprises a steroid or steroid derivative.

Embodiment 58. The composition of Embodiment 57, wherein the steroid or steroid derivative is at a molar percentage from about 15% to about 46%.

Embodiment 59. The composition of any one of Embodiments 1-58, wherein the ionizable cationic lipid is at a molar percentage from about 5% to about 30%.

Embodiment 60. The composition of any one of Embodiments 1-59, wherein the polymer-conjugated lipid is at a molar percentage from about 0.5% to about 10%.

Embodiment 61. The composition of any one of Embodiments 1-59, wherein the polymer-conjugated lipid is at a molar percentage from about 1% to about 10%.

Embodiment 62. The composition of any one of Embodiments 1-59, wherein the polymer-conjugated lipid is at a molar percentage from about 2% to about 10%.

Embodiment 63. The composition of any one of Embodiments 1-62, wherein the SORT lipid is at a molar percentage from about 20% to about 65%.

Embodiment 64. The composition of any one of Embodiments 1-63, wherein the therapeutic agent is a polynucleotide; and wherein a molar ratio of nitrogen in the lipid composition to phosphate in the polynucleotide (N/P ratio) is no more than about 20:1.

Embodiment 65. The composition of Embodiment 64, wherein the N/P ratio is from about 5:1 to about 20:1.

Embodiment 66. The composition of any one of Embodiments 1-65, wherein a molar ratio of the therapeutic agent to total lipids of said lipid composition is no more than about 1:1, 1:10, 1:50, or 1:100.

Embodiment 67. The composition of any one of Embodiments 1-66, wherein at least about 85% of said therapeutic agent is encapsulated in particles of said lipid compositions.

Embodiment 68. The composition of any one of Embodiments 1-67, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

Embodiment 69. The composition of any one of Embodiments 1-68, wherein said lipid composition has an apparent ionization constant (pKa) outside a range of 6 to 7.

Embodiment 70. The composition of Embodiment 69, wherein said apparent pKa of said lipid composition is of about 7 or higher.

Embodiment 71. The composition of Embodiment 69, wherein said apparent pKa of said lipid composition is of about 8 or higher.

Embodiment 72. The composition of Embodiment 69, wherein said apparent pKa of said lipid composition is from about 8 to about 13.

Embodiment 73. A method for targeted delivery of a therapeutic agent to a spleen cell, the method comprising (e.g., systemically) administering a composition according to any one of Embodiments 1-72, thereby providing an effective amount or activity of said therapeutic agent in said spleen cell of said subject that is at least 1.1-fold greater than a corresponding amount or activity of said therapeutic agent achieved in a lung cell of said subject.

Embodiment 74. The method of Embodiment 73, wherein the effective amount or activity of said therapeutic agent in said spleen cell of said subject that is at least 1.1-fold greater, at least 2.5-fold greater, at least 3.5-fold greater, at least 10-fold greater, at least 5.5-fold greater, at least 10-fold greater, at least 15-fold greater, at least 20-fold greater, or at least 50-fold greater than a corresponding amount or activity of said therapeutic agent achieved in a lung cell of said subject.

Embodiment 75. A method for targeted delivery of a therapeutic agent to a lung cell, the method comprising (e.g., systemically) administering a composition according to any one of Embodiments 1-72, thereby providing an effective amount or activity of said therapeutic agent in said lung cell of said subject that is at least 1.1-fold greater than a corresponding amount or activity of said therapeutic agent achieved in a spleen cell of said subject.

Embodiment 76. The method of Embodiment 75, wherein said effective amount or activity of said therapeutic agent in said lung cell of said subject is at least 1.1-fold greater, at least 5-fold greater, at least 10-fold greater, at least 15-fold greater, at least 18-fold greater, at least 20-fold greater, or at least about 50-fold greater than a corresponding amount or activity of said therapeutic agent achieved in a liver cell of said subject.

### EXAMPLES

### Example 1: Preparation of DOTAP or DODAP Modified Lipid Nanoparticles

Lipid nanoparticles (LNPs) are the most efficacious carrier class for in vivo nucleic acid delivery. Historically, effective LNPs are composed of 4 components: an ionizable cationic lipid, zwitterionic phospholipid, cholesterol, and lipid poly(ethylene glycol) (PEG). However, these LNPs result in only general delivery of nucleic acids, rather than organ or tissue targeted delivery. LNPs typically delivery RNAs only to the liver. Therefore, new formulations of LNPs were sought in an effort to provide targeted nucleic acid delivery.

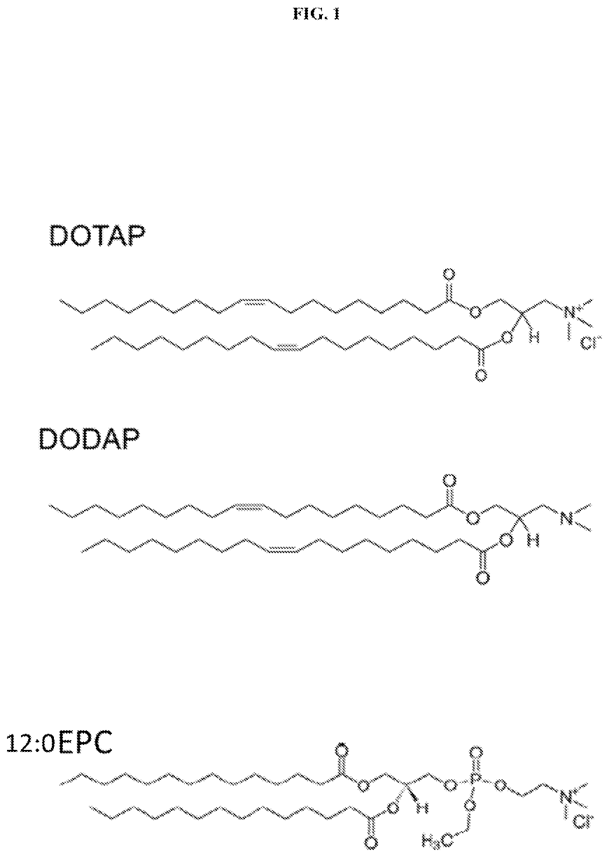

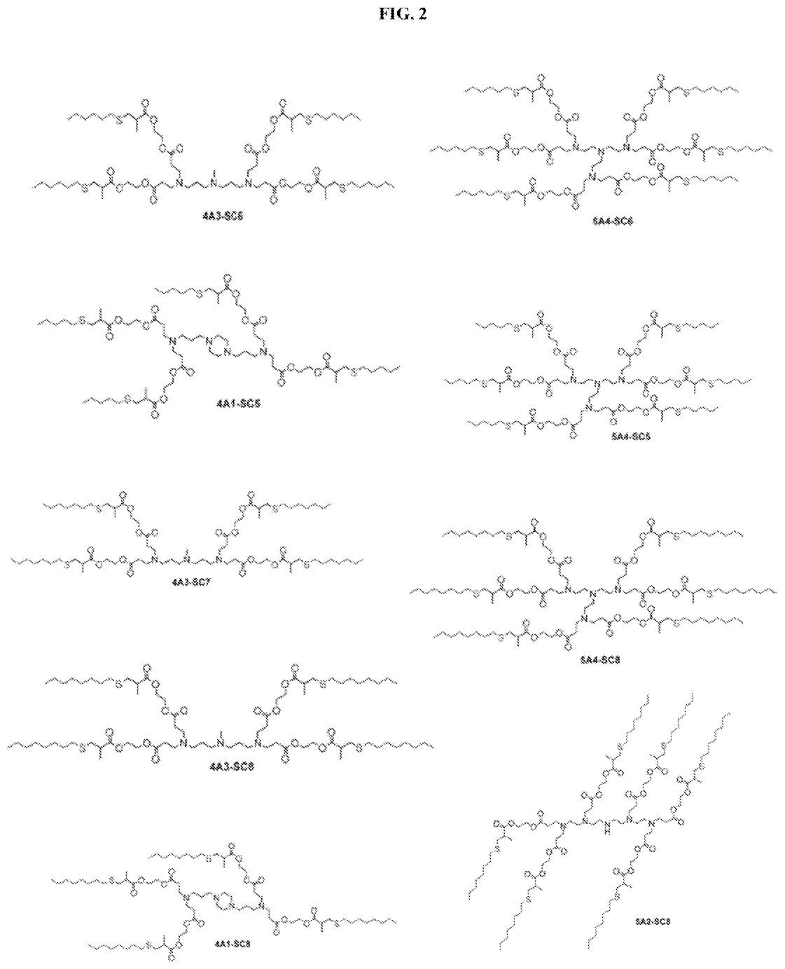

The four canonical types of lipids were mixed in a 15:15:30:3 molar ratio, with or without the addition of a permanently cationic lipid. Briefly, LNPs were prepared by mixing a dendrimer or dendron lipid (ionizable cationic), DOPE (zwitterionic), cholesterol, DMG-PEG, and DOTAP (permanently cationic). Alternatively, DOTAP can be substituted for DODAP to generate an LNP comprising DODAP. The structure of DODAP and DODAP are shown in FIG. 1. Various dendrimer or dendron lipids that may be used are shown in FIG. 2.

For preparation of the LNP formulation, a dendrimer or dendron lipid, DOPE, Cholesterol and DMG-PEG were dissolved in ethanol at desired molar ratios. The mRNA was dissolved in citrate buffer (10 mM, pH 4.0). The mRNA was then diluted into the lipids solution to achieve a weight ratio of 40:1 (total lipids: mRNA) by rapidly mixing the mRNA into the lipid solution at a volume ratio of 3:1 (mRNA: lipids, v/v). This solution was then incubated for 10 min at room temperature. For formation of DOTAP modified LNP formulations, mRNA was dissolved in 1×PBS or citrate buffer (10 mM, pH 4.0), and mixed rapidly into ethanol containing 5A2-SC8, DOPE, Cholesterol, DMG-PEG and DOTAP, fixing the weight ratio of 40:1 (total lipids:mRNA) and volume ratio of 3:1 (mRNA:lipids). Formulations are named X % DOTAP Y (or X % DODAP Y) where X represents the DOTAP (or DODAP) molar percentage in total lipids, and Y represents the type of dendrimer or dendron lipid. Alternatively, formulation may be named Y X % DOTAP or Y X % DODAP where X represents the DOTAP (or DODAP) molar percentage in total lipids, and Y represents the type of dendrimer or dendron lipid.

### Example 2: SORT LNP Stability

Example lipid compositions as described herein were generated using either a microfluidic mixing method or a cross/tee mixing method and tested for stability. The physical characteristics including size, polydispersity index (PDI) and zeta-potential were characterized by dynamic light scattering (DLS) from various example lipid (LNP) compositions (3 separate measurements for each formulation) and illustrated in Table 7.

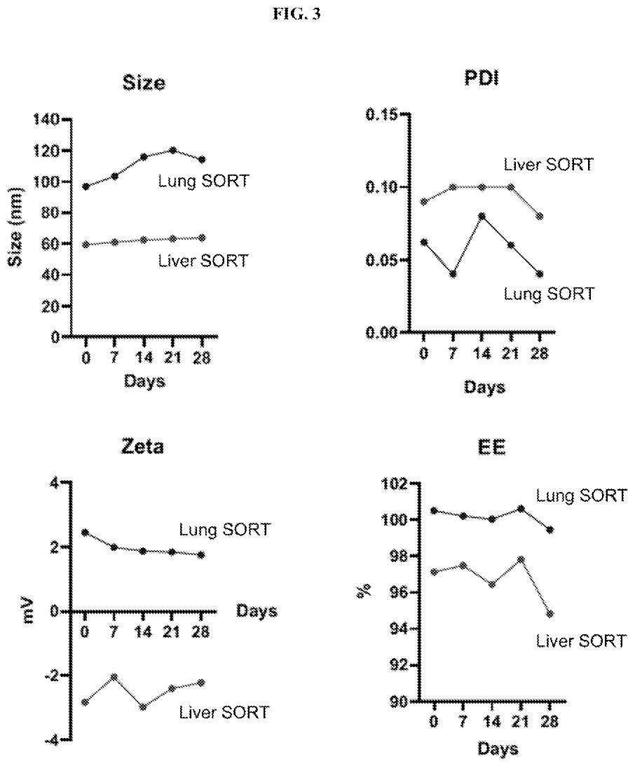

The encapsulation efficiency was tested using a Ribogreen RNA assay (Zhao et al., 2016). Briefly, mRNA was encapsulated with >95% efficiency in LNPs when the mRNA was dissolved in acidic buffer (10 mM citrate, pH 4). The characteristics were observed over 28 days for the two types of LNPs (5A2-SC8 with 20% DODAP (“Liver-SORT) and 5A2-SC8 with 50% DOTAP (“Lung-SORT”)). FIG. 3 illustrates changes in the physical characteristics of the lipid compositions over a duration of 28 days.

TABLE 7
SORT LNP characteristics

| | Size | | Zeta | Encapsulation |
|---|---|---|---|---|
| | (nm) | PDI | (mV) | Efficiency (%) |
| Lung-SORT - | 82.3 | 0.10 | 3.0 | 100 |
| microfluidic | | | | |
| Lung-SORT - | 78.1 | 0.09 | 2.2 | 100 |
| cross/tee mixing | | | | |
| Liver-SORT - | 59.1 | 0.10 | −2.3 | 97 |
| microfluidic | | | | |
| Liver-SORT - | 60.0 | 0.11 | −30 | 96 |
| cross/tee mixing | | | | |

### Example 3: Lung-SORT IV Study

This example compiles several studies of administration of Lung-SORTs intravenously and systemically to multiple species (e.g., mice, rats, dogs, and non-human primate (NHP) (e.g., Rhesus macaques, Cynomolgus macaques)).

### Summary

Most of the test subjects were administered a single dose of luciferase mRNA encapsulated in a Lung-SORT LNP (e.g., 5A2-SC8 with 50% DOTAP). Some of the test subjected were administered two doses. The low doses administered to dogs and NHP was translated from mice studies. In this study, Whole animal imaging, organ imaging, blood chemistry, hematology, immunoreactivity, complement, and tolerability parameters were assessed. The Lung-SORT LNP showed selectivity across species to target lung and avoid other organ and tissues. Various DOTAP alternative LNPs (e.g., 14:0 TAP) were tested in vivo (e.g., mice) and in vitro, e.g., in hBEs and exhibit improved tolerability, higher potency, and similarly or better selectivity. Animal experiments (e.g., in dogs) also tested used a second Lung-SORT with a 14:0 TAP (SORT lipid) (“RTX0031”), formulation highly potent, very selective for lung targeting (results similar for IV bolus and IV infusion, with no signs of in-infusion related reactions (IRRs).

The example “Lung-SORT LNP” tested herein was a 5-component lipid nanoparticle composition comprising about 11.9% 5A2-SC8 (ionizable cationic lipid), about 50% DOTAP (SORT lipid), about 11.9% DOPE lipid, about 23.8% cholesterol, and about 2.4% DMG-PEG (PEG conjugated lipid), wherein each lipid component is defined as mol % of the total lipid composition.

The example DOTAP alternative LNP replaces the DOTAP (SORT lipid) with 14:0 TAP as the SORT lipid (referred to “RTX0031 LNP”). This RTX0031 LNP composition tested herein was a 5-component lipid nanoparticle composition comprising about 14.3% 5A2-SC8 (ionizable cationic lipid), about 40% 14:0 TAP (SORT lipid), about 14.3% DOPE, about 28.6% cholesterol, and about 2.8% DMG-PEG (PEG conjugated lipid), wherein each lipid component is defined as mol % of the total lipid composition.

### NHP IV Lung-SORT LNP Study

Lung-SORT LNP containing 0.1 mg/kg luciferase mRNA were delivered to NHPs via IV bolus over 5 min without any pre-medication (e.g., steroids). Whole body bioluminescence imaging was taken 4 hr post IV bolus administration. The study showed the Lung-SORT LNP formulation was active in NHPs and highly potent as the delivery of mRNA was outside the liver and significant signal was observed in the lungs. Additionally, tolerability of the Lung-SORT LNP was assessed in NHPs. Without wishing to be bound to any theory, DOTAP and/or the method of administration may play a factor in the tolerability.

The RTX0031 LNP was tested tested herein. Additionally, therapeutically relevant parameters were further tested (e.g., slow infusion with premedication as needed).

TABLE 8
Parameters of Beagle Lung SORT LNP
(5A2-SC8 with 50% DOTAP) Study

| | |
|---|---|
| Status | In-life complete |
| Formulation | Lung-SORT LNP |
| | (5A2-SC8 with 50% DOTAP) |
| Method of Administration | IV bolus (0.5 mL, 5 min push) |
| Dose | 0.01 mg/kg |
| N | 2 (1 M, 2 yrs old, 8-10 kg) |
| Readouts | Blood chem, hematology, cytokines, |
| | complement, IVIS of explanted organs. |

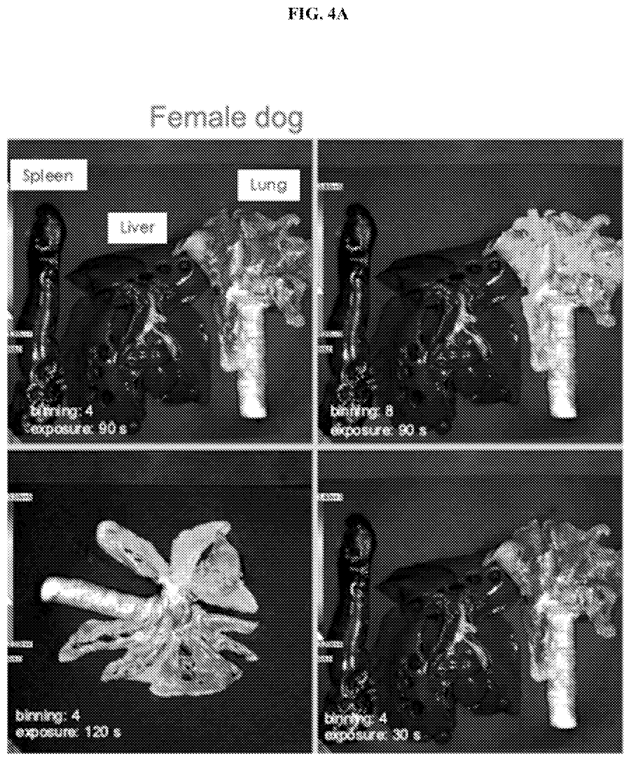

FIGS. 4A and 4B shows IVIS organ imaging of spleen, liver and lung of a female dog and male dog after the administration of Lung-SORT LNP (5A2-SC8 with 50% DOTAP). The higher signal seen in the lungs showed that the Lung-SORT LNP was selective for lung delivery.

Both dogs showed signs consistent with mild infusion-related reactions. The female (first) dog had very mild tachycardia compared to her baseline. The blood chemistry of the female dog showed only small changes, 2-fold increase in AST, CK, and LDH. The hematology assessment showed no significant changes. The male dog showed mucosal hyperemia and mild lethargy/muscle weakness (ventroflexion of the neck) in the first 15 min post-injection, which resolved over the first half hour post-infusion and were not severe enough to warrant intervention. The male (second) dog had mild bradycardia compared to the baseline and was observed in the first hour-post infusion.

TABLE 9
Cynomolgus Lung-SORT LNP (5A2-
SC8 with 50% DOTAP) Study

| | |
|---|---|
| Status | In-life complete |
| Formulation | Lung-SORT LNP |
| | 5A2-SC8 with 50% DOTAP |
| Method of Administration | IV bolus (5 min push) |
| Dose | 0.01 mg/kg |
| Number of Doses | 2 (3 weeks apart) |
| N | 2 (Female, ~3 kg) |
| Readouts | Blood chem, hematology, cytokines, |
| | complement, IVIS of explanted organs. |

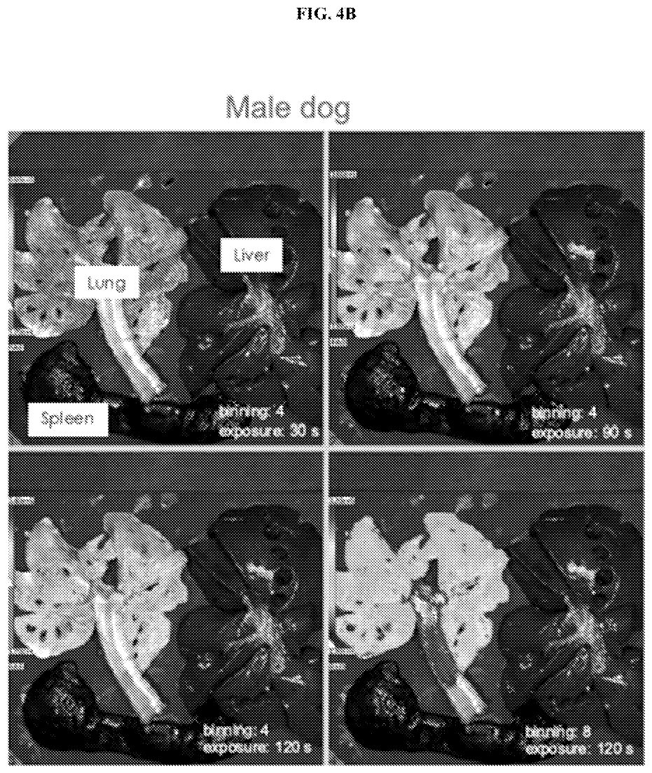

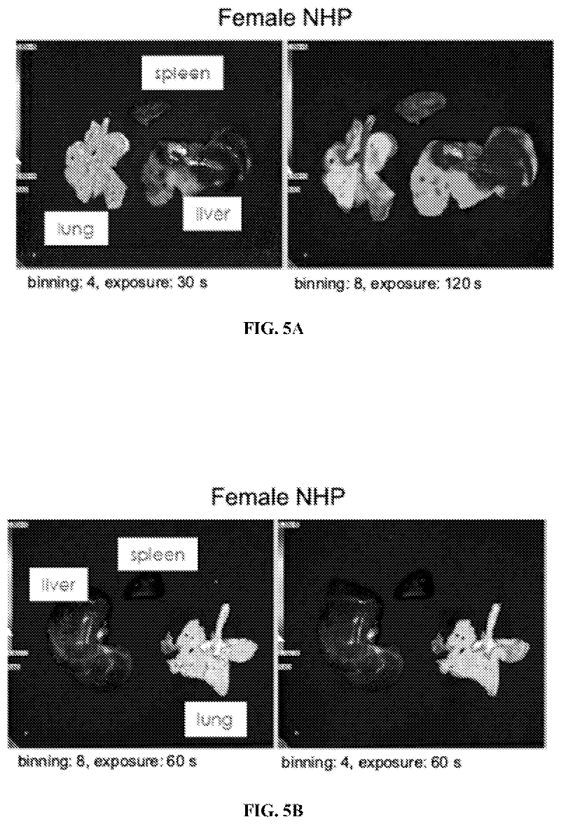

FIGS. 5A and 5B shows IVIS organ imaging of spleen, liver and lung of both Cynomolgus NHPs after the administration of the Lung-SORT LNP (5A2-SC8 with 50% DOTAP). The higher signal seen in the lungs showed that the Lung-SORT LNP was selective for lung delivery.

No adverse reactions were observed during or after the first or second administration of the Lung-SORT formulation. The blood chemistry of the Cynomolgus NHPs test subjects showed an increase in AST, ALT, CK and LDH after the first dose and showed an increase in LDH and CO₂ after the second dose. The hematology of the Cynomolgus NHPs test subjects showed an increase in neutrophils and decrease in lymphocytes and eosinophils after the first and second doses.

Lung-SORT LNP (5A2-SC8 with 50% DOTAP) Delivered as IV Bolus in Rats, Dogs, Rhesus Macaques and Cynomolgus Macaques

The Lung-SORT LNP was well tolerated when delivered as IV bolus without any premeds and shown no adverse reactions during 2 administrations to Cynomolgus macaques and only mild signs of IRRs in dogs after a single administration. The AST, CK, LDH, and changes in neutrophils/lymphocytes were good indication of tolerability blood markers across the tested species.

The tested rats did not provide as favorable results with differences in potency and toxicity in comparison to the tested mice, dogs, and NHPs subjects, only showing signs of toxicity at 1 mg/mL doses.

### RTX0031 LNP (DOTAP Alternative for Lung-SORT) in Mice Study

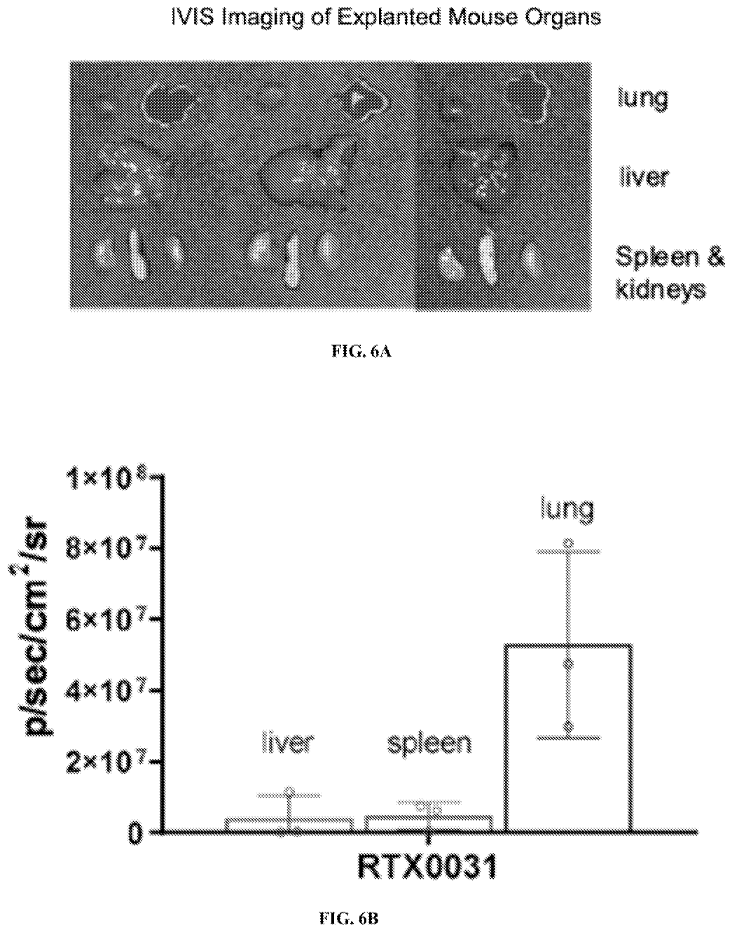

FIG. 6A shows IVIS organ imaging of spleen, liver, kidneys and lung of three mice after the administration of luciferase mRNA formulated with the RTX0031 LNP after 5 hrs. The higher signal seen in the lungs compared to the liver, spleen and kidneys show that the RTX0031 LNP was selective for lung delivery. FIG. 6B quantitatively displays the signal obtained at the lungs, spleen, and liver of the 3 mice. The in vivo study may provide information that RTX0031 LNP had improved tolerability and potency.

RTX0031 LNP (14:0 TAP as SORT Lipid) Versus Lung-SORT LNP (DOTAP as SORT Lipid) in hBEs Study

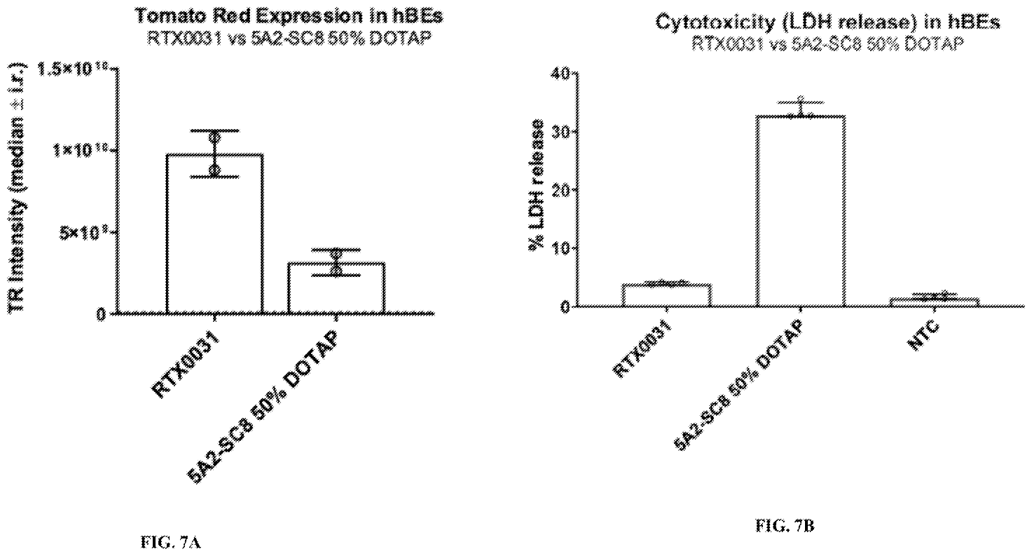

RTX0031 LNP and Lung-SORT LNP were tested in hBEs for tolerability and potency. The hBEs were dosed with apical liquid with 12 ug of formulated Tomato red (TR) mRNA formulated in the Lung-SORT and RTX0031 LNP formulations. The TR expression was assessed by fluorescence microscopy 24 hrs post treatment. FIG. 7A shows the TR intensity expressed in the treated hBEs of the two LNPs compositions tested. The RTX0031 LNP showed higher TR intensity compared to the Lung-SORT LNP (5A2-SC8 with 50% DOTAP) formulation. Additionally, the cytotoxicity (LDH release) was assessed after 48 hrs post treatment. FIG. 7B shows the % LDH released from the treated hBEs of the two LNP compositions. The Lung-SORT LNP (5A2-SC8 with 50% DOTAP) formulation showed a higher % of LDH released than the RTX0031 LNP formulation. The in vitro study may provide information that RTX0031 LNP formulation had improved tolerability and potency.

TABLE 10
LNP Study in Beagles with

| | |
|---|---|
| Status | In-life complete |
| Formulation | RTX0031 LNP |
| | (40% 14:0 TAP) |
| Method of | 1) IV bolus, 5 min push |
| Administration^{1, 2} | 2) IV infusion, ~30 min + premed |
| Dose | 0.01 mg/kg* |
| Number of Doses | 2 (3 weeks apart) |
| N | 4 (2 per group) |
| Readouts | Blood chem, hematology, cytokines, |
| | complement, IVIS of explanted organs. |

¹Injection volume: 0.5 mL for IV bolus, 10-15 mL for IV infusion
²Pre-medications to mitigate IRRs were: dexamethasone 0.1 mg/kg IV, acetaminophen 10 mg/kg PO, Diphenhydramine 1 mg/kg IV, Famotidine 0.5 mg/kg IV slow bolus
*RTX0031 LNP was about 2-fold more potent than the Lung-SORT LNP (5A2-SC8 with 50% DOTAP) in mice

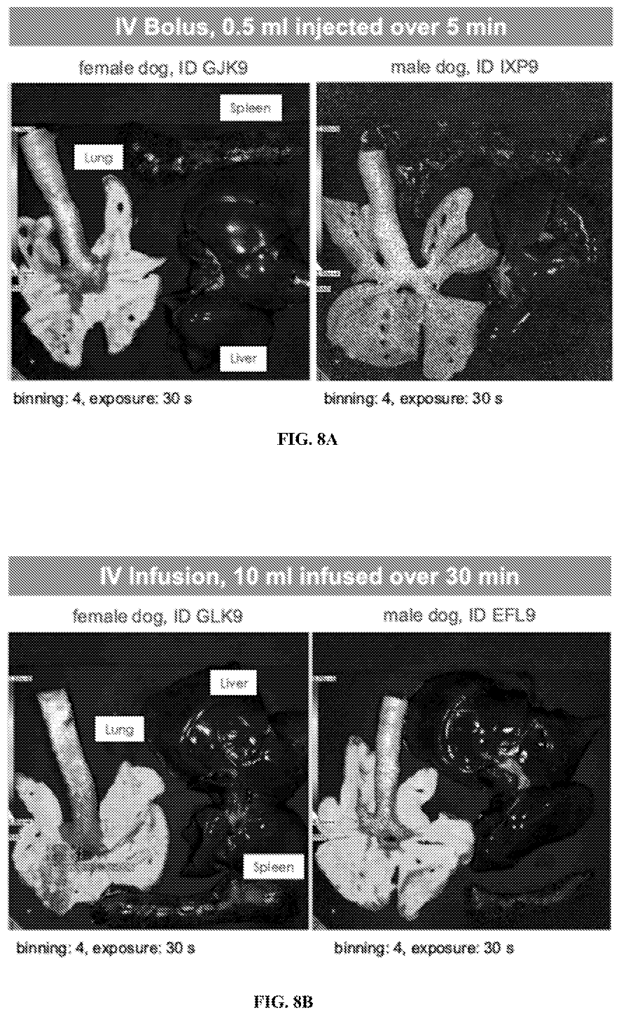

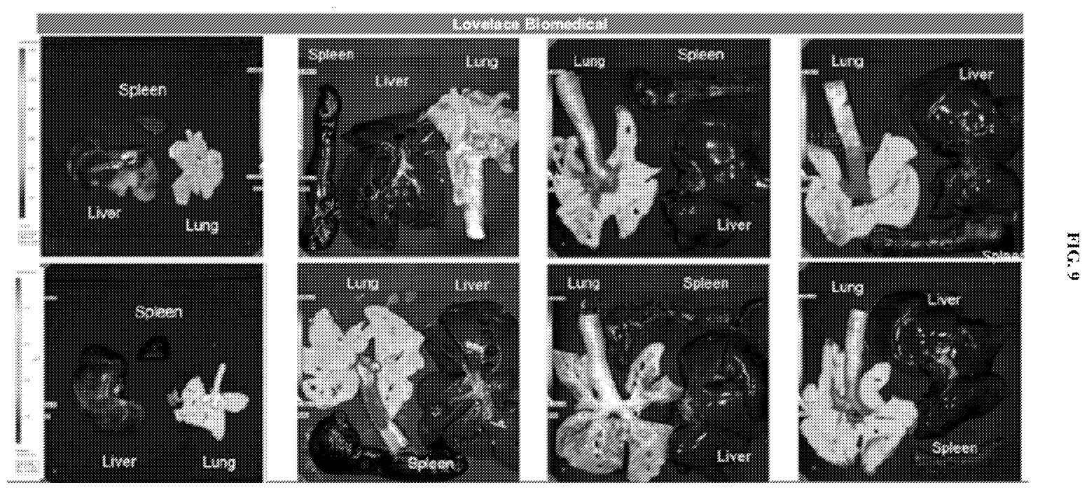

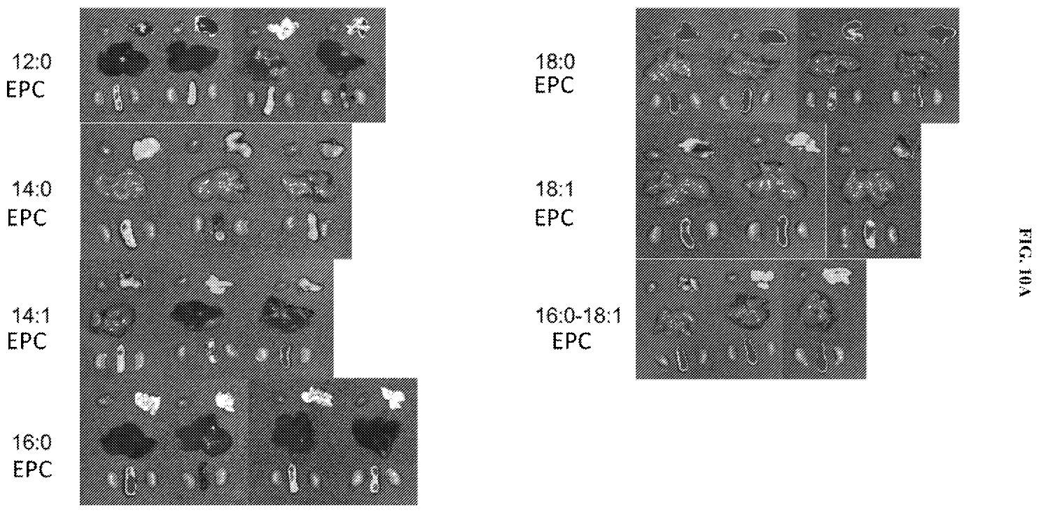

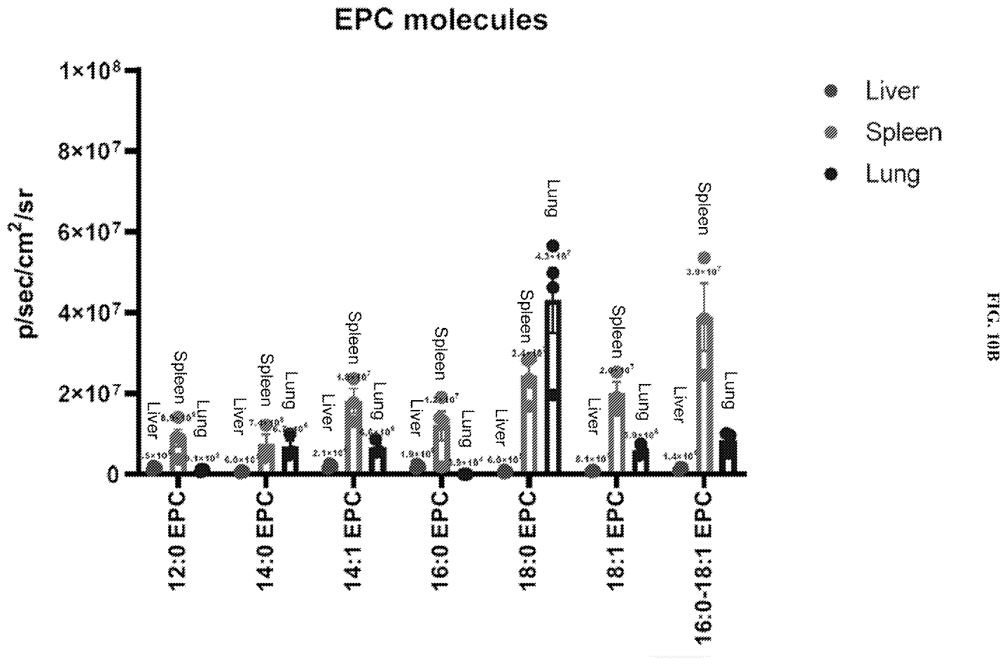

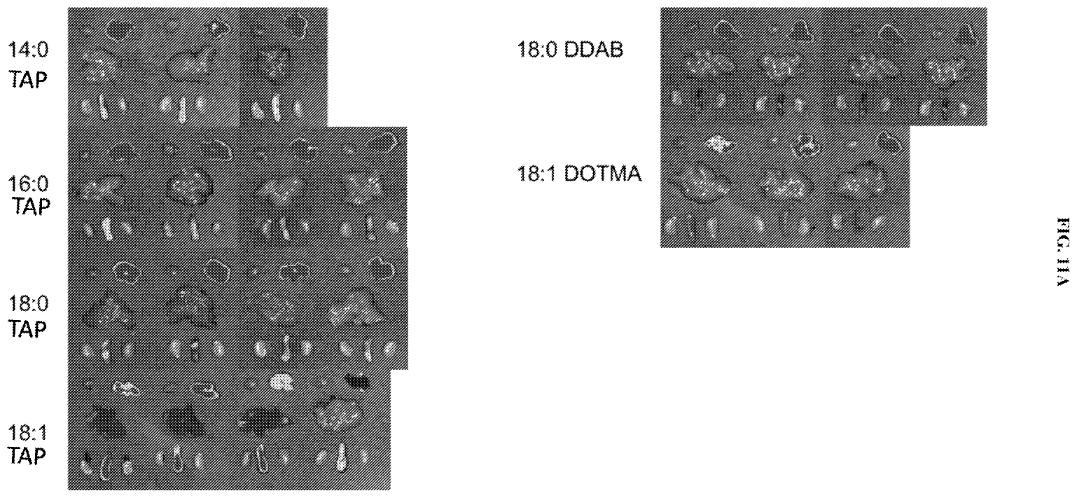

FIG. 8A shows IVIS organ imaging of spleen, liver, and lung of two beagles after the IV bolus administration of luciferase mRNA formulated in the RTX0031 LNP. The male dog had some hypersalivation shortly after the TA treatment that was observed for less than one to two minutes. Female dog had no signs of adverse reactions during or after TA treatment. For both dogs, temperatures, heart rate, and respiratory rates showed only minor changes and mainly related to stress due to handling. The female dog experienced a seizure during luciferin administration. The blood chemistry showed an increase in ALT by ˜3-old in the male dog. The hematology showed only small changes, particularly decreases in lymphocytes and eosinophils in the male dog.

FIG. 8B shows IVIS organ imaging of spleen, liver, and lung of two beagles after the IV infusion of luciferase mRNA formulated in the RTX0031 LNP with pre-meds. Both dogs did not show any clinical observations or signs of adverse reactions during or after administration of TA. One of the dogs experienced a seizure during luciferin administration. The blood chemistry showed an increase in ALT (2-fold), CK (3-fold) and LDH (5-fold) in the female dog. The hematology only showed small changes, particularly decreases in lymphocytes and eosinophils.

FIG. 9 shows a compiled panel of IVIS organ imaging of spleen, liver, and lung of dogs and NHPs as seen in FIGS. 4A, 4B, 5A, 5B, 8A, and 8B.

TABLE 11
Compiled Beagle and NHP Lung-SORT LNP and RTX0031 LNP Study Models

| | Model | | | | |
|---|---|---|---|---|---|
| | Rhesus | Cynomolgus | Beagle | Beagle | Beagle |
| | (M) | (F) | (1M, 1F) | (1M, 1F) | (1M, 1F) |
| Formulation | Lung SORT LNP | Lung SORT LNP | Lung SORT LNP | RTX0031 LNP | RTX0031 |
| | (5A2-SC8 50%) | (5A2-SC8 50%) | (5A2-SC8 50%) | (14:0 TAP) | (14:0 TAP) |
| N | 1 | 2 | 2 | 2 | 2 |
| Delivery | IV bolus | IV bolus | IV bolus | IV bolus | IV infusion |
| | (5 min) | (5 min) | (5 min) | (5 min) | (30 min) + |
| | single dose | Dosed 2x, 3 | Single dose | Single dose | premeds, |
| | | wks apart | | | single dose |
| IRRs | Yes, moderate | No | Yes (in 1 of 2), | Yes (in 1 of 2), | No |
| | to severe | | mild | very mild | |

### Example 4: Organ Selective Tropism of Spleen SORT Formulations and Lung SORT Formulations

Luc mRNA/LNP comprising 5A2-SC8 lipid and 40% of a SORT lipid as described in Table 12 were intravenously administered to mice at 0.05 mpk dose. Each SORT lipid investigated in Table 12 had n=4 mice per group. After 5 hr post administration of LNPs, the organs were excised and the kidneys, lungs, spleen, and liver were IVIS imaged to determine the selective organ tropism of the differ SORT lipids.

The exemplary LNP tested herein were 5 component lipid nanoparticle composition comprising about 14.3% 5A2-SC8 (ionizable cationic lipid), about 40% SORT lipid from Table 12, about 14.3% DOPE, about 28.6% cholesterol, and about 2.8% DMG-PEG (PEG conjugated lipid), wherein each lipid component is defined as mol % of the total lipid composition

TABLE 12
SORT LNPs with different SORT lipid

| SORT Lipid | Size (nm) | PDI | Encapsulation (%) |
|---|---|---|---|
| 12:0 EPC | 45.3 | 0.16 | 97 |
| 14:0 EPC | 50.7 | 0.10 | 96 |
| 14:1 EPC | 52.9 | 0.13 | 94 |
| 16:0 EPC | 60.9 | 0.16 | 93 |
| 18:0 EPC | 80.3 | 0.08 | 95 |
| 18:1 EPC | 56.8 | 0.10 | 95 |
| 16:0-18:1 EPC | 50.7 | 0.09 | 95 |
| 14:0 TAP | 104.4 | 0.15 | 96 |
| 16:0 TAP | 133.9 | 0.12 | 97 |
| 18:0 TAP | 241.0 | 0.13 | 96 |
| 18:1 DOTAP | 61.0 | 0.12 | 95 |
| 18:0 DDAB | 133.8 | 0.17 | 96 |
| 18:1 DOTMA | 66.8 | 0.03 | 95 |

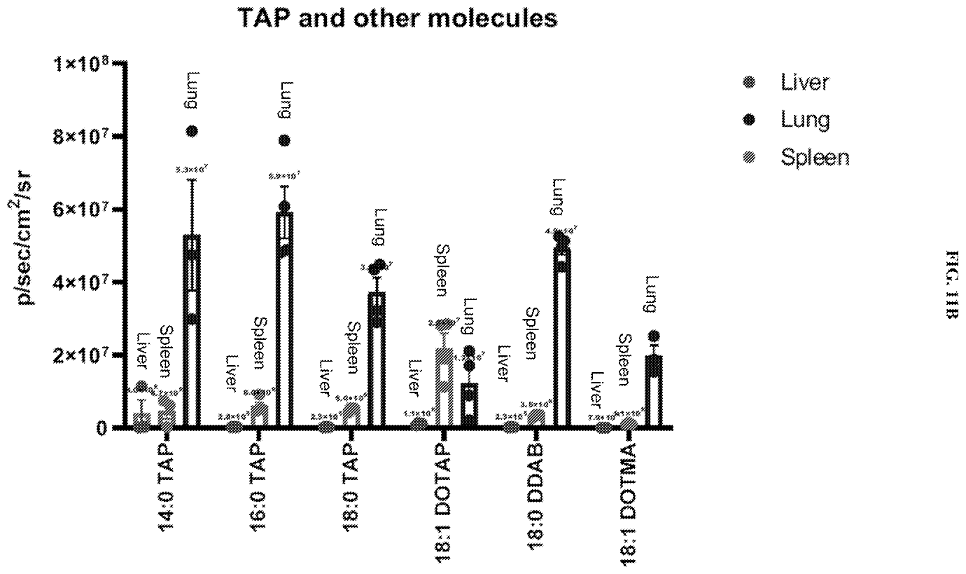

The IVIS images of FIG. 11A displays the varying organ selectivity of 12:0 EPC, 14:0 EPC, 14:1 EPC, 16:0 EPC, 18:0 EPC, 18:1 EPC, and 16:0-18:1 EPC SORT lipids of SORT LNPs delivered to the mice. The higher intensities localized in the spleen and lungs vs. the liver displays organ targeting tropism of SORT LNPs and influence of SORT lipids. FIG. 11B quantitively displays the IVIS data for the liver, spleen, and lungs of the mice with the different SORT lipids tested. Each tested EPC lipid has a higher selectivity to spleen and lungs vs. liver in all cases tested as seen with the higher luminescence intensity. Certain EPC lipids provides more effective delivery of the payload and levels of selectivity between the lungs and spleen.

The IVIS images of FIG. 12A displays the varying organ selectivity of 14:0 TAP, 16:0 TAP, 18:0 TAP, 18:1, TAP, 18:0 DDAB, and 18:1 DOTMA SORT lipids of SORT LNPs delivered to the mice. The higher intensities localized in the lungs vs. the liver and spleen displays organ targeting tropism of SORT LNPs and influence of SORT lipids. FIG. 12B quantitively displays the IVIS data for the liver, spleen, and lungs of the mice with the different SORT lipids tested. Most of the tested SORT lipid has a higher selectivity to lungs vs. liver and spleen as seen with the higher luminescence intensity. Certain SORT lipids provide more effective delivery of the payload and levels of selectivity between the lungs and spleen.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A composition formulated for systemic (e.g., intravenous) administration, the composition comprising a therapeutic agent assembled with a lipid composition that comprises:
(i) an ionizable cationic lipid;
(ii) a polymer-conjugated lipid; and
(iii) a selective organ targeting (SORT) lipid has the structure of Formula (IA), or a pharmaceutically acceptable salt, stereoisomer, tautomer thereof: 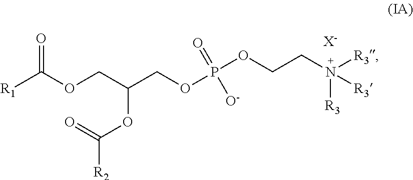
wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

2. The composition of claim 1, wherein the SORT lipid having the structure of Formula (IA) is selected from the group consisting of: 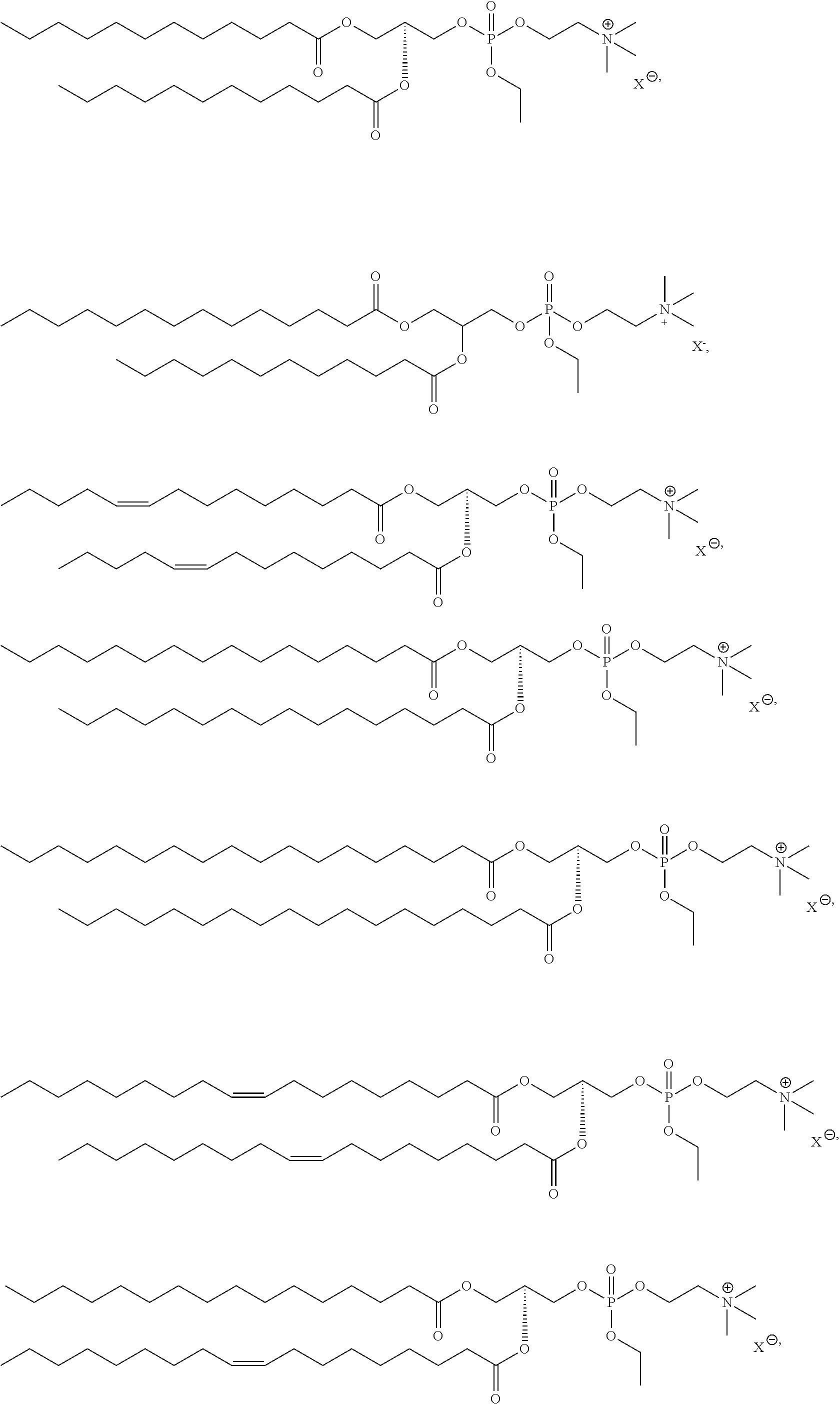
and a combination thereof.

3. A composition formulated for systemic (e.g., intravenous) administration, the composition comprising a therapeutic agent assembled with a lipid composition that comprises:
(i) an ionizable cationic lipid;
(ii) a polymer-conjugated lipid; and
(iii) a selective organ targeting (SORT) lipid has the structure of Formula (S-III), or a pharmaceutically acceptable salt, stereoisomer, tautomer thereof: 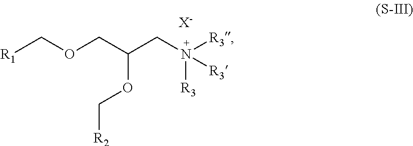
wherein:
R₁ and R₂ are each independently alkyl_{(C8-C24)}, alkenyl_{(C8-C24)}, or a substituted version of either group;
R₃, R₃′, and R₃″ are each independently alkyl_{(C≤6)} or substituted alkyl_{(C≤6)}; and
X⁻ is a monovalent anion.

4. The composition of claim 3, wherein the SORT lipid having the structure of Formula (S-III) is

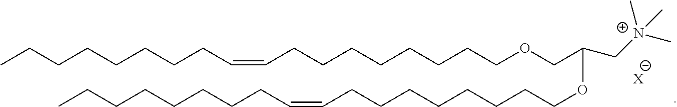

5. The composition of claim 1 or 2, wherein the ionizable cationic lipid is a dendrimer or dendron of a generation (g) having a structural formula: 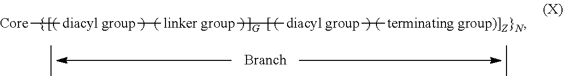
or a pharmaceutically acceptable salt thereof, wherein:
(a) the core comprises a structural formula (X_{Core}): 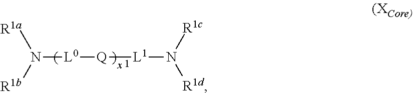
wherein:
Q is independently at each occurrence a covalent bond, —O—, —S—, —NR²—, or —CR^{3a}R^{3b}—;
R² is independently at each occurrence R^{1g} or -L²-NR^{1e}R^{1f};
R^{3a} and R^{3b} are each independently at each occurrence hydrogen or an optionally substituted (e.g., C₁-C₆, such as C₁-C₃) alkyl;
R^{1a}, R^{1b}, R^{1c}, R^{1d}, R^{1e}, R^{1f}, and R^{1g} (if present) are each independently at each occurrence a point of connection to a branch, hydrogen, or an optionally substituted (e.g., C₁-C₁₂) alkyl;
L⁰, L¹, and L² are each independently at each occurrence selected from a covalent bond, (e.g., C₁-C₁₂, such as C₁-C₆ or C₁-C₃) alkylene, (e.g., C₁-C₁₂, such as C₁-C₈ or C₁-C₆) heteroalkylene (e.g., C₂-C₈ alkyleneoxide, such as oligo(ethyleneoxide)), [(e.g., C₁-C₆) alkylene]-[(e.g., C₄-C₆) heterocycloalkyl]-[(e.g., C₁-C₆) alkylene], [(e.g., C₁-C₆) alkylene]-(arylene)-[(e.g., C₁-C₆) alkylene] (e.g., [(e.g., C₁-C₆) alkylene]-phenylene-[(e.g., C₁-C₆) alkylene]), (e.g., C₄-C₆) heterocycloalkyl, and arylene (e.g., phenylene); or,
alternatively, part of L¹ form a (e.g., C₄-C₆) heterocycloalkyl (e.g., containing one or two nitrogen atoms and, optionally, an additional heteroatom selected from oxygen and sulfur) with one of R^{1c} and R^{1d}; and
x¹ is 0, 1, 2, 3, 4, 5, or 6; and
(b) each branch of the plurality (N) of branches independently comprises a structural formula (X_{Branch}): 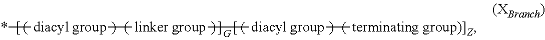
wherein:
* indicates a point of attachment of the branch to the core;
g is 1, 2, 3, or 4;
Z=2^{(g-1)};
G=0, when g=1; or G=Σᵢ₌₀^{i=g-2}, when g≠1;
(c) each diacyl group independently comprises a structural formula 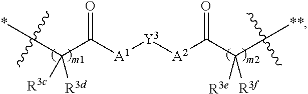
wherein:
* indicates a point of attachment of the diacyl group at the proximal end thereof,
** indicates a point of attachment of the diacyl group at the distal end thereof,
Y³ is independently at each occurrence an optionally substituted (e.g., C₁-C₁₂);
alkylene, an optionally substituted (e.g., C₁-C₁₂) alkenylene, or an optionally substituted (e.g., C₁-C₁₂) arenylene;
A¹ and A² are each independently at each occurrence —O—, —S—, or —NR⁴—, wherein:
R⁴ is hydrogen or optionally substituted (e.g., C₁-C₆) alkyl;
m¹ and m² are each independently at each occurrence 1, 2, or 3; and
R^{3c}, R^{3d}, R^{3e}, and R^{3f} are each independently at each occurrence hydrogen or an optionally substituted (e.g., C₁-C₈) alkyl; and
(d) each linker group independently comprises a structural formula 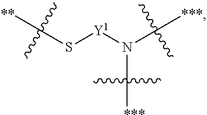
wherein:
** indicates a point of attachment of the linker to a proximal diacyl group;
*** indicates a point of attachment of the linker to a distal diacyl group; and
Y₁ is independently at each occurrence an optionally substituted (e.g., C₁-C₁₂) alkylene, an optionally substituted (e.g., C₁-C₁₂) alkenylene, or an optionally substituted (e.g., C₁-C₁₂) arenylene; and
(e) each terminating group is independently selected from optionally substituted (e.g., C₁-C₁₈, such as C₄-C₁₈) alkylthiol, and optionally substituted (e.g., C₁-C₁₈, such as C₄-C₁₈) alkenylthiol.

6. The composition of claim 5, wherein x¹ is 0, 1, 2, or 3.

7. The composition of claim 5, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one or more substituents each independently selected from —OH, C₄-C₈ (e.g., C₄-C₆) heterocycloalkyl (e.g., piperidinyl 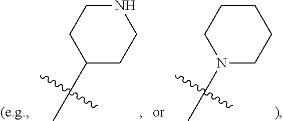 [SMILES: *C1CCNCC1 ; *N1CCCCC1]
N—(C₁-C₃ alkyl)-piperidinyl 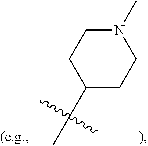 [SMILES: *C1CCN(C)CC1]
piperazinyl 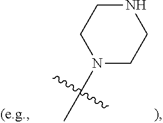 [SMILES: *N1CCNCC1]
N—(C₁-C₃ alkyl)-piperadizinyl 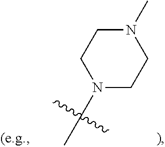 [SMILES: *N1CCN(C)CC1]
morpholinyl 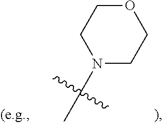 [SMILES: *N1CCOCC1]
N-pyrrolidinyl 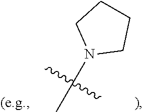 [SMILES: *N1CCCC1]
pyrrolidinyl 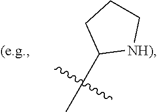
or N—(C₁-C₃ alkyl)-pyrrolidinyl 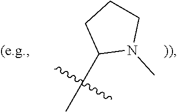
(e.g., C₆-C₁₀) aryl, and C₃-C₅ heteroaryl (e.g., imidazolyl 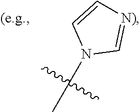
or pyridinyl

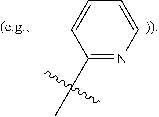 [SMILES: *c1ccccn1]

8. The method of claim 7, wherein R^{1a}, R^{1b}, R^{1c}, R^{1d}, R^{1e}, R^{1f}, and R^{1g} (if present) are each independently at each occurrence a point of connection to a branch (e.g., as indicated by *), hydrogen, or C₁-C₁₂ alkyl (e.g., C₁-C₈ alkyl, such as C₁-C₆ alkyl or C₁-C₃ alkyl), wherein the alkyl moiety is optionally substituted with one substituent —OH.

9. The composition of claim 5, wherein R^{3a} and R^{3b} are each independently at each occurrence hydrogen.

10. The composition of claim 5, wherein the plurality (N) of branches comprises at least 3 (e.g., at least 4, or at least 5) branches.

11. The composition of claim 5, wherein g=1; G=0; and Z=1.

12. The composition of claim 11, wherein each branch of the plurality of branches comprises a structural formula

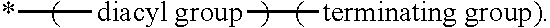

13. The composition of claim 5, wherein g=2; G=1; and Z=2.

14. The composition of claim 13, wherein each branch of the plurality of branches comprises a structural formula

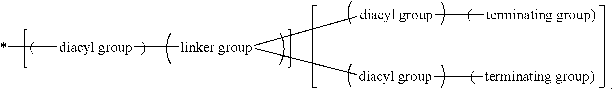

15. The composition of claim 5, wherein the core is selected from those set forth in Table 1 or a subset thereof.

16. The composition of claim 5, wherein the core comprises a structural formula selected from the group consisting of: 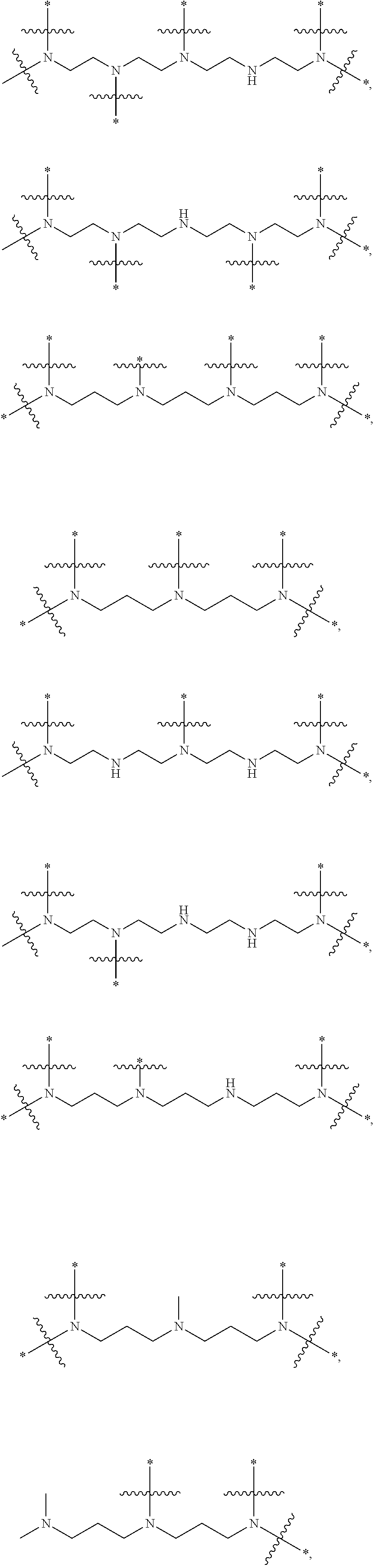
and pharmaceutically acceptable salts thereof, wherein * indicates a point of attachment of the core to a branch of the plurality of branches.

17. The composition of claim 5, wherein the core has the structure 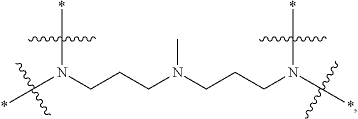
wherein * indicates a point of attachment of the core to a branch of the plurality of branches or H.

18. The composition of claim 17, wherein at least 2 branches are attached to the core.

19. The composition of claim 17, wherein at least 3 branches are attached to the core.

20. The composition of claim 17, wherein at least 4 branches are attached to the core.

21. The composition of claim 5, wherein the core has the structure 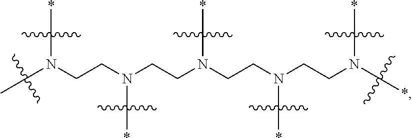
wherein * indicates a point of attachment of the core to a branch of the plurality of branches or H.

22. The composition of claim 21, wherein at least 4 branches are attached to the core.

23. The composition of claim 21, wherein at least 5 branches are attached to the core.

24. The composition of claim 21, wherein at least 6 branches are attached to the core.

25. The composition of claim 5, wherein A¹ is —O— or —NH—.

26. The composition of claim 25, wherein A¹ is —O—.

27. The composition of claim 5, wherein A² is —O— or —NH—.

28. The composition of claim 27, wherein A² is —O—.

29. The composition of claim 5, wherein Y³ is C₁-C₁₂ (e.g., C₁-C₆, such as C₁-C₃) alkylene.

30. The composition of claim 5, wherein the diacyl group independently at each occurrence comprises a structural formula 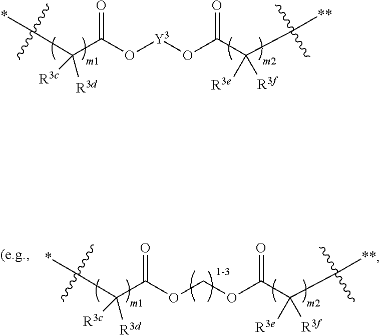
such as 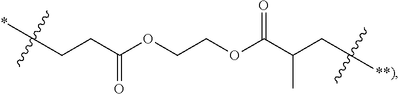
optionally wherein R^{3c}, R^{3d}, R^{3e}, and R^{3f} are each independently at each occurrence hydrogen or C₁-C₃ alkyl.

31. The composition of claim 5, wherein each terminating group is independently C₁-C₁₈ (e.g., C₄-C₁₈) alkenylthiol or C₁-C₁₈ (e.g., C₄-C₁₈) alkylthiol.

32. The composition of claim 5, wherein each terminating group is independently selected from those set forth in Table 3 or a subset thereof.

33. The composition of claim 1 or 2, wherein the ionizable cationic lipid is selected from those set forth in Table 4, or pharmaceutically acceptable salts thereof, or a subset of the lipids and the pharmaceutically acceptable salts thereof.

34. The composition of claim 1 or 2, wherein the ionizable cationic lipid is selected from those set forth in Table 4 or Table 5, or pharmaceutically acceptable salts thereof, or a subset of the lipids and the pharmaceutically acceptable salts thereof.

35. The composition of claim 1 or 2, wherein the lipid composition further comprises a phospholipid.

36. The composition of claim 35, wherein the phospholipid is at a molar percentage from about 8% to about 23%.

37. The composition of claim 1 or 2, wherein the lipid composition further comprises a steroid or steroid derivative.

38. The composition of claim 37, wherein the steroid or steroid derivative is at a molar percentage from about 15% to about 46%.

39. The composition of claim 1 or 2, wherein the ionizable cationic lipid is at a molar percentage from about 5% to about 30%.

40. The composition of claim 1 or 2, wherein the polymer-conjugated lipid is at a molar percentage from about 0.5% to about 10%.

41. The composition of claim 1 or 2, wherein the polymer-conjugated lipid is at a molar percentage from about 1% to about 10%.

42. The composition of claim 1 or 2, wherein the polymer-conjugated lipid is at a molar percentage from about 2% to about 10%.

43. The composition of claim 1 or 2, wherein the SORT lipid is at a molar percentage from about 20% to about 65%.

44. The composition of claim 1 or 2, wherein the therapeutic agent is a polynucleotide; and wherein a molar ratio of nitrogen in the lipid composition to phosphate in the polynucleotide (N/P ratio) is no more than about 20:1.

45. The composition of claim 44, wherein the N/P ratio is from about 5:1 to about 20:1.

46. The composition of claim 1 or 2, wherein a molar ratio of the therapeutic agent to total lipids of said lipid composition is no more than about 1:1, 1:10, 1:50, or 1:100.

47. The composition of claim 1 or 2, wherein at least about 85% of said therapeutic agent is encapsulated in particles of said lipid compositions.

48. The composition of claim 1 or 2, wherein said lipid composition comprises a plurality of particles characterized by one or more characteristics of the following:
(1) a (e.g., average) size of 100 nanometers (nm) or less;
(2) a polydispersity index (PDI) of no more than about 0.2; and
(3) a negative zeta potential of −10 millivolts (mV) to 10 mV.

49. The composition of claim 1 or 2, wherein said lipid composition has an apparent ionization constant (pKa) outside a range of 6 to 7.

50. The composition of claim 49, wherein said apparent pKa of said lipid composition is of about 7 or higher.

51. The composition of claim 49, wherein said apparent pKa of said lipid composition is of about 8 or higher.

52. The composition of claim 49, wherein said apparent pKa of said lipid composition is from about 8 to about 13.

53. A method for targeted delivery of a therapeutic agent to a spleen cell, the method comprising (e.g., systemically) administering a composition according to claim 1 or 2, thereby providing an effective amount or activity of said therapeutic agent in said spleen cell of said subject that is at least 1.1-fold greater than a corresponding amount or activity of said therapeutic agent achieved in a lung cell of said subject.

54. The method of claim 53, wherein the effective amount or activity of said therapeutic agent in said spleen cell of said subject that is at least 1.1-fold greater, at least 2.5-fold greater, at least 3.5-fold greater, at least 10-fold greater, at least 5.5-fold greater, at least 10-fold greater, at least 15-fold greater, at least 20-fold greater, or at least 50-fold greater than a corresponding amount or activity of said therapeutic agent achieved in a lung cell of said subject.

55. A method for targeted delivery of a therapeutic agent to a lung cell, the method comprising (e.g., systemically) administering a composition according to claim 1 or 2, thereby providing an effective amount or activity of said therapeutic agent in said lung cell of said subject that is at least 1.1-fold greater than a corresponding amount or activity of said therapeutic agent achieved in a spleen cell of said subject.

56. The method of claim 55, wherein said effective amount or activity of said therapeutic agent in said lung cell of said subject is at least 1.1-fold greater, at least 5-fold greater, at least 10-fold greater, at least 15-fold greater, at least 18-fold greater, at least 20-fold greater, or at least about 50-fold greater than a corresponding amount or activity of said therapeutic agent achieved in a liver cell of said subject.
